(19) United States
(12) Patent Application Publication
(10) Pub. No.: US 2024/0310364 A1
(43) Pub. Date: Sep. 19, 2024

(54) TUNABLE DYNAMIC AND NON-DYNAMIC HYDROGEL SYSTEMS

(71) Applicant: The Johns Hopkins University, Baltimore, MD (US)

(72) Inventors: Sharon GERECHT, Baltimore, MD (US); Rahel SCHNELLMANN, Baltimore, MD (US); Zhao WEI, Baltimore, MD (US); Eugenia VOLKOVA, Baltimore, MD (US)

(73) Assignee: The Johns Hopkins University, Baltimore, MD (US)

(21) Appl. No.: 18/571,126

(22) PCT Filed: Jul. 15, 2022
(86) PCT No.: PCT/US2022/073803

§ 371 Date: Dec. 15, 2023

Related U.S. Application Data
(60) Provisional application No. 63/222,195, filed on Jul. 15, 2021.

(51) Int. Cl. G01N 33/50; C12N 5/09

(52) U.S. Cl. CPC G01N 33/5082; C12N 5/0693; C12N 2503/04; C12N 2513/00; C12N 2533/54; C12N 2533/70; C12N 2537/10

(57) ABSTRACT

Disclosed are hydrogel systems with tunable stress-relaxation properties prepared from a polypeptide with one or more primary cross-linkable groups and a polysaccharide with one or more secondary cross-linkable groups. The resulting hydrogel can be tuned to have either dynamic or non-dynamic characteristics depending on the cross-linking groups employed.

### U.S. GOVERNMENT SUPPORT

This invention was made with Government support under grant number CA210173 from the National Institutes of Health/National Cancer Institute Center for Cancer Physics. The government has certain rights in this invention.

### BACKGROUND OF THE INVENTION

### Area of the Art

The present invention relates to hydrogel systems with tunable stress-relaxation properties. The hydrogels are prepared from a polypeptide with one or more primary cross-linkable groups and a polysaccharide with one or more secondary cross-linkable groups. The hydrogel is formed by reacting the primary cross-linkable group with the secondary cross-linkable group. The resulting hydrogel can be tuned to have either dynamic or non-dynamic characteristics depending on the cross-linking groups employed.

### Description of the Background Art

Advancements in tissue engineering, aimed at generating complex tissues in vitro, are progressing alongside the development of hydrogel materials that mimic aspects of three-dimensional (3D) physiological tissue microenvironments (Discher et al., 2005; Engler et al., 2004; Loebel et al., 2019; Madl et al., 2017; Tang et al., 2018). Vascularization is a critical step in understanding and controlling the assembly of functional healthy tissues (Li et al., 2017; Ronaldson-Bouchard and Vunjak-Novakovic, 2018; Zhang et al., 2016), as well as uncovering the mechanism governing cancer angiogenesis and metastasis via intravasation and extravasation processes (Boussommier-Calleja et al., 2019; Jeon et al., 2015; McCoy et al., 2019).

Cancer is the second leading cause of death in the world (Global Health Estimates, 2016). Growing tumors exert forces on their surroundings that can have consequences for cancer progression. Soft tissue sarcomas (STS) are a rare, heterogeneous group of malignant cancers of mesenchymal origin (Yoon et al., 2006; Singer et al., 2000). Approximately 13,000 new sarcoma cases are diagnosed in the United States annually; despite current treatment methods, 25%-50% of patients develop recurrent metastatic disease (Jemal et al., 2010; Wasif et al., 2011; Italiano et al., 2010). Most patients present with a localized tumor for which surgical resection is the main treatment (Demetri et al., 2010). Additionally, radiation therapy and chemotherapy are often given either pre- or post-operatively and prove essential when treating patients with locally-advanced or high-risk disease presentation (Karavasilis et al., 2008; Linch et al., 2014; Bramwell et al., 2003). Current clinical data suggests that undifferentiated pleomorphic sarcoma (UPS) is one of the most aggressive STS subtypes, which frequently result in lethal pulmonary metastases insensitive to radiation and chemotherapy (Hoang et al., 2018). Prior studies have confirmed that STS progression and metastasis are informed by microenvironmental cues, such as extracellular matrix (ECM) remodeling, stiffness modulation, cell-to-cell/matrix interaction, and signaling factors (Junttila et al., 2013; Quail et al., 2013; Fukumura et al., 2007). However, there is a need to better understand how the ECM, along with the aforementioned cues, governs cellular behavior.

The matrix's ability to relax from deformation under constant strain is called stress relaxation or matrix relaxation (Fung, 2013). Recently, researchers have made efforts to understand the role of time-dependent aspects of ECM mechanics on regulating cellular behaviors (Chaudhuri et al., 2016; Gong et al., 2018; Nam et al., 2019; Nam, Stowers, et al., 2019). Recent results demonstrate that stiffness and stress relaxation regulate cellular responses including cell motility, mesenchymal stem cell differentiation, and the cell's ECM sensing capability (Farley et al., 2015). It has been shown that 20-day old mUPS procollagen-lysine, 2-oxogluterate 5-dioxygenase 2 (PLOD2)-deficient tumors, which possess defects in collagen maturation, have slower relaxation times than their knockdown controls, Scr21. Rapid stress relaxation upregulates PLOD2 expression via TGFβ-SMAD2 signaling and forms a feedback loop between hypoxia and the matrix (Lewis et al., 2019).

Despite the recent advancements in the field, there is a need to expand the range relaxation times to address the impact of cancer-associated matrix changes. Stress relaxation times for biological tissues vary over an order of magnitude in their half-times (T_{1/2}), estimated to range from 10 to 200 seconds (Chaudhuri et al., 2016). Most conventional natural collagen hydrogel models offer a narrow range for T_{1/2}, while the model disclosed herein recapitulates highly dynamic stress relaxation environments where remodeling changes stress relaxation by orders of magnitude.

### SUMMARY OF THE INVENTION

In one aspect, the invention includes a hydrogel prepared from i) a polypeptide with one or more of a primary cross-linkable group; and ii) a polysaccharide with one or more of a secondary cross-linkable group; wherein said hydrogel is formed by reacting the primary cross-linkable group with the secondary cross-linkable group.

In certain embodiments, the polypeptide is selected from gelatin, soy protein, albumin, and collagen. In an exemplary embodiment, the polypeptide is gelatin.

In certain embodiments the polysaccharide is selected from hyaluronic acid, agarose, Alginate, cellulose and derivatives thereof, methylcellulose, chitosan, and dextran. In an exemplary embodiment, the polysaccharide is dextran.

In certain embodiments the primary cross-linkable group is an acylhydrazine and the secondary cross-linkable group is an aldehyde. In other embodiments, the first and secondary cross-linkable groups are (meth)acrylates. In certain embodiments, the secondary cross-linkable group is a glycidyl (meth)acrylate.

In an exemplary embodiment, the polypeptide is gelatin and the primary cross-linkable group is an acylhydrazine; and the polysaccharide is dextran and the secondary cross-linkable group is an aldehyde.

In another exemplary embodiment, the polypeptide is gelatin and the primary cross-linkable group is a methacrylate; and the polysaccharide is dextran and the secondary cross-linkable group is a glycidyl (meth)acrylate.

In one aspect, the hydrogel includes a cell or a therapeutic agent.

In one aspect, the hydrogel may contain up to 20 wt. % of the primary cross-linkable group, for example, up to 15 wt. %, up to 10 wt. %, or up to 5 wt. % of the primary cross-linkable group. In another aspect, the hydrogel may contain up to 5 wt. % of the secondary cross-linkable group, for example, up to 2.5 wt. %, up to 1 wt. %, or up to 0.5 wt. % of the secondary cross-linkable group. In one aspect, the hydrogel contains up to 20 wt. % of the primary cross-linkable group and up to 5 wt. % of the secondary cross-linkable group. In one aspect, the hydrogel contains up to 5 wt. % of the primary cross-linkable group and up to 0.5 wt. % of the secondary cross-linkable group. In an exemplary embodiment, the hydrogel contains 0.5 wt. % of the secondary cross-linkable group. In another exemplary embodiment, the hydrogel contains 0.25 wt. % of the secondary cross-linkable group.

In another aspect the invention includes a method of making the hydrogel by combining the gelatin, dextran and an optionally a photoinitiator in saline. The mixture can be made by dissolving gelatin in saline to form a first solution; dissolving dextran in saline to form a second solution; mixing together the first solution with the second solution to form a mixture; and optionally adding a photoinitiator. In other embodiments, the gelatin, dextran, and optional photoinitiator can be mixed together in a single saline solution. The mixture is then cross-linked, optionally with heating to form the hydrogel.

The crosslinking may be accomplished by thermal, chemical, or photochemical methods. In photochemical methods which may include a photoinitiator, the crosslinking may include exposing the mixture to light, for example UV light.

In another aspect the invention includes a method of growing vasculature or other tissue, healing wounds, delivering cells, or delivering a therapeutic agent by administering the hydrogel to a subject in need thereof.

In another aspect the invention includes a method of investigating tissue assembly and morphogenesis by measuring a parameter of one or more of vascularization, angiogenesis, cellular migration, stress relaxation, interaction of cellular peptides, and cellular responses to a stimulus in a first hydrogel and a second hydrogel, such that the first hydrogel is a dynamic hydrogel and the second hydrogel is a non-dynamic hydrogel; and the first hydrogel and the second hydrogel have similar stiffness; and comparing the parameter measured in the first hydrogel to the parameter measured in the second hydrogel; and determining a property of tissue assembly and morphogenesis by the comparison.

In another aspect, the invention is a method of investigating tumor growth, single-cell matrix interactions stress relaxation time changes in tumor-matrix interactions by measuring filopodia-like protrusions in a first hydrogel and a second hydrogel. The first hydrogel is a dynamic hydrogel and the second hydrogel is a non-dynamic hydrogel. The first hydrogel and the second hydrogel may have similar stiffness. The method includes comparing the parameter measured in the first hydrogel to the parameter measured in the second hydrogel; and determining a property of tissue assembly and morphogenesis by the comparison.

In any embodiment of the invention, the dynamic hydrogel can includes a i) a polypeptide with a primary cross-linkable group being an acylhydrazine; and ii) a polysaccharide with a secondary cross-linkable group being an aldehyde; such that the hydrogel is formed by reacting the primary cross-linkable group with the secondary cross-linkable group; and the non-dynamic hydrogel includes i) a polypeptide with a primary cross-linkable (meth)acrylate group; and ii) a polysaccharide with a secondary cross-linkable (meth)acrylate group; such that the hydrogel is formed by reacting the primary cross-linkable (meth)acrylate group with the secondary cross-linkable (meth)acrylate group.

In one aspect of the method, the weight ratio of polypeptide to polysaccharide in the dynamic hydrogel and the weight ratio of polypeptide to polysaccharide in the non-dynamic hydrogel are about the same. In another aspect of the method, the weight ratio of polypeptide to polysaccharide in the dynamic hydrogel and the weight ratio of polypeptide to polysaccharide in the non-dynamic hydrogel can be the same or different and can each up to about 20:1, for example about 15:1, about 10:1 or about 5:1. In another aspect of the method, the weight ratio of polypeptide to polysaccharide in the dynamic hydrogel and the weight ratio of polypeptide to polysaccharide in the non-dynamic hydrogel are the same and can be about 20:1, for example about 15:1, about 10:1 or about 5:1. In another aspect of the method the weight ratio of polypeptide to polysaccharide in the dynamic hydrogel and the weight ratio of polypeptide to polysaccharide in the non-dynamic hydrogel are each about a 10:1. In another aspect of the method, the weight ratio of polypeptide to polysaccharide in the dynamic hydrogel and the weight ratio of polypeptide to polysaccharide in the non-dynamic hydrogel are each about a 20:1. In an exemplary embodiment of the method, the polypeptide is gelatin, and the polysaccharide is dextran.

In another aspect the invention includes a method of promoting angiogenesis, tissue regeneration, reperfusion or perfusion in a subject in need thereof by administering the hydrogel. In another embodiment of the method, the hydrogel contains a cell or a therapeutic agent.

In another aspect, the invention is a method of promoting spheroid growth, single-cell matrix interactions, focal adhesions, or filopodia-like protrusions by administering a hydrogel of the invention as herein described.

### DESCRIPTION OF THE FIGURES

FIG. 1 shows the design and characterization of dynamic network hydrogels (D-Hydrogels, also referred to herein as quick relaxing hydrogels) and non-dynamic network hydrogels (N-Hydrogels, also referred to herein as slow relaxing hydrogels). Significance levels were set at n.s. p>0.05, *p % 0.05, and ** p % 0.01.

(A) is a schematic depicting networks of D-hydrogel (i) crosslinked by the covalent bonds of imine and acylhydrazine bonds between Gtn-ADH and Dex-CHO, and N-hydrogel (ii) crosslinked by the covalent bonds between Gtn-MA and Dex-GMA.
(B) shows a storage moduli (G′) of D-hydrogels and N-hydrogels with different stiffness (D-hydrogels are in red and N-hydrogels are in blue).
(C) shows stress relaxation curves of D-hydrogels and N-hydrogels with different stiffness. Stress is normalized to the initial stress.
(D) shows the quantification of the timescale at which the stress is relaxed to half its original value, t_{1/2}. from stress relaxation tests of D-hydrogels and N-hydrogels with differing stiffness.
(E) shows no significant change in stiffness, G′, of D-hydrogels and N-hydrogel along 3 days of incubation in endothelial growth medium-2 (EGM-2).
(F) shows a significant increase in stress relaxation time over 3 days in culture in EGM-2.
(G) shows half-stress relaxation time, T_{1/2}, is maintained lower than in N-hydrogel along the 3-day culture period.
(H) shows the ¹H NMR spectrum (D₂O) of Gelatin (Gtn) modified with adipic acid dihydrazide (Gtn-ADH).
(I) shows the ¹H NMR spectrum (D₂O) of Gelatin (Gtn) modified with methacrylate (Gtn-MA).
(J) shows the ¹H NMR spectrum (D₂O) of Dextran (Dex) modified with glycidyl methacrylate (Dex-MA).
(K) depicts rheological time sweep tests of the formation of hydrogels cross-linked by Gtn-ADH (5 wt %) or Gtn (5 wt %) with 0.5 w % Odex in 37° C. at a fixed frequency of 1 Hz and fixed strain of 0.1%.
(L) shows the formation of D-hydrogels cross-linked by Gtn-ADH (5 wt %) or Gtn (5 wt %) with 0.25 w % Odex in 37° C. at a fixed frequency of 1 Hz and fixed strain of 0.1%.
(M) shows the gelling time of hydrogels cross-linked by Gtn-ADH (5 wt %) or Gtn (5 wt %) with different concentrations of Odex. Significance levels were set at ****p≤0.0001.
(N) shows stress relaxation behaviors for D-hydrogels could be closely fitted to that of an empirical stretched exponential function, which closely agrees with the raw data (black).
(O) shows stress relaxation behaviors for N-hydrogels could be closely fitted to that of an empirical stretched exponential function, which closely agrees with the raw data (black).

FIG. 2 shows hydrogels with dynamic (D) and non-dynamic (N) networks differently modulate morphogenesis of encapsulated endothelial colony-forming cells (ECFCs). Significance levels were set at n.s. p>0.05, *p % 0.05, **p % 0.01, and ****p % 0.0001.

(A) shows a schematic depicting the vasculogenesis of ECFCs encapsulated in hydrogels from the initial step of vacuole or lumen formation, then to sprouting and branching, to the final tubulogenesis of the complex vascular bed.
(B) depicts light micrographic images showing phenotype changes of encapsulated ECFCs in D-hydrogels and N-hydrogels across 3 days in culture in EGM-2 media. Scale bars, 100 mm (larger images) and 20 mm (insets).
(C) shows the stiffness, G′, of D-hydrogels and N-hydrogels encapsulated with ECFCs along the 3-day culture period (D-hydrogels in red and N-hydrogels in blue).
(D) shows the T_{1/2} of D-hydrogels and N-hydrogels encapsulated with ECFCs along the 3-day culture period (D-hydrogels in red and N-hydrogels in blue).
(E) shows confocal maximum intensity projection images of vascular phenotypes in D-hydrogels and N-hydrogels after 3 days in culture (GFP-ECFCs in green, nuclei in blue) showing an extensive vascular bed formed in D-hydrogels. Scale bars, 100 mm.
(F) depicts quantitative analysis of vascular tube formation after 3 days in culture and shows higher mean and total tube length of ECFCs encapsulated in D-hydrogels compared with N-hydrogels (analysis using Imaris Filament Tracer; n=3 biological replicates with 5-6 images per replicate).
(G) depicts quantitative analysis of vascular tube formation after 3 days in culture and shows higher mean and total tube volume of ECFCs encapsulated in D-hydrogels compared with N-hydrogels (analysis using Imaris Filament Tracer; n=3 biological replicates with 5-6 images per replicate).
(H) depicts representative confocal maximum intensity projection with orthogonal views (top and right side) of luminal structures (indicated with asterisks) in D-hydrogels after 3 days in culture (F-actin in purple and nuclei in blue). Scale bars, 50 mm.
(I) shows magnified confocal maximum intensity projection images of vessels with orthogonal views (top and right side of the 2 images at left) of luminal structures (indicated with asterisks) in D-hydrogels after 3 days in culture (F-actin in purple and nuclei in blue). Scale bars, 50 mm.

FIG. 3 shows hydrogels with dynamic networks promote focal adhesion (FA) formation in encapsulated ECFCs. Significance levels were set at ***p % 0.001 and ****p % 0.0001.

(A) depicts representative immunofluorescence (IF) images of ECFCs (in green) with fluorescent beads (in purple) in D-hydrogels and N-hydrogels, and quantification of displacement and speed of the beads in time-lapse (n=15 cells from biological triplicates) (D-hydrogels in red and N-hydrogels in blue). Scale bars, 20 mm.
(B) shows maximum intensity projections of confocal images of cells 12 h after embedding into N- and D-hydrogels show protrusion formation and localization of pMLCs to actin in D-hydrogels. Cells embedded in N-hydrogels have a round shape without any protrusions and pMLCs localized to the nucleus (phalloidin in green, pMLC in red, DAPI in blue). Scale bars, 20 mm.
(C) depicts corresponding quantification (n=30 cells from biological triplicates) of normalized intensities (lower graph) and percentage of nuclear protein to overall protein levels (upper graph).
(D) depicts quantification of normalized intensities (lower graph) and percentage of nuclear protein to overall protein levels (upper graph) of cells embedded into D-and N-hydrogels after 24 h in culture.
(E) shows IF images of integrin 31 staining of the normalized intensities and FA areas showing more FA in ECFCs encapsulated within D-hydrogels compared to N-hydrogels, after day 1 in culture. Scale bars, 20 and 10 mm for higher magnification of FA.
(F) depicts the corresponding quantifications (F; n=30 cells from biological triplicates) of the normalized intensities (left graph) and FA areas (calculated as a percentage of the total cell area; right graph) showing more FA in ECFCs encapsulated within D-hydrogels compared to N-hydrogels, after day 1 in culture. Scale bars, 20 and 10 mm for higher magnification of FA.
(G) depicts real-time RT-PCR analysis showing higher integrin β1 and integrin V mRNA expression in ECFCs encapsulated in D-hydrogels compared with N-hydrogels after day 1 in culture.
(H) shows IF images of GFP-ECFCs encapsulated in D-hydrogels and N-hydrogels stained for vinculin after day 1 in culture. Scale bar, 20 and 10 mm for higher magnification of FA.
(I) depicts quantifications (n=30 cells from biological triplicates) of FA size, number, and areas using vinculin staining showing more FA in ECFCs encapsulated in D-hydrogels compared with N-hydrogels after day 1 in culture.
(J) depicts quantifications (n=60 cells from biological triplicates) of aspect ratio using F-actin staining, demonstrating ECFCs spreading to a higher degree when encapsulated in D-hydrogels compared with N-hydrogels after day 1 in culture.
FIG. 4 shows cell contraction mediates integrin clustering and subsequent vessel Formation inhibition of cell contractility with blebbistatin leads to a reduction in integrin cluster size. Significance levels were set at ***p % 0.001 and ****p % 0.0001.
(A) shows maximum intensity projections of confocal images showing reduced integrin cluster size and area coverage in cells treated with 60 mm blebbistatin after 24 h in culture (integrin β1 in red, nuclei in blue). Scale bars, 10 mm.
(B) depicts analysis of integrin cluster size, number per cell, area covered and relative intensity (n=30 cells from biological triplicates) of the normalized intensities (top left graph) and integrin area (calculated as a percentage of the total cell area; bottom right graph) showing smaller integrin clusters in ECFCs encapsulated within D-hydrogels treated with blebbistatin for 24 h.
(C) shows maximum intensity projections of representative confocal image of reduced lamellipodial extension in ECFCs in D-hydrogels treated with blebbistatin for 24 h (nuclei in blue, phalloidin in magenta). Scale bars, 10 mm.
(D) shows confocal projection images of day 3 (GFP-ECFC in green, nuclei in blue, and phalloidin in red), showing inhibition of vasculature formation in blebbistatin-treated cells compared to untreated controls. Scale bars, 100 mm. Quantitative analysis of vascular tube formation, in D-hydrogels and D-hydrogels treated with blebbistatin after 3 days shows a decrease in mean tube length, as well as mean and tube volume (analysis using Imaris Filament Tracer; n=3 biological replicates with 4 images per replicate).

FIG. 5 shows dynamic networks lead to the activation of FAK, matrix degradation via increased MMP expression and ECM deposition. Significance levels were set at *p % 0.05, **p % 0.01, ***p % 0.001, and ****p % 0.0001.

(A) shows representative IF images and quantifications of the normalized intensities of pFAK showing increased activation in ECFCs encapsulated in D-hydrogels compared to N-hydrogels (pFAK in red, nuclei in blue) (n=30 cells from biological triplicates). Scale bars, 20 mm.
(B) shows representative IF images of MT1-MMP stains showing higher expression of MT1-MMP in ECFCs encapsulated in D-hydrogels compared to N-hydrogels after 24 h in culture (MT1-MMP in red, nuclei in blue). Scale bars, 50 mm.
(C) depicts real-time RT-PCR analysis showing that ECFCs encapsulated in D-hydrogels highly express MT1-MMP, MMP-1, and MMP-9 mRNA compared to N-hydrogels (D-hydrogels in red, N-hydrogels in blue).
(D) shows light micrographic images of ECFCs encapsulated in D-hydrogels treated with MMP inhibitor GM6001, at a concentration of 0.1 mM after days 1 and 3 of culture, showing inhibition of sprouting and vasculature formation compared to untreated controls. Scale bars, 100 mm.
(E) shows the G⁰ of ECFC-loaded D-hydrogel controls and D-hydrogels treated with GM6001 along with a 3-day culture period.
(F) depicts real-time RT-PCR analysis showing that ECFCs encapsulated in D-hydrogels highly express collagen IV (ColIV) and laminin on day 3 of culture compared to N-hydrogels.
(G) shows representative confocal maximum intensity projection with orthogonal views (bottom and right side) of ColIV stains (in white/red, cells in green, nuclei in blue) after 3 days in D-hydrogels showing strong localization of ColIV at the basement membrane of the lumenized vessels. Lumens are indicated with an asterisk. Scale bars, 20 mm. For graphs: D-hydrogels are in red, D-hydrogels treated with GM6001 or cells before encapsulation are in gray.

FIG. 6 shows FAK activation in ECFCs is required for D-hydrogel remodeling and vascular morphogenesis.

(A) depicts light micrographic images of ECFCs encapsulated within D-hydrogels treated with FAK inhibitor 14 (FI 14) at a concentration of 10 mM after days 1 and 3 of culture and corresponding confocal projection images of day 3 (GFP-ECFC in green, nuclei in blue), showing inhibition of sprouting and vessel formation compared to untreated controls. Scale bars, 100 and 20 mm for the inset.
(B) depicts quantifications of ECFC aspect ratio showing cell spreading is reduced when encapsulated in D-hydrogels treated with FI 14 compared with control D-hydrogels, after 24 h in culture (n=60 cells from biological triplicates) (D-hydrogels in red, D-hydrogels treated with FI 14 in brown).
(C) depicts quantitative analysis of vascular tube formation in D-hydrogels and D-hydrogels treated with FI 14 after 3 days showing a decrease in mean and total tube length (analysis using Imaris Filament Tracer; n=3 biological replicates with 5-6 images per replicate).
(D) depicts quantitative analysis of mean and total tube volume in D-hydrogels and D-hydrogels treated with FI 14 after 3 days (analysis using Imaris Filament Tracer; n=3 biological replicates with 5-6 images per replicate).
(E) depicts RT-PCR analysis showing downregulation of integrin β1 and integrin aV mRNA expression in ECFCs in D-hydrogels treated with FI 14 compared to D-hydrogel controls after 24 h in culture. Significance levels were set at **p % 0.01, ***p % 0.001, and ****p % 0.0001.
(F) depicts quantification of normalized intensities (right) and percentage of nuclear protein to overall protein levels (left) of cells embedded into D-hydrogels and treated with 10 mm FI.
(G) depicts representative IF images showing lower expression for MT1-MMP in ECFCs encapsulated in D-hydrogels treated with FI 14 compared to control D-hydrogels after 24 h in culture (GFP in green, MT1-MMP in red, phalloidin in purple, and nuclei in blue). Scale bars, 20 mm.
(H) depicts RT-PCR analysis showing downregulation of MT1-MMP, MMP-1, and MMP-9 mRNA expression in ECFCs encapsulated in D-hydrogels treated with FI 14 compared to D-hydrogel controls after 24 h in culture.

FIG. 7 shows dynamic networks accelerate vasculogenesis in vivo and proposed molecular pathway of ECFCs in response to dynamic networks

(A) shows GFP-ECFC-loaded D-and N-hydrogels directly implanted subcutaneously in nude mice and retrieved after (i) day 3, (ii) day 5, and (iii) day 7 (n=3). Representative confocal images show GFP-ECFC (green) of the corresponding extracted hydrogels.
(B) shows FAK activation is increased in ECFC-loaded D-hydrogels compared with N-hydrogels in vivo on day 5, indicated by pFAK signal intensity (pFAK in red, nuclei in blue). Scale bars, 20 mm (i) and 5 mm (ii).
(C) shows Integrin cluster size is larger in ECFC-loaded D-hydrogels (₁₃1-integrin in green, some indicated by arrows, nuclei in blue) in vivo on day 5. Scale bars, 20 mm (i) and 5 mm (ii).
(D) depicts quantification of pFAK signal intensity (i) and average 1-integrin cluster size (ii).
(E) depicts representative histological images of CD31+ vessels infiltrating into acellular hydrogels in D-hydrogels compared to individual cells invading into the edge of N-hydrogels (indicated by arrows). Scale bars, 100 mm.
(F) shows vessels, labeled with lectin, infiltrating into D-hydrogels were perfused (indicated by arrows) with Evans blue dye injected intravenously. Scale bars, 100 mm. Significance levels were set at **p % 0.01, ***p % 0.001, and ****p % 0.0001.

(G) shows hydrogels with dynamic networks enable the rapid formation of FA in a stiffness-independent manner. Contrarily, static covalent hydrogels do not facilitate the formation of FA, leading to an abrogation of vascular morphogenesis. In both systems, ECFCs interact with the hydrogel-binding sites, leading to vacuole and lumen formation. (i) The rigidity of the non-dynamic matrix prevents the formation of integrin clusters via cell contractility inhibition. (ii) In the dynamic matrix, integrin β1 interaction with RGD-binding sites of the Gtn leads to the recruitment of FAK and other FA proteins. In a second step, pMLC-mediated actin contractility leads to the formation of larger integrin clusters. Integrin clustering and the recruitment of vinculin to the FAs leads to the formation of larger, stable FAs. These FAs allow for robust downstream signaling and further FAK activation. Activated FAK further contributes to cell contraction and integrin expression, promoting the formation of larger integrin clusters and taking part in robust downstream signaling. The activation of FAK leads to the upregulation of MT1-MMP, MMP-1, and MMP-9, resulting in matrix degradation and remodeling, allowing for the progression of ECFC vasculogenesis.

FIG. 8 shows the vascular morphogenesis in stiff hydrogels, diffusion rates and cytocompatibility in D- and N-hydrogels. Significance levels were set at n.s. p>0.05.

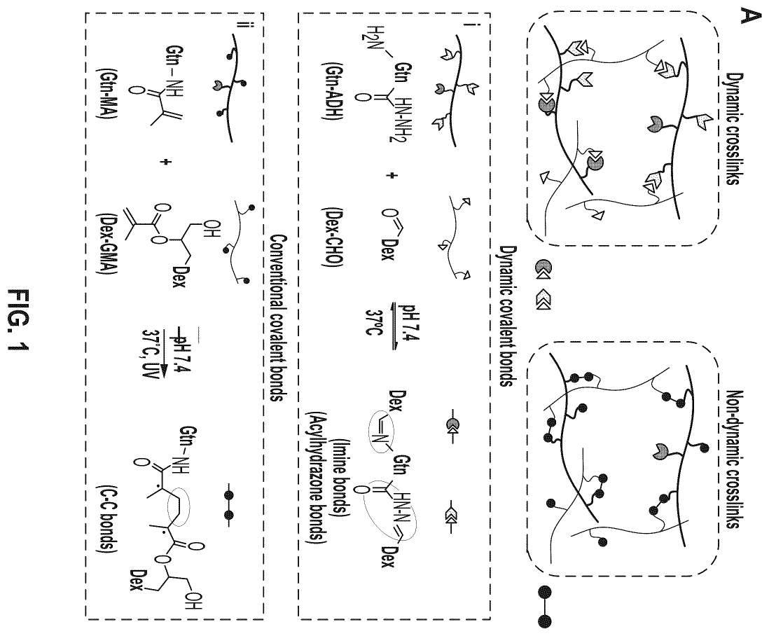

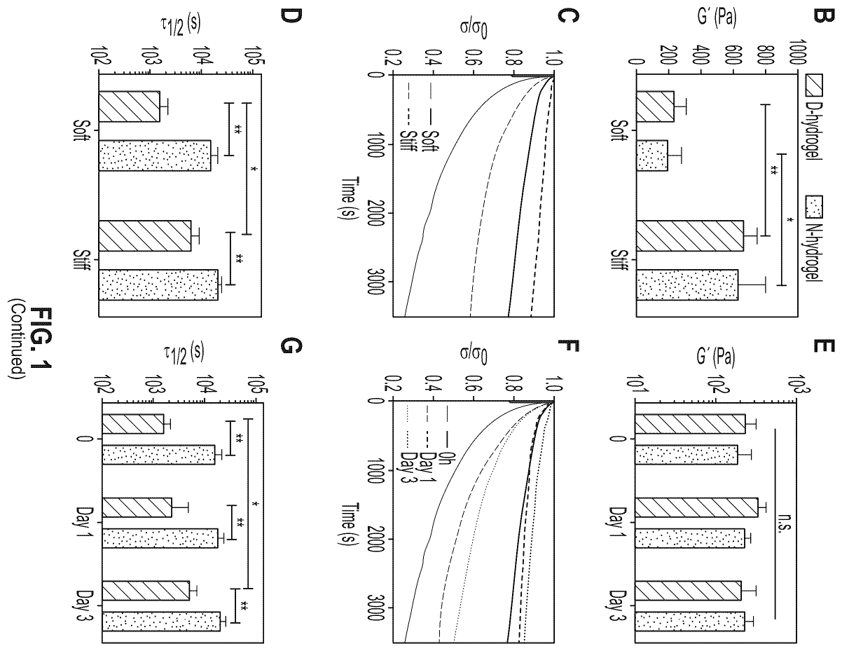

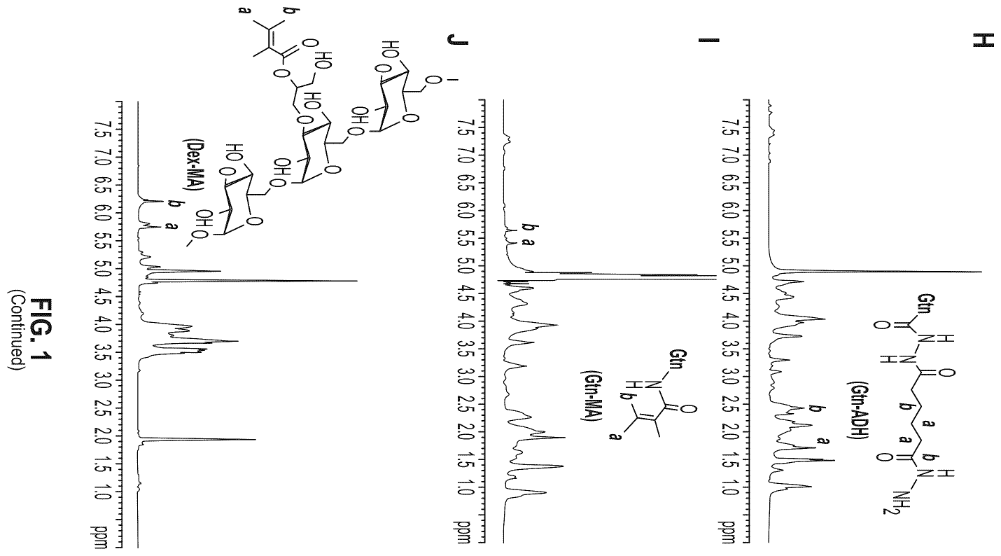

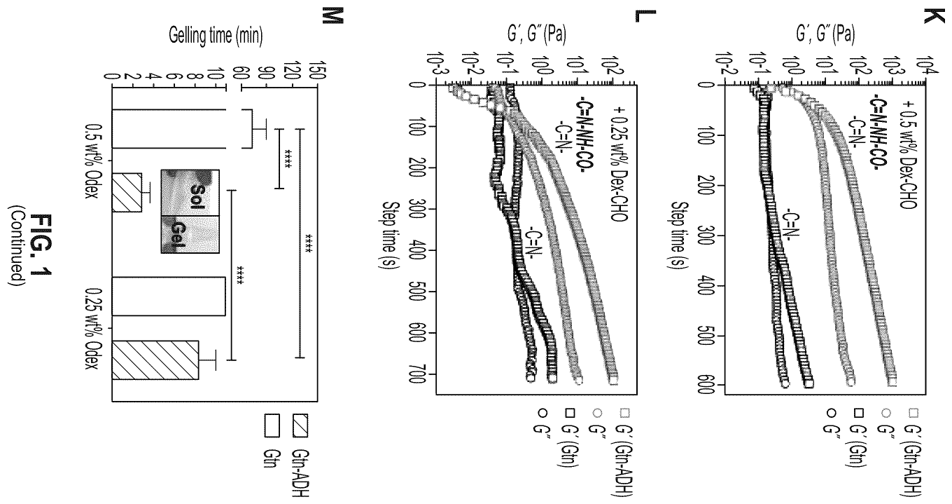

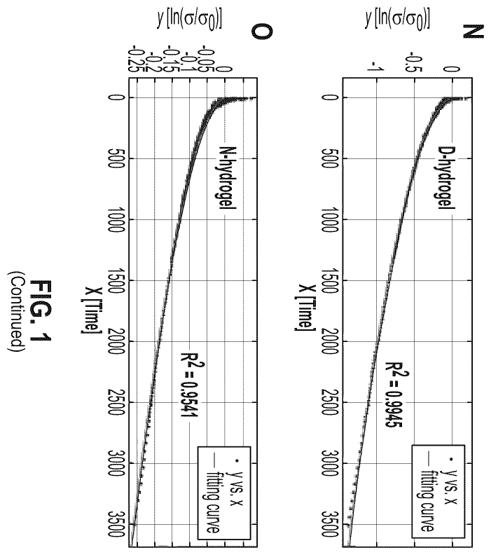

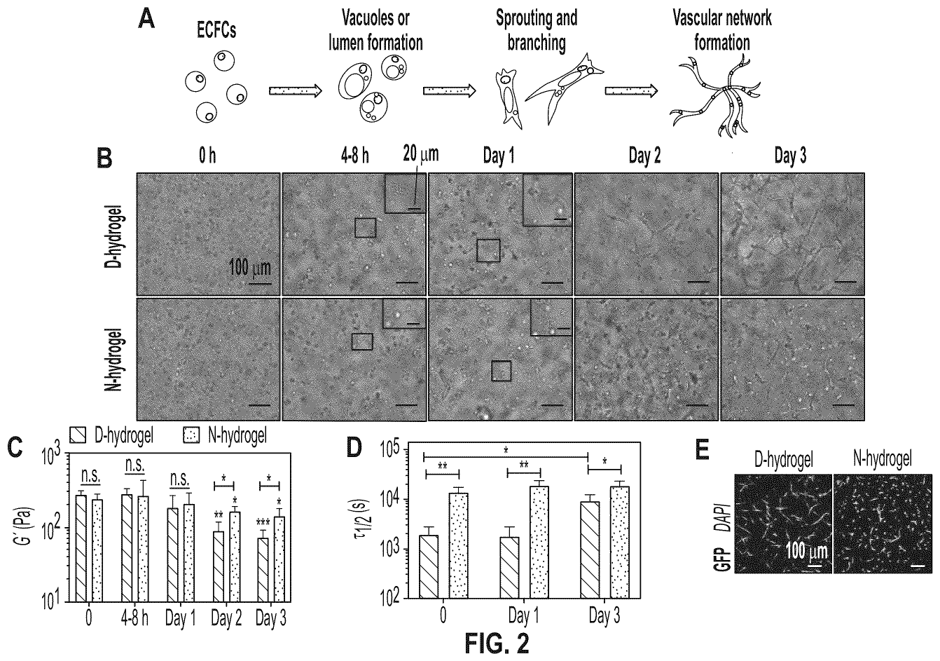

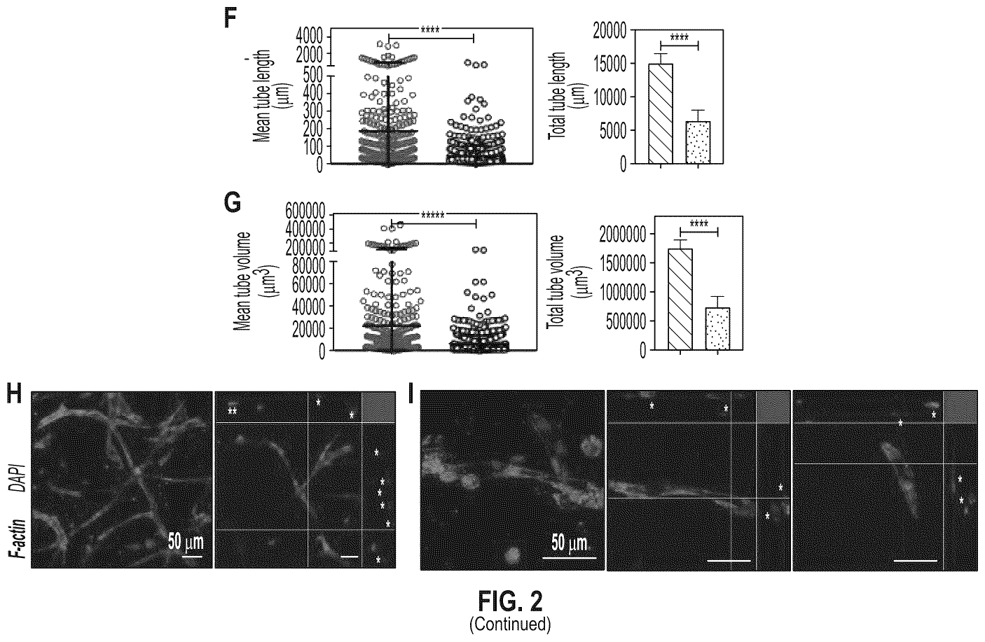

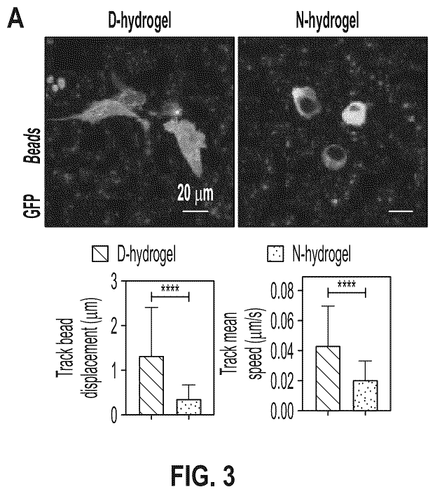

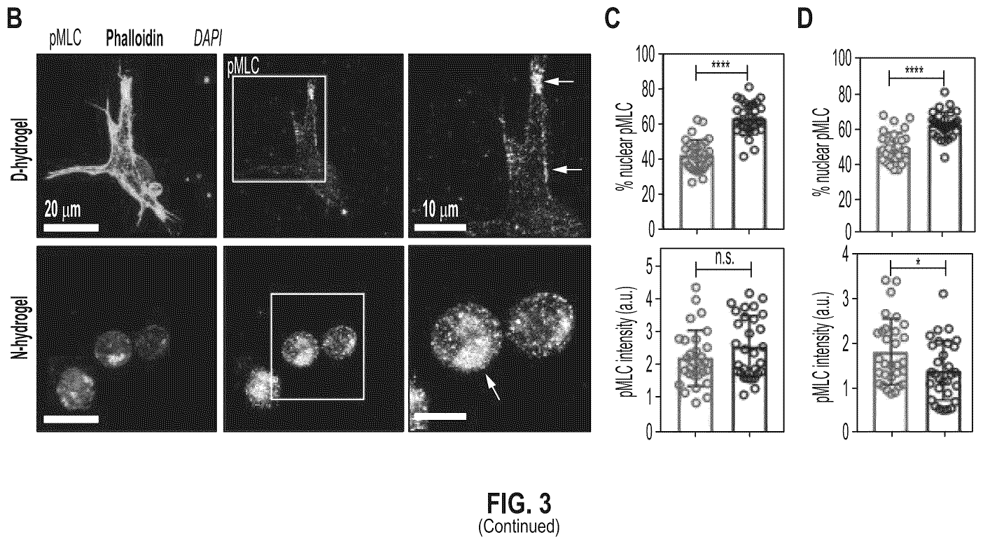

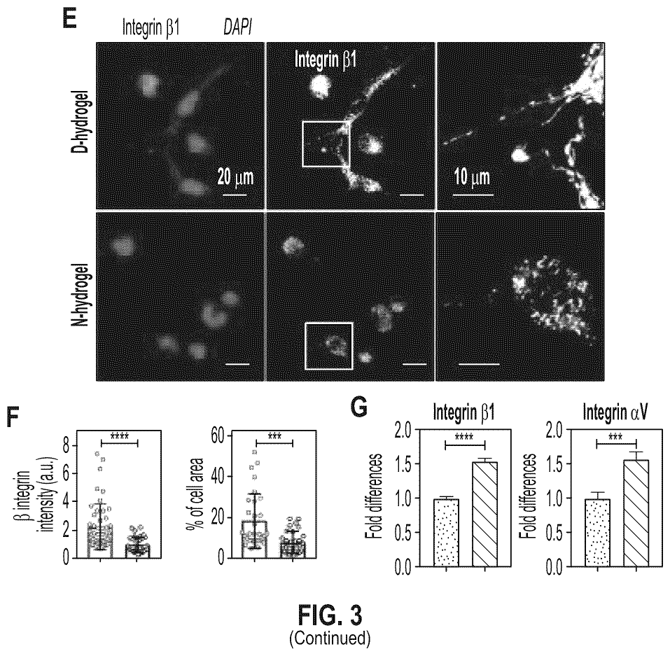

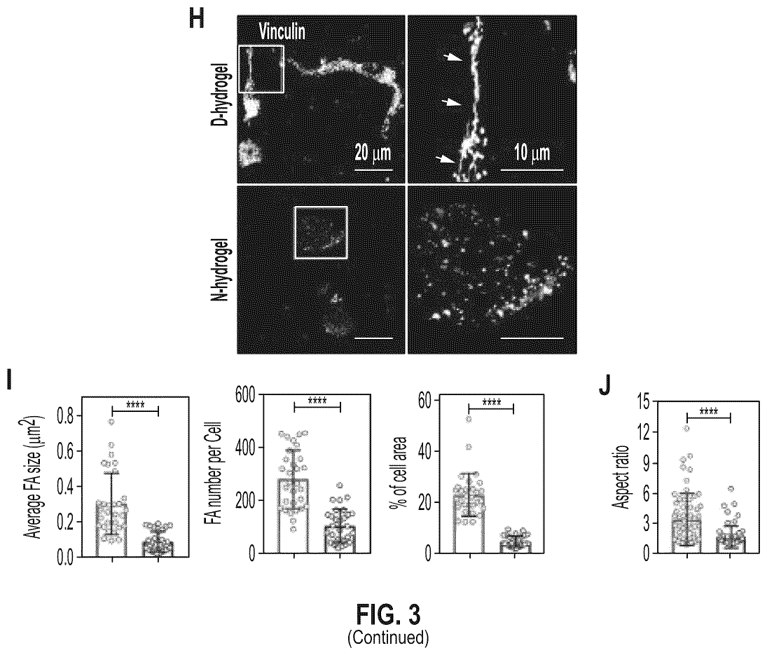

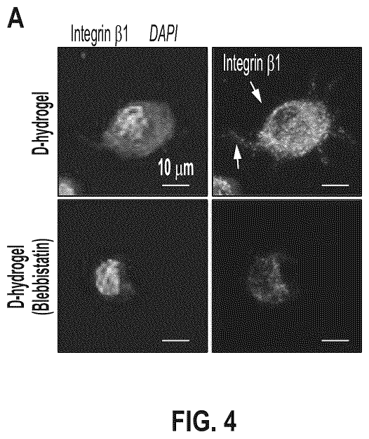

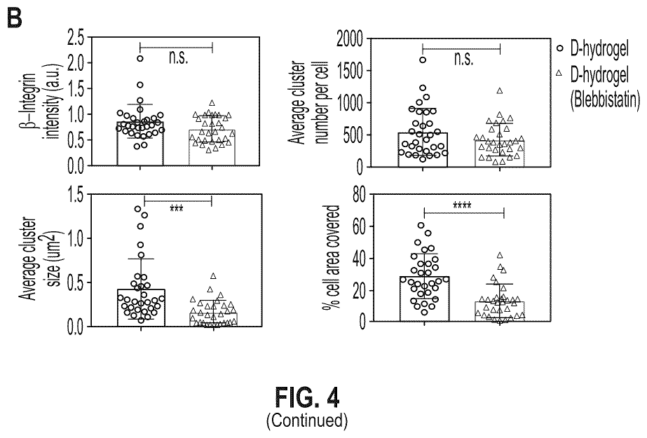

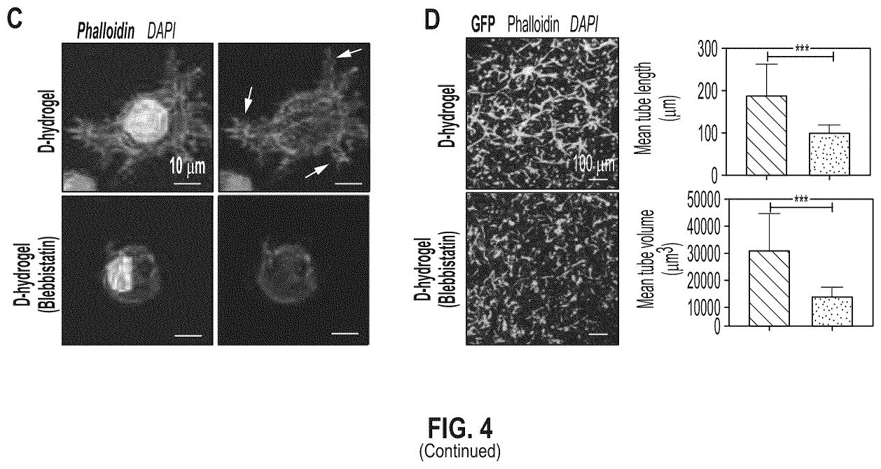

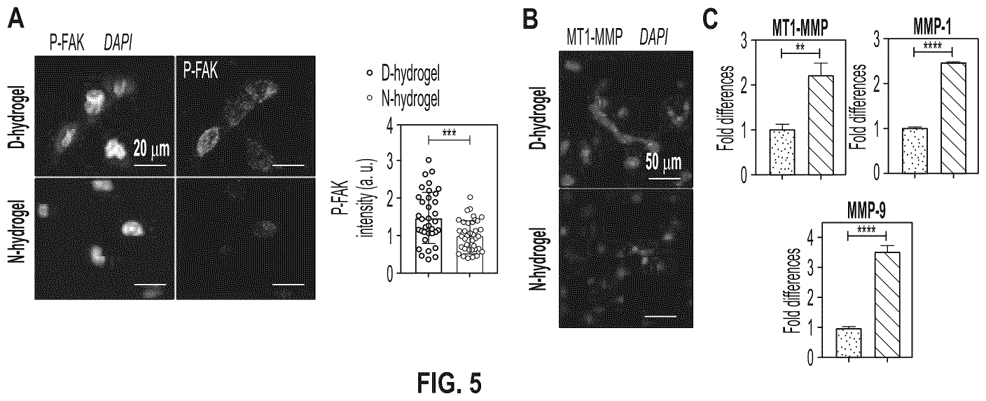

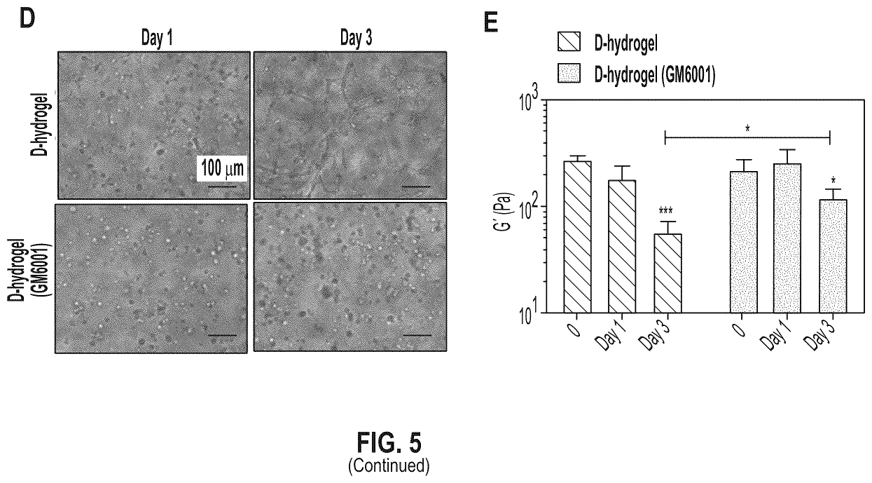

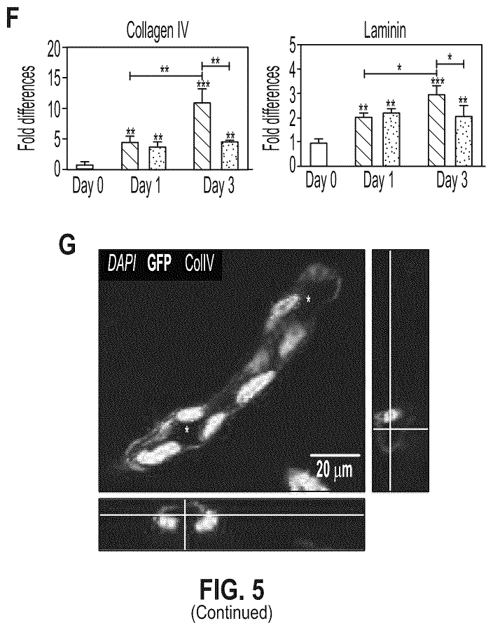

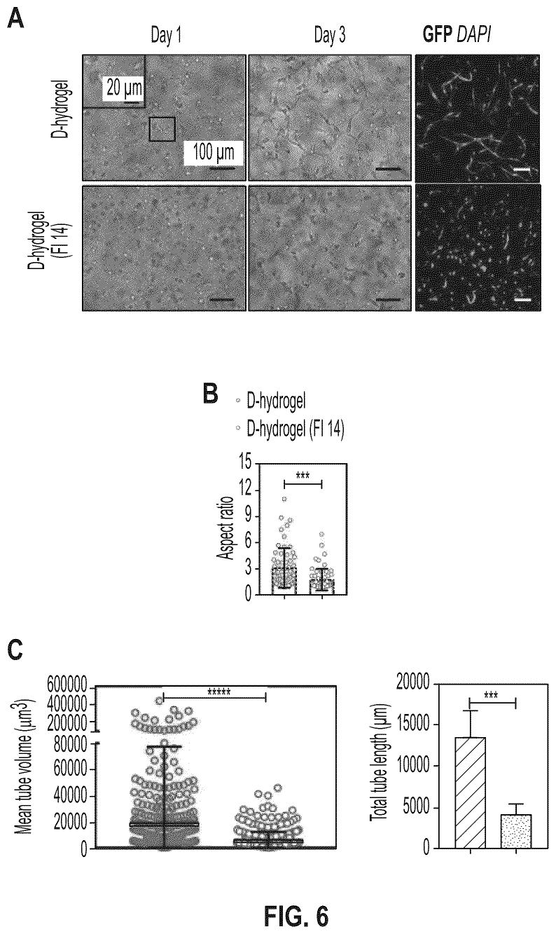

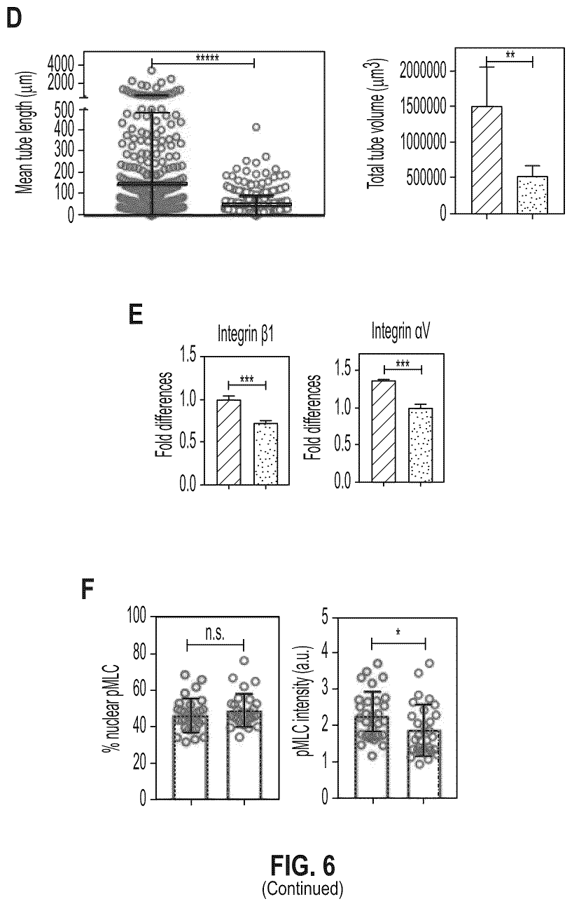

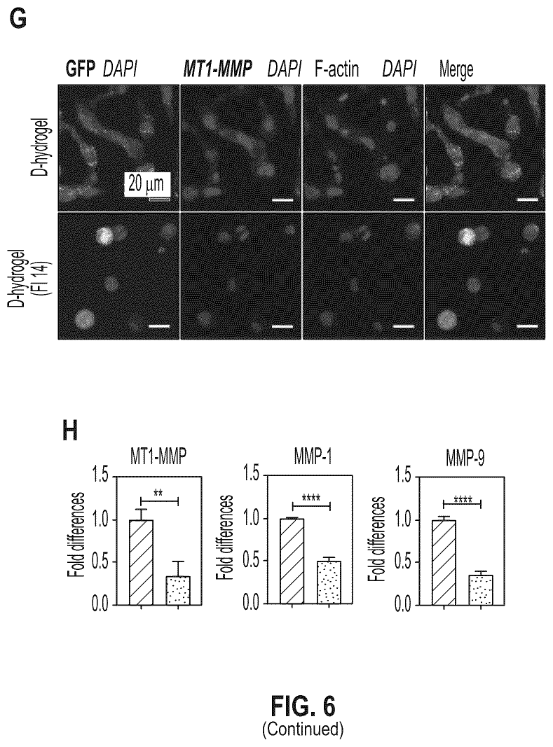

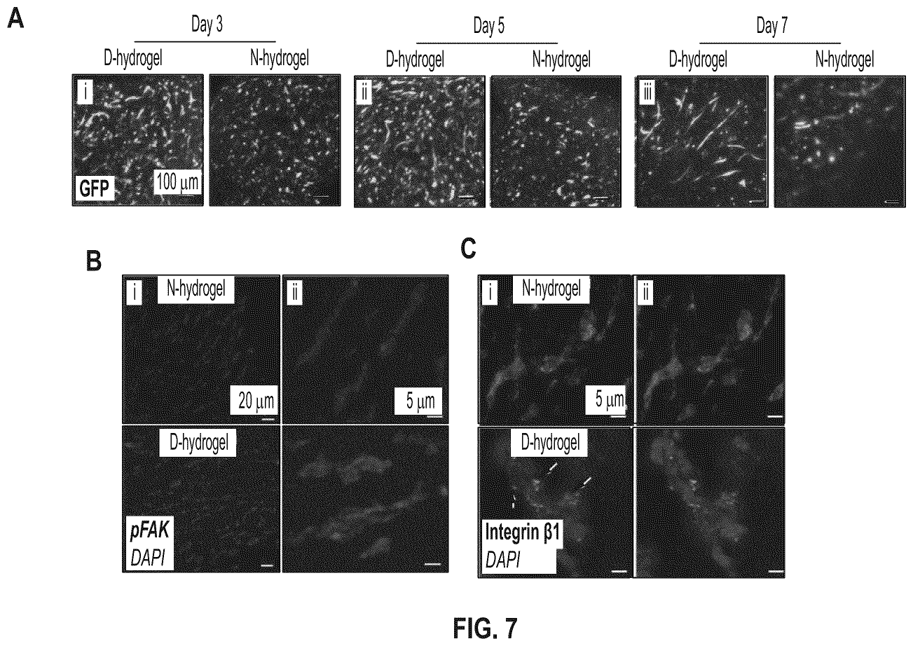

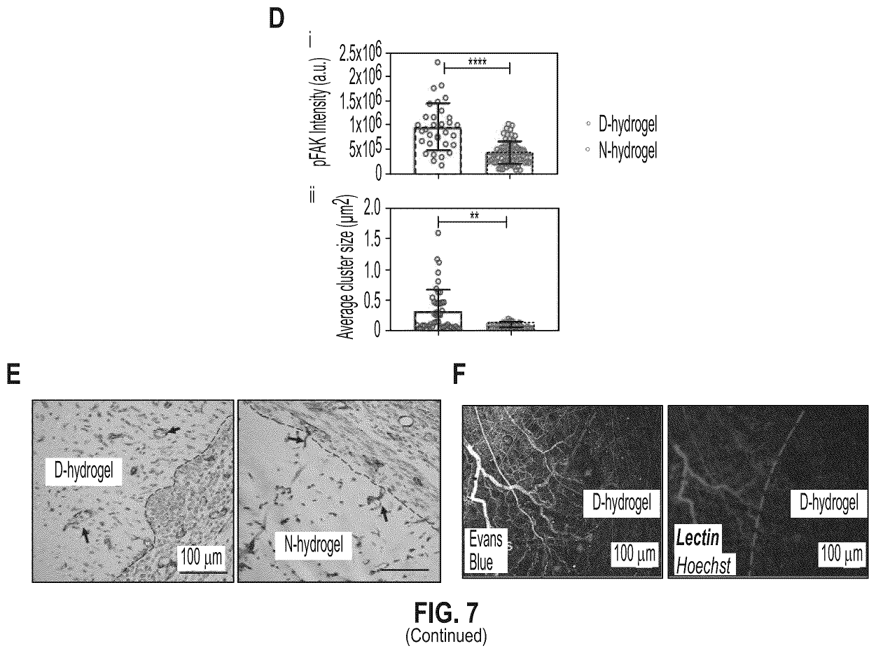

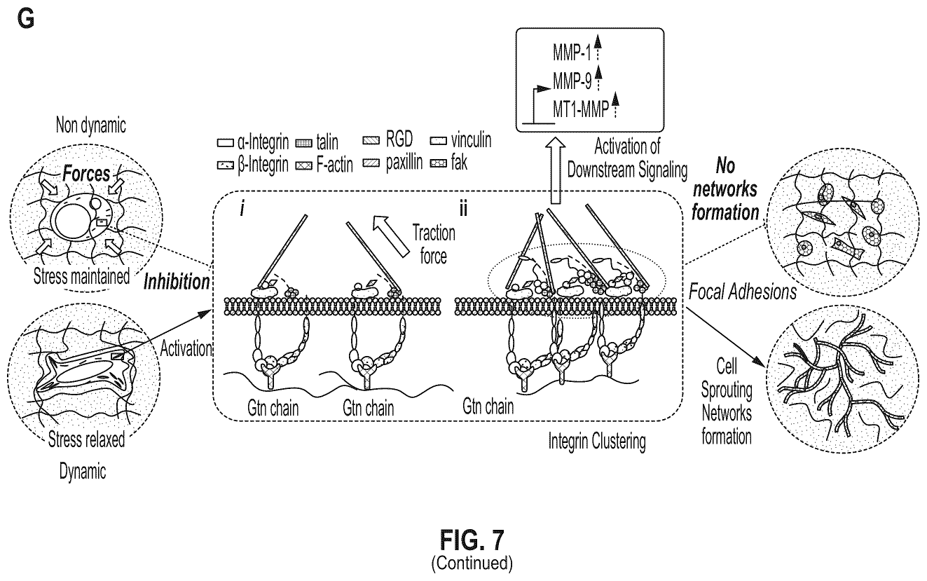

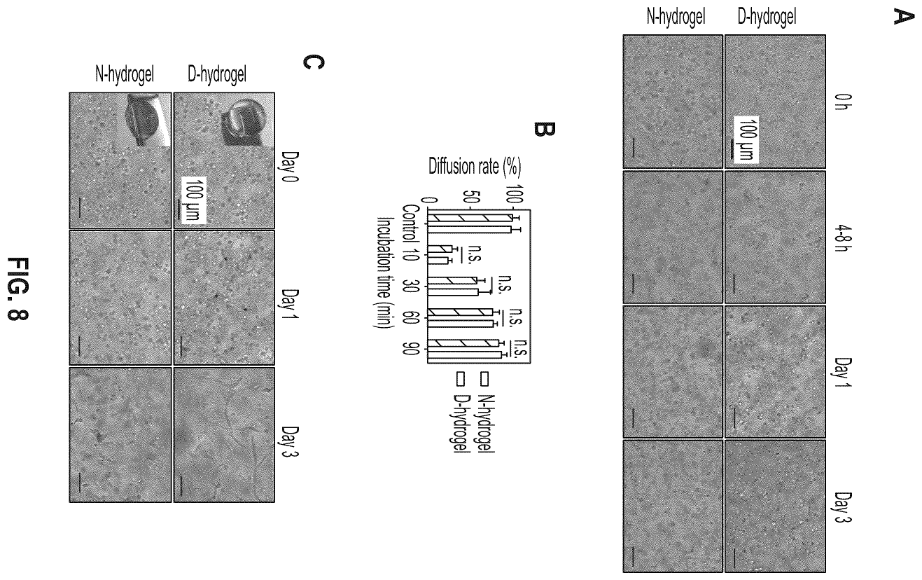

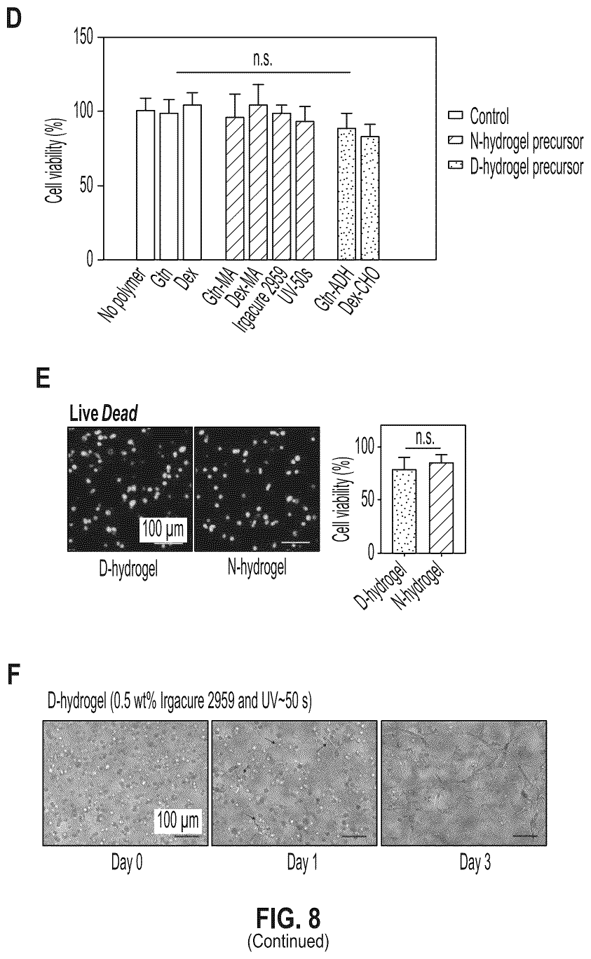

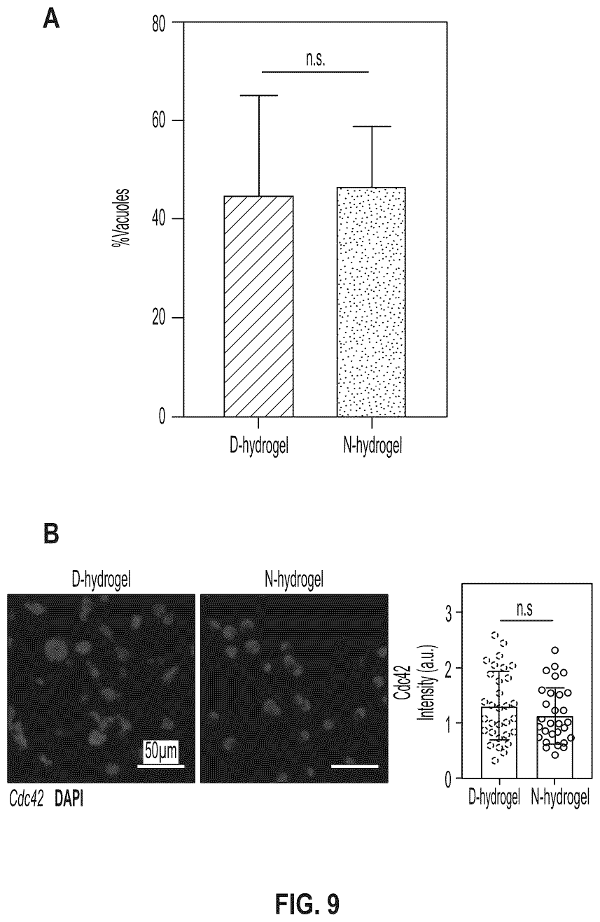

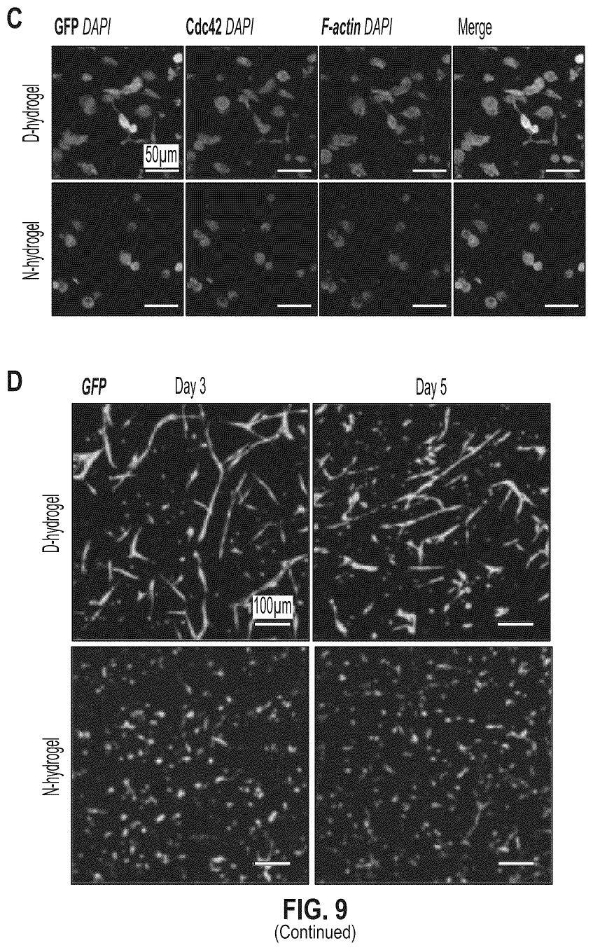

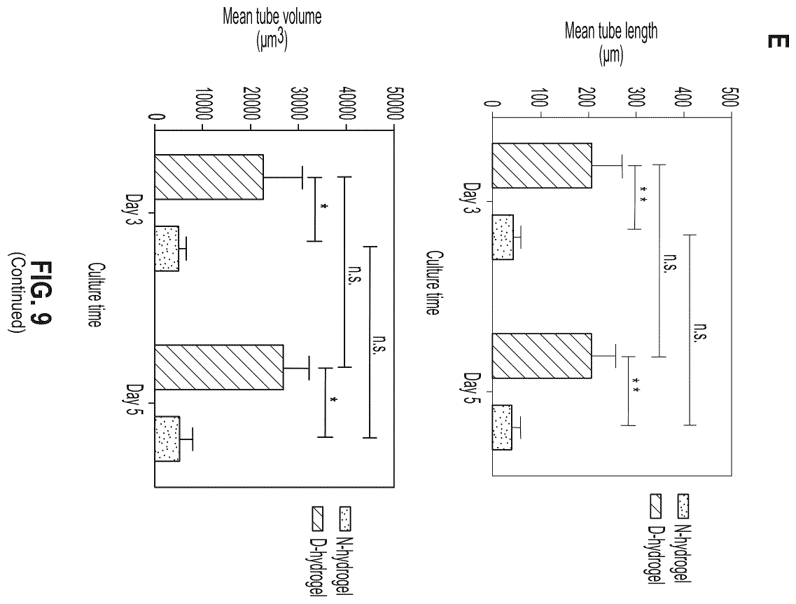

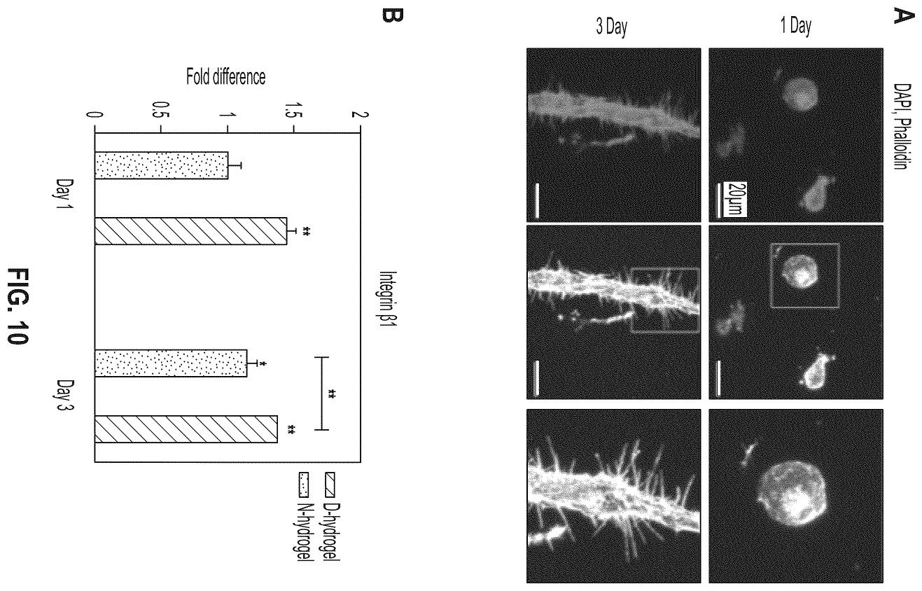

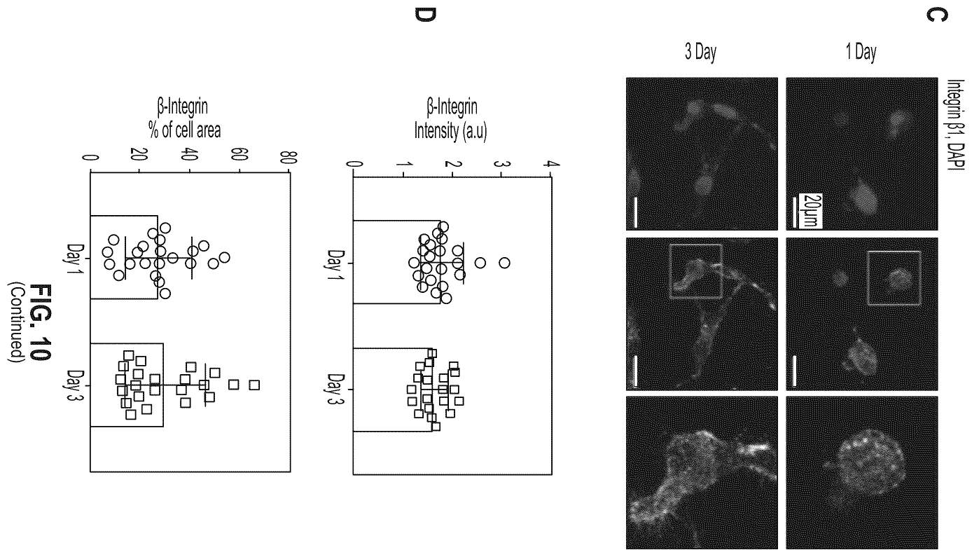

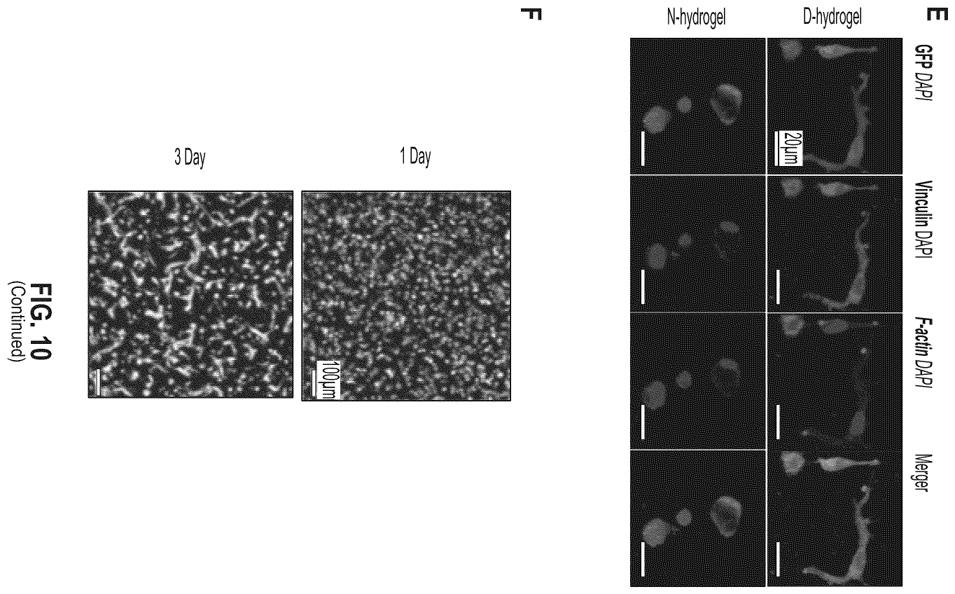

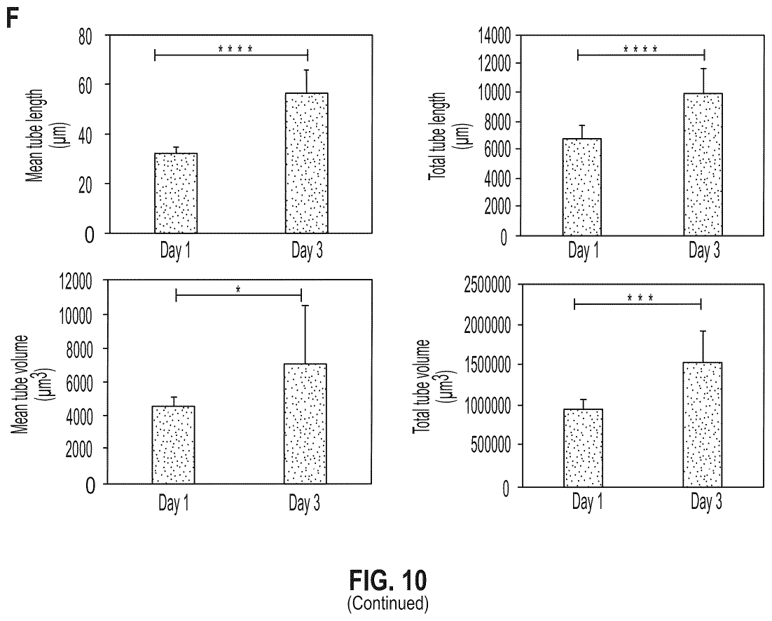

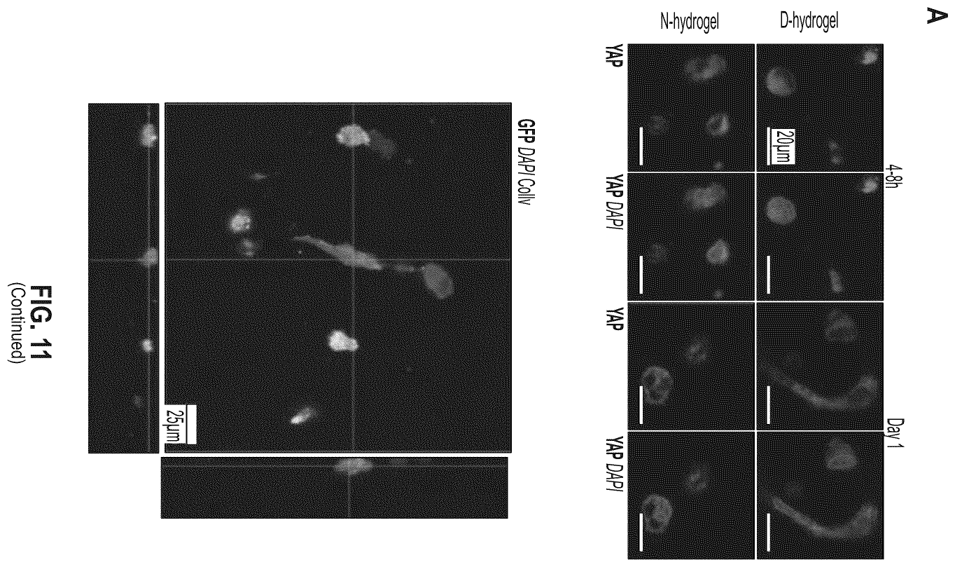

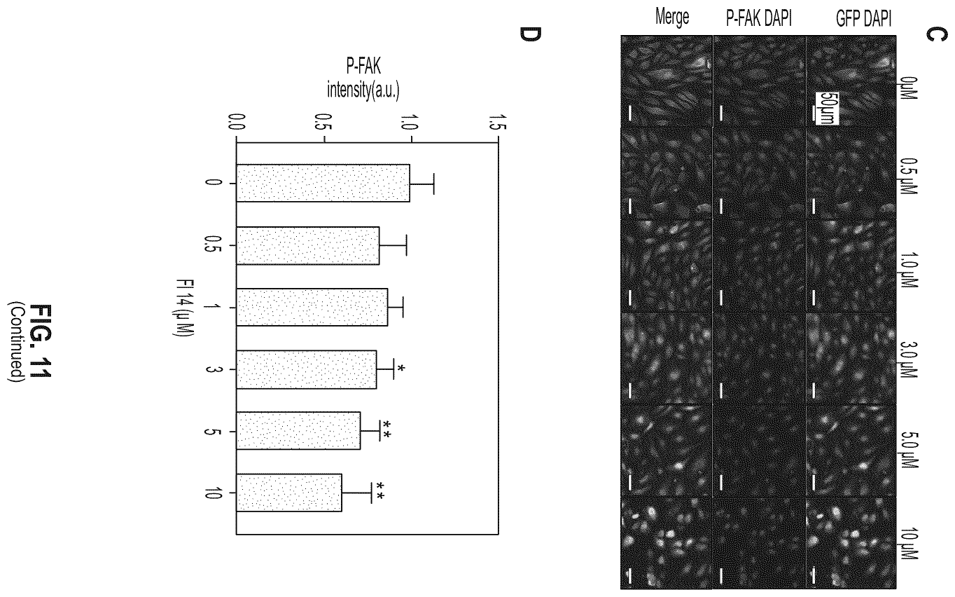

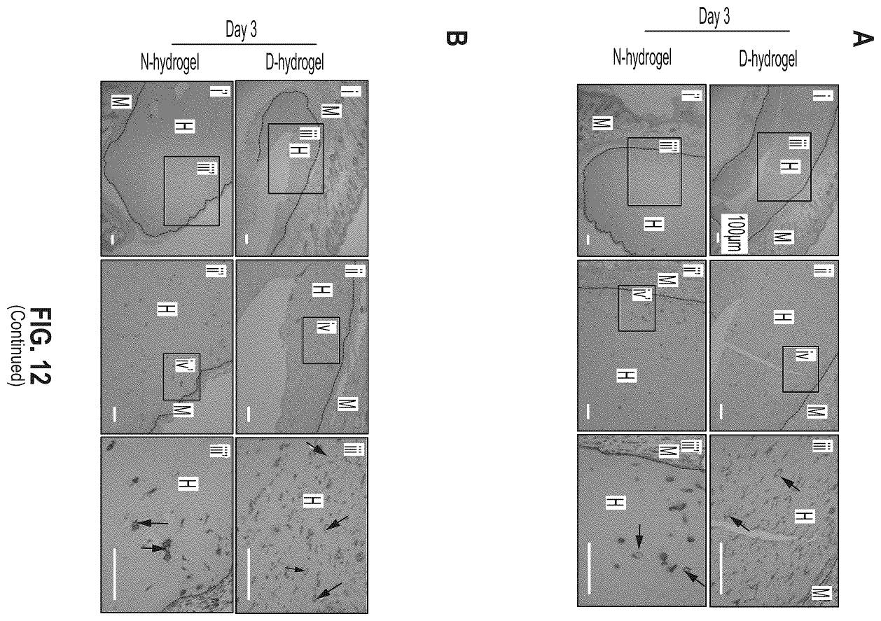

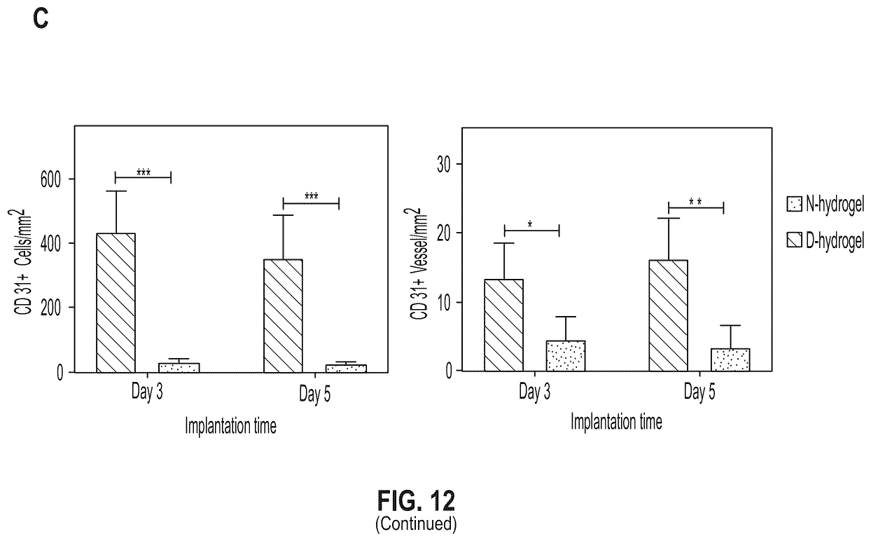

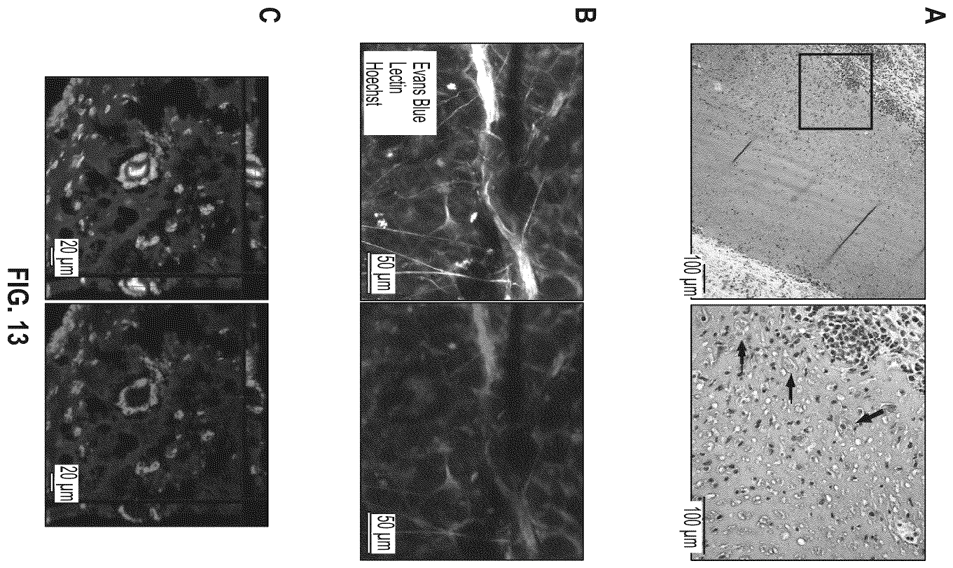

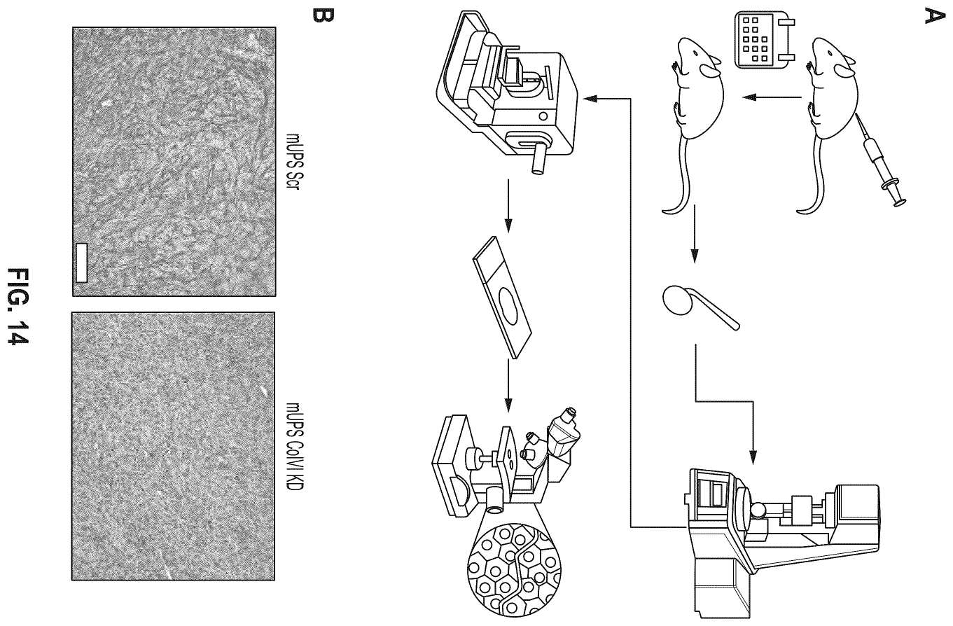

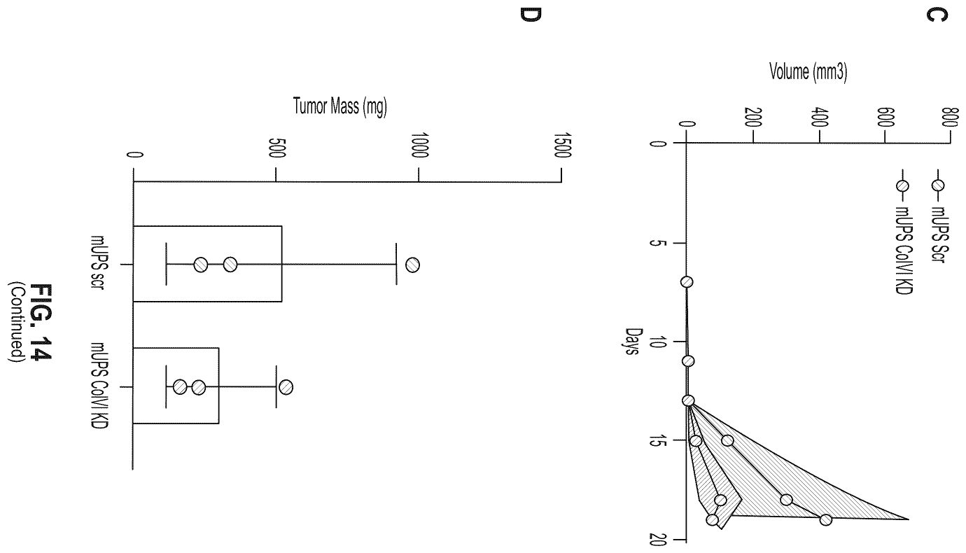

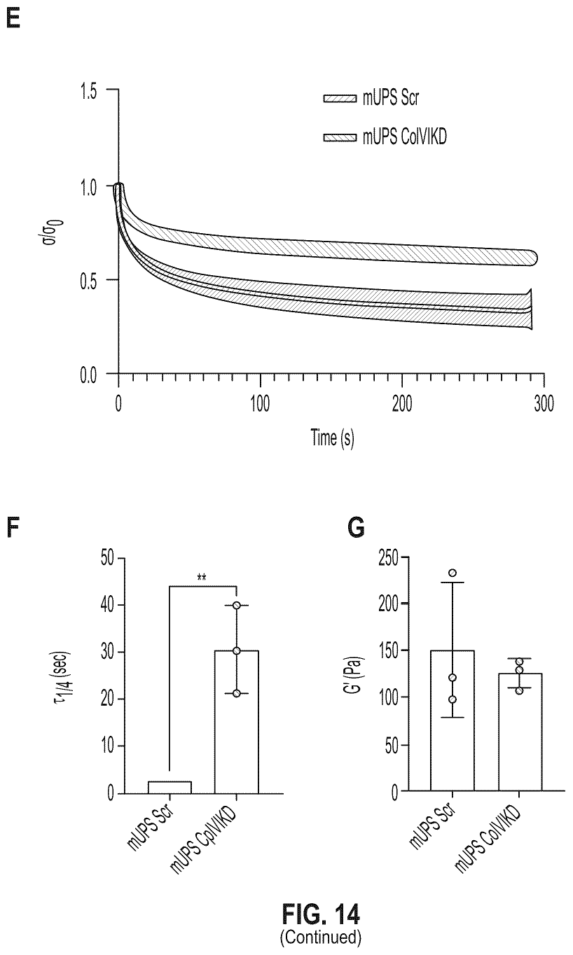

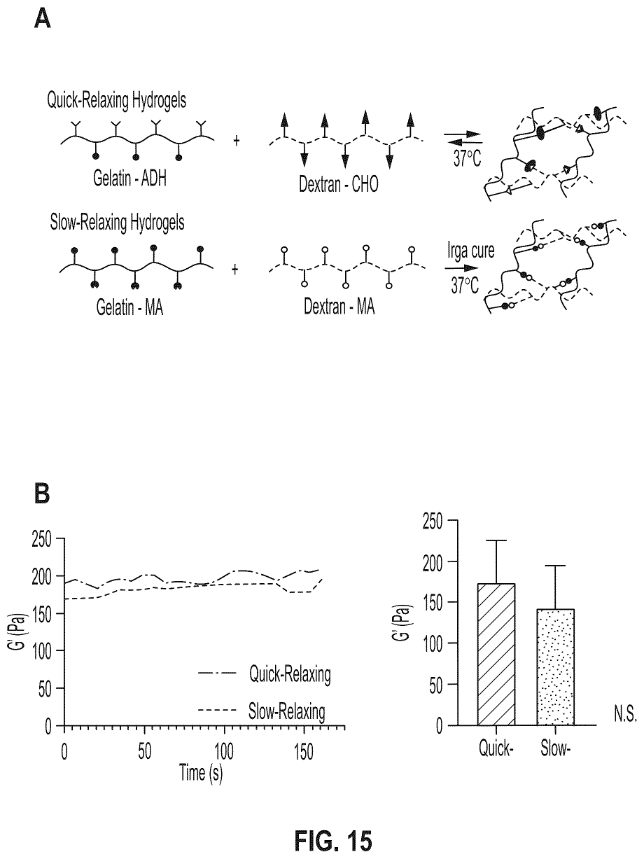

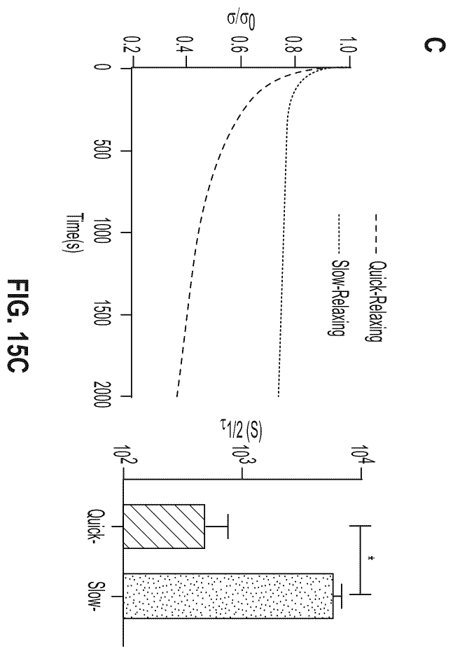

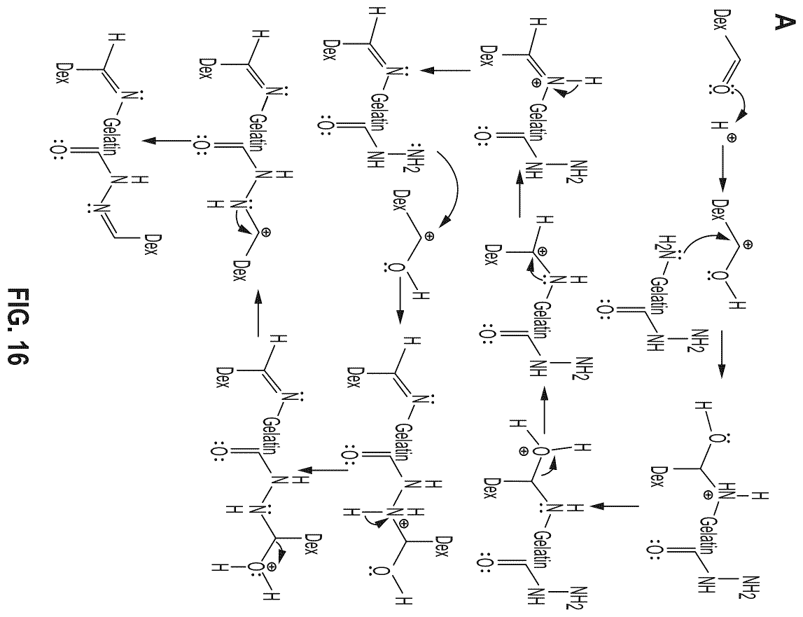

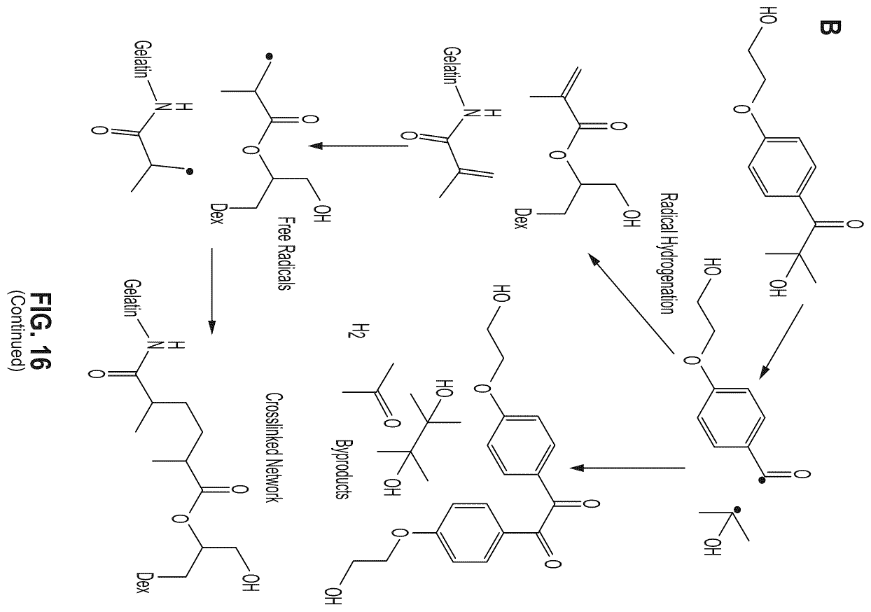

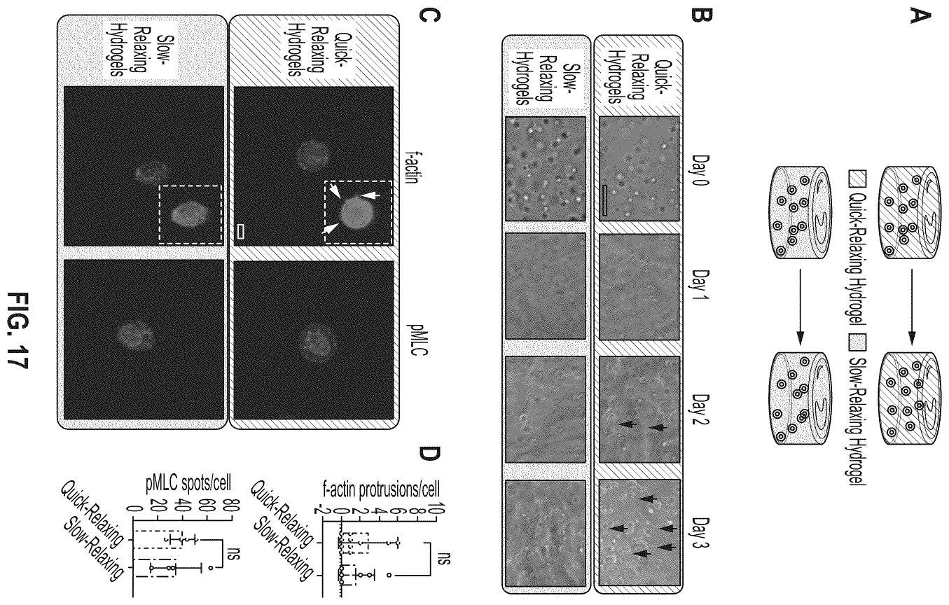

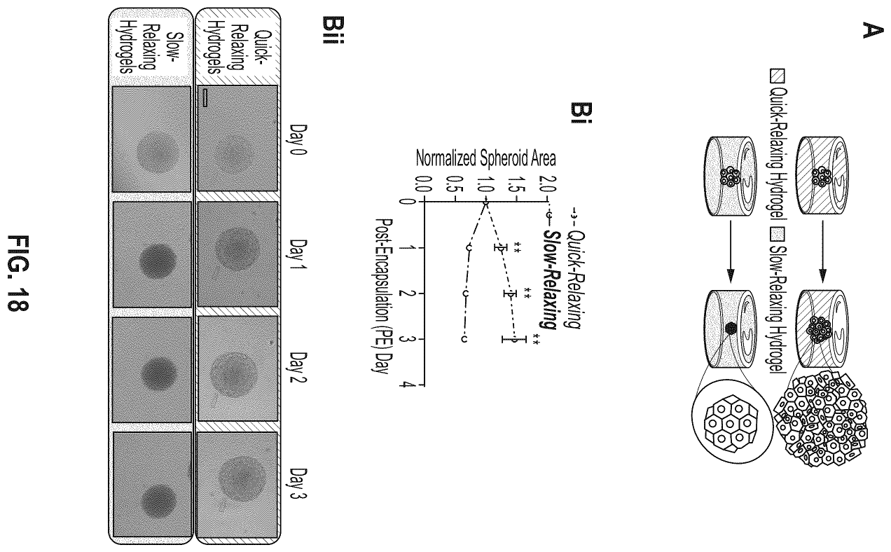

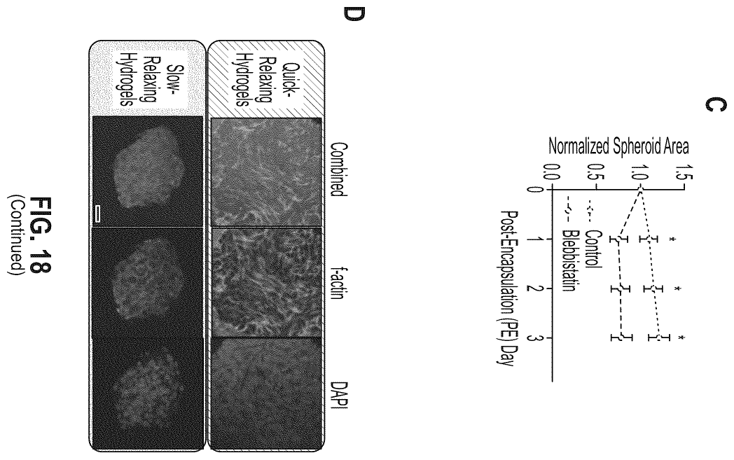

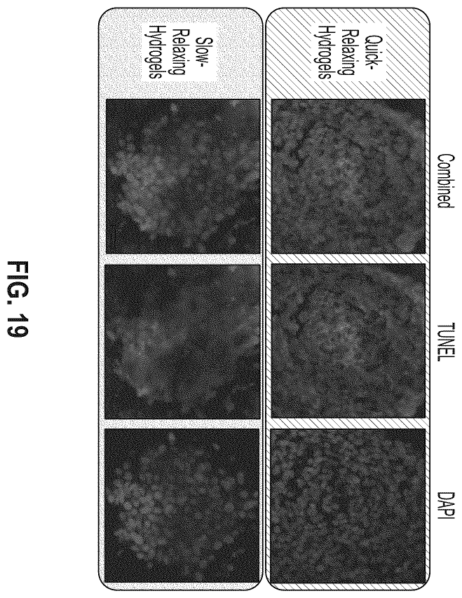

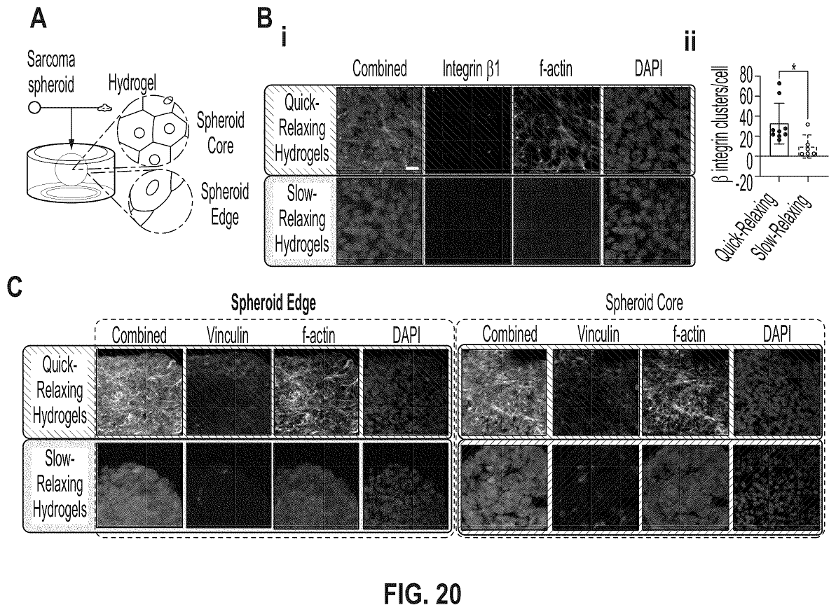

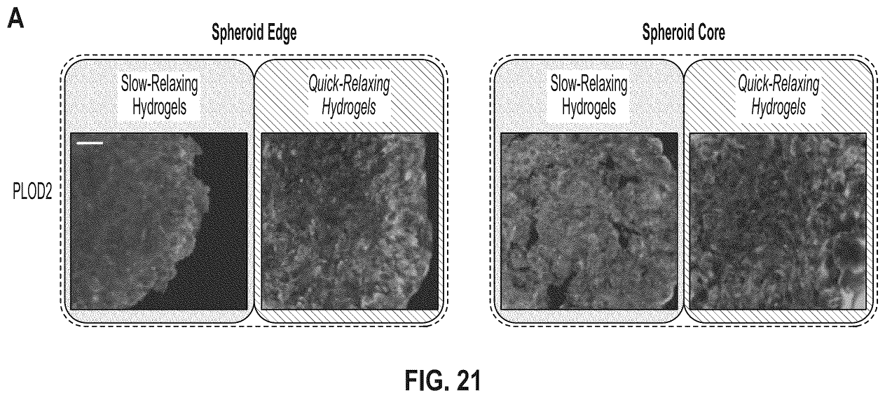

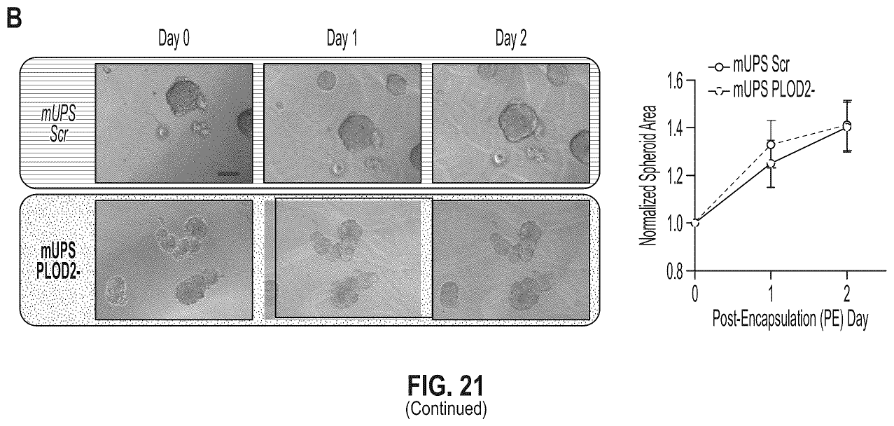

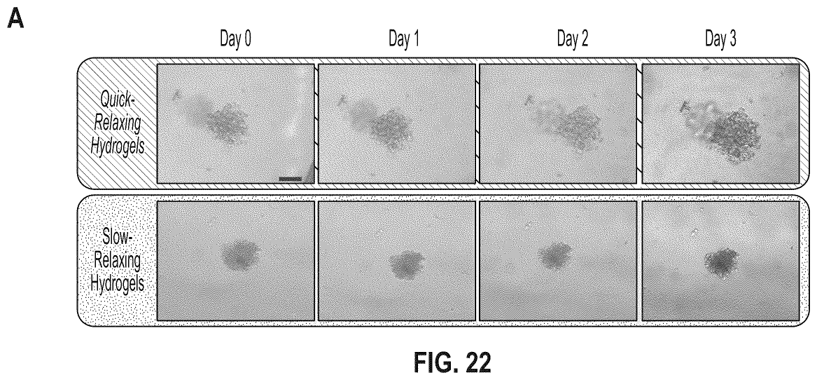

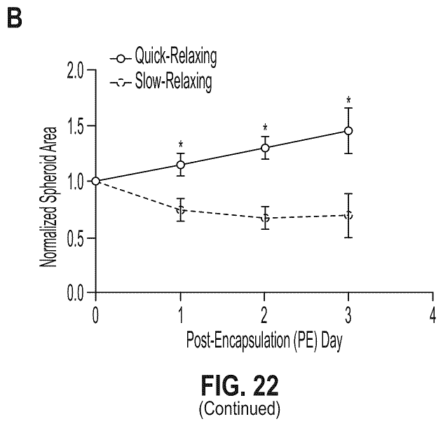

(A) shows stiff gels (˜800 kPa; as shown in FIG. 1b) limit vasculogenesis of encapsulated ECFCs. Scale bars are 100 μm.
(B) shows the diffusion rates of different time points of D-hydrogels and N-hydrogels immersed in Rhodamine B solution (1.0 mg L⁻¹) at 37° C.
(C) depicts light micrographic images showing phenotype changes of encapsulated ECFCs in D-hydrogels and N-hydrogels which were directly prepared in EGM-2 culture media. Scale bars are 100 μm.
(D) shows cell viability of the gelation precursors of D-hydrogels and N-hydrogels using WST-1.
(E) depicts live/dead staining of ECFCs encapsulated in the D-hydrogels and N-hydrogels. Scale bars are 100 μm.
(F) depicts the morphogenesis of ECFCs cultured in D-hydrogels treated with 0.5 wt % Irgacure 2959 and 50 s UV radiation.

FIG. 9 shows vacuole formation, Cdc42 expression, and ECFC networks in D- and N-hydrogels. Significance levels were set at n.s. p>0.05, *p≤0.05, **p≤0.01.

(A) shows quantification of vacuoles formed in D-hydrogels and N-hydrogels after 4-8 hrs in culture shows no difference between the two hydrogel systems (N=3 biological replicates with 5-6 images per replicate).
(B) depicts representative immunofluorescence (IF) images and corresponding quantification (n=30 cells from biological triplicates) of Cdc42 expression in ECFCs encapsulated in D-hydrogels and N-hydrogels show no differences after 4-8 hrs in culture (Cdc42 in red, nuclei in blue). Scale bars are 50 μm.
(C) depicts representative IF images of ECFCs encapsulated within D-hydrogels and N-hydrogels stained for Cdc42 after 1 day in culture (Cdc42 in red, F-actin in purple, and nuclei in blue). Scale bars are 50 μm.
(D) shows confocal projection images of vascular phenotypes in D-hydrogels and N-hydrogels on day 3 and day 5 (GFP-ECFC in green) showing extensive vascular bed formed in D-hydrogels. Scale bar is 100 μm.
(E) shows quantitative analysis of vascular tube formation on day 3 and day 5 shows mean tube length and tube volume (analysis using Imaris Filament Tracer).

FIG. 10 shows vasculogenesis, integrin expression and ECM interaction in N-hydrogels. Significance levels are set at *p≤ 0.05, ***p≤0.001 and ****p≤0.0001.

(A) shows maximum intensity projections of confocal images show development of filopodia and therefore stronger interaction of the cells with the ECM after 3 days in culture of cells embedded in the N-hydrogel (phalloidin in magenta and DAPI in blue).
(B) shows RT-PCR analysis of integrin β1 expression reveals only minimal increase of integrin expression in N-hydrogels after 3 days compared to 1 day. Further integrin expression remains lower compared to D-hydrogels.
(C) depicts confocal images of integrin staining on the cell surface of cells in N-hydrogels.
(D) shows there is no significant change in integrin β1 clustering in N-hydrogels after 3 days (n=8 images per sample from biological triplicates).
(E) depicts representative IF images of GFP-ECFCs encapsulated in D-hydrogels and N-hydrogels stained for vinculin and F-actin after 1 day in culture (GFP in green, vinculin in red, F-actin in purple and nuclei in blue). Scale bars are 20 μm.
(F) depicts vessel analysis of N-hydrogels after 1 Day and 3 Day of culture reveal only minimal increase in vascular length and complexity (n=3 biological triplicates).

FIG. 11 shows YAP localization in D- and N-hydrogels, ECM in N-hydrogels and ECFCs treated with FI 14. Significance levels were set at *p≤0.05, and **p≤0.01.

(A) depicts representative IF images of ECFCs encapsulated in D-hydrogels and N-hydrogels for 4-8 hrs and day 1 in culture, showing cytoplasmic YAP localization in both hydrogels (YAP in red, nuclei in blue). Scale bars are 20 μm.
(B) shows representative confocal maximum intensity projection with orthogonal views (on the bottom and right side of the image) of ColIV stains (in white/red; cells in green; nuclei in blue) after 3 days in N-hydrogels show no lumen formation nor ColIV localization at the basement membrane after 3 days in culture. Scale bar is 25 μm.
(C) depicts representative IF images of 2D cultured GFP-ECFCs stained with P-FAK after 1 day in culture (GFP s in green, P-FAK in red and nuclei in blue). Scale bars are 50 μm.
(D) shows quantifications of the normalized intensities of GFP-ECFCs from P-FAK staining.

FIG. 12 shows dynamic networks accelerate vasculogenesis in vivo. Significance levels were set at **p≤0.01, ***p≤0.001 and ****p≤0.0001.

(A) shows GFP-ECFC-loaded D- and N-hydrogels were directly implanted subcutaneously in nude mice and retrieved after (a) day 3 (n=3). Subpanel i and i′ are representative confocal images showing GFP-ECFC (in green) of the corresponding extracted hydrogels. Subpanel i-iv and i′-iv′ are representative histological images of CD31⁺ cells, endothelial lining in microvessels (indicated by arrows). Subpanel iii and iii′ are high magnification of correlated box in panel ii, subpanel iv and iv′ are high magnification of correlated box in panel iii.
(B) shows GFP-ECFC-loaded D- and N-hydrogels were directly implanted subcutaneously in nude mice and retrieved after day 5 (n=3). Subpanel i and i′ are representative confocal images showing GFP-ECFC (in green) of the corresponding extracted hydrogels. Subpanel i-iv and i′-iv′ are representative histological images of CD31⁺ cells, endothelial lining in microvessels (indicated by arrows). Subpanel iii and iii′ are high magnification of correlated box in panel ii, subpanel iv and iv′ are high magnification of correlated box in panel iii.
(C) shows quantification of the CD31⁺ cell densities in D- and N-hydrogels on day 3 and day 5, and quantification of the CD31⁺ microvessel densities in D- and N-hydrogels on day 3 and day 5.

FIG. 13 depicts angiogenesis into dynamic networks are functional in vivo. Acellular D- and N-hydrogels containing SDF1α (100 ng/ml) were implanted subcutaneously in C57BL/6J mice and retrieved after 3 days (n=3) following administration of intravenous Evans blue dye to label perfused vessels.

(A) depicts representative H&E image of vessels infiltrating the D-hydrogel containing red blood cells (indicated by arrows). Box in the left image is the high magnification image on the right. Scale bars are 100 μm.
(B) depicts representative confocal image of vessels infiltrating into the D-hydrogel perfused with Evans blue dye stained with lectin to localize endothelial cells (Evans blue in white, lectin in red, and nuclei in blue). Scale bars are 50 μm.
(C) depicts representative confocal maximum intensity projection with orthogonal views (on the upper and right side of the image) of luminal vessels (stained with lectin in red) infiltrating into the D-hydrogel perfused with Evans blue dye (in white and nuclei in blue). On the right are the same images without the Evans blue showing the extent of perfusion into the hydrogel.

FIG. 14 shows that the absence of collagen VI lengthens tumor stress relaxation time. (A) depicts an experimental schematic of tumor rheology and subsequent analysis. (B) depicts IHC staining for collagen VI. (C) depicts tumor growth curves for mUPS HIF1α- and Scr tumors (N=3). (D) depicts final tumor mass (N=3). (E) depicts normalized stress relaxation curves (N=3). (F) depicts stress relaxation quarter-time (_{T}1/4) (N=3). (G) depicts stiffness (N=3). Significance level is set at **p<0.01 for a t-test.

FIG. 15 shows D- and N-hydrogel networks exhibit similar stiffness and differing relaxation times. (A) is a schematic depicting networks of quick-relaxing hydrogels, made of Gel-ADH and Dex-CHO, crosslinked by imine and acylhydrazone bonds and slow-relaxing hydrogels, made of Gel-MA and Dex-MA, crosslinked by covalent bonds. (B) shows the average storage moduli (G′) of Quick-and Slow-relaxing hydrogels (N=4). (C) depicts representative stress relaxation curves of D- and N-hydrogels and quantification of timescale when stress is relaxed to half of its original value, T_{1/2}, from stress relaxation tests (N=4).

FIG. 16 depicts exemplary crosslinking chemistry for (A) quick-relaxing hydrogels and (B) slow-relaxing hydrogels.

FIG. 17 shows sarcoma cells encapsulated in quick-relaxing hydrogels have a round morphology with filopodia-like protrusions. (A) shows a graphical schematic of sarcoma spheroids encapsulated in quick- and slow-relaxing hydrogels. (B) depicts light micrographic images showing differences in single-cell sarcoma cells over three days PE (ii). Orange arrows indicate beginning matrix interactions and protrusions PE Day 2. Yellow arrows indicate mature filopodia and lamellipodia on PE Day 3. Scale bar is 100 μm. (C) depicts confocal microscopy maximum intensity projections of f-actin (red), myosin-II (red, and DAPI (blue) in cells on PE Day 2. The insert shows enhanced contrast. Arrows indicate filopodia-like protrusions. Scale bar is 5 μm. (D) depicts f-actin clusters/cell (N=2, n=7-13) (i) and pMLC spots/cell (N=1, n=4) (ii).

FIG. 18 shows quick and slow relaxing hydrogels modulate sarcoma spheroid growth. (A) shows a graphical schematic of spheroids encapsulated in quick- and slow-relaxing hydrogels. (B) depicts light micrographic images showing quantified differences (ii) in mUPS spheroid size across 3 days in culture (i) normalized to Day 0 (N=9) (ii). Scale bar: 100 μm. (C) shows quantified differences in mUPS spheroids in blebbistatin and control hydrogels (N=9). (D) shows confocal microscopy maximum intensity projections of f-actin (green) and DAPI (blue) in spheroids at Day 3. Scale bar: 20 μm. Significance levels are set at *p<0.05, ** p<0.01 for a t-test.

FIG. 19 shows mUPS spheroids encapsulated in quick-and slow-relaxing hydrogels do not exhibit differences in apoptosis. Confocal microscopy maximum intensity projections of TUNEL (green) and DAPI (blue) at Day 3. Scale bar: 20 μm.

FIG. 20 shows quick-relaxing hydrogels promote increased focal adhesion formation in sarcoma spheroids. (A) shows a experimental schematic of spheroid core and edge. (B) depicts confocal microscopy MIP of integrin β1 (green) and f-actin (white) within the spheroid core at Day 1 (i), and subsequent quantification (ii) (N=6-9). Scale bar: 10 μm. (C) depicts confocal microscopy maximum intensity projections (MIP) of vinculin (red), f-actin (white), and DAPI (blue) at the spheroid edge and within the spheroid core at Day 1. Scale bar: 20 μm.

FIG. 21 show PLOD2 is not responsible for spheroid growth in quick-relaxing hydrogels. (A) shows confocal microscopy maximum intensity projections (MIP) of PLOD2 (green) at the spheroid edge and within the spheroid core at Day 1. Scale bar: 20 μm. (B) depicts light micrographic images (i) of KIA Scr and PLOD2-spheroids in quick-relaxing hydrogels across 2 days of culture and subsequent quantification (ii) (N=18). Scale bar: 100 μm.

FIG. 22 depicts HT 1080 spheroids grow in quick-relaxing hydrogels, and shrink in slow-relaxing ones. (A) depicts light micrographic images of KIA Scr and PLOD2-spheroids in quick-relaxing hydrogels across 2 days of culture. (B) depicts subsequent quantification of the spheroids (N=10).

### DETAIL DESCRIPTION OF THE INVENTION

The following description is provided to enable any person skilled in the art to make and use the invention. Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. Various modifications, however, will remain readily apparent to those skilled in the art, since the general principles of the present invention have been defined herein specifically to provide one example of one application of the invention. All references cited herein are incorporated by reference as if each had been individually incorporated.

Mechano-sensing has emerged as an important regulator of cellular behaviors, thus affecting the design of hydrogels for tissue engineering. Specifically, it is well established that the secretion of matrix metallopeptidases (MMPs) to allow matrix degradation is a crucial step in vascular morphogenesis (Chun et al., 2004; Davis and Senger, 2005; Iruela-Arispe and Davis, 2009; Sacharidou et al., 2010; Stratman et al., 2009). In agreement, studies focusing on vascular engineering in hydrogel matrices have shown that progression of vascular morphogenesis occurs alongside decreasing hydrogel stiffness through the activation of MMPs (Ghajar et al., 2006; Hanjaya-Putra et al., 2011; Shen et al., 2014; Turturro et al., 2013). For example, Moon et al. (2010) developed protease-sensitive poly(ethylene glycol) (PEG) hydrogels with selected moduli for blood vessel formation from human umbilical vein endothelial cells (ECs). Beamish et al. (2019) engineered another PEG-based hydrogel with MMP-sensitive crosslinkers for capillary morphogenesis from encapsulated ECs. These studies have revealed that with a suitable stiffness, the robust in vitro vascular bed could be engineered in the synthetic elastic hydrogels. However, independent of matrix stiffness, recent studies have implicated that viscoelasticity can guide cellular behaviors in hydrogels (Brown et al., 2018; Chang and Chaudhuri, 2019; Lewis et al., 2019; Lou et al., 2018; Nam et al., 2019; Wang and Heilshorn, 2015). Unlike elastic hydrogels, covalently crosslinked viscoelastic hydrogel networks formed with reversible physical interactions or dynamic covalent bonds (de Greef and Meijer, 2008; Wei et al., 2014, 2017; Wojtecki et al., 2011) are able to reconstitute in response to external deformation in a process known as stress relaxation (Carreau, 1972; Matsuoka, 1992). The stress relaxation performance of these hydrogels has been found to be consistent with soft tissues and naturally derived extracellular matrix (ECM) and thus can be used to better mimic the mechanics of the native microenvironments of cells (Chaudhuri et al., 2016; Geerligs et al., 2008; Levental et al., 2007; Liu and Bilston, 2000; McDonald et al., 2009). These dynamic hydrogel (D-hydrogel also referred to herein as quick-relaxing hydrogel) networks are able to be remodeled and rearranged in response to the traction forces imposed by the encapsulated cells during culture, allowing the cells to respond in physiologically relevant modes (Huebsch et al., 2010; Swift et al., 2013; Trappmann et al., 2012).

A plethora of strategies have been developed to synthesize hydrogels with stress-relaxing behaviors to track the cell behavior and fate, including spreading, proliferation, and differentiation of mesenchymal stem cells (MSCs) (Brown et al., 2018; Chaudhuri et al., 2016; Tang et al., 2018), fibroblasts (Chaudhuri et al., 2015), myoblasts (McKinnon et al., 2014), and neural progenitor cells (Madl et al., 2017), as well as the motility and mode of the migration of cancer cells (Lewis et al., 2017, 2019). Nonetheless, studies of how hydrogel network dynamics regulate the assembly of a tissue are scarce and there is a need to develop D-hydrogel systems in which these dynamics can be studied and the mechanisms delineated. Moreover, while the regulation of the transcriptional regulator YAP (Yes-associated protein) has been documented (Dupont et al., 2011), little is known about the underlying signaling pathways that mediate this mechano-sensing process of cellular interactions with hydrogel networks throughout tissue formation.

In one aspect, present invention relates to D-hydrogel networks that activate a distinctive mechano-sensing matrix remodeling mechanism that allows vascular tissue assembly. To investigate this, the inventors engineered a viscoelastic hydrogel system with dynamic covalent crosslinks that permit vascular tissue assembly, enabling the inventors to determine the role of dynamic networks and the underlying mechanism in regulating vascular tissue morphogenesis. As used herein, “dynamic covalent crosslinks” refers to hydrogel crosslinking groups wherein the covalent bonds of the crosslinking groups break and reform in response to deformation. Using these D-hydrogels, the present disclosure shows that network dynamics enable integrin clustering via increased cell contractility, leading to the recruitment of vinculin and the formation of large focal adhesions (FAs), thus allowing rapid network formation, while non-dynamic matrices prevent the clustering of integrins and subsequently the initiation and progression of vascular bed formation. Matrix dynamics are necessary for integrin clustering and activation highlighting the importance of inside-out signaling through integrin and vascular morphogenesis.

Matrix dynamics influence how individual cells develop into complex multicellular tissues. In an aspect, the present invention relates to hydrogels with identical polymer components but different crosslinking capacities to enable the investigation and exploitation of mechanisms underlying vascular morphogenesis. It is shown that dynamic (D) hydrogels increase the contractility of human endothelial colony-forming cells (hECFCs), promote the clustering of integrin β1, and promote the recruitment of vinculin, leading to the activation of focal adhesion kinase (FAK) and metalloproteinase expression. This leads to the robust assembly of vasculature and the deposition of new basement membrane. It is also shown that non-dynamic hydrogels (N-hydrogels, also referred to herein as slow-relaxing hydrogels) with similar composition but different crosslinkages do not promote FAK signaling and that stiff D- and N-hydrogels are constrained for vascular morphogenesis. Furthermore, D-hydrogels promote hECFC microvessel formation and angiogenesis in vivo. The results indicate that cell contractility mediates integrin signaling via inside-out signaling and emphasizes the importance of matrix dynamics in vascular tissue formation, thus providing vascularization and tissue engineering applications.

The inventors designed hydrogels from polypeptides and polysaccharides. Polypeptides for use in hydrogels of the present invention include gelatin, soy protein, albumin, and collagen. In embodiments, the polypeptides include RGD and MMP sensitive segments. Exemplary polypeptides are water soluble and/or contain free amino groups for further chemical modification as described herein. Polysaccharides suitable for use in hydrogels of the present invention include hyaluronic acid, agarose, alginate, cellulose and derivatives thereof (e.g., methylcellulose), chitosan, and dextran. Gelatin, a polypeptide, and dextran, a polysaccharide, are two bio-polymers commonly used for tissue engineering (Blatchley et al., 2015; Kang et al., 1999; Sun et al., 2010) and are used herein to examine the role of matrix dynamics on tissue assembly in the hydrogels of the present invention. Gelatin was selected as the main backbone of the hydrogel for its bio-functional cell adhesive motif and cell-mediated MMP proteolytic degradable sites on its polymer chains, both required for vascular morphogenesis (Blatchley et al., 2019; Park and Gerecht, 2014; Wei and Gerecht, 2018), although other proteins can also be used. Dynamic covalent bonds, for example, crosslinks formed by reacting imine and acylhydrazone, are utilized to generate dynamic network hydrogels. Other cross-linking groups used to prepare stress-relax or self-healing hydrogels may also be used. Static covalent bonds, for example crosslinks formed by reacting acrylates or methacrylates (MAs), are utilized to form static, non-dynamic network hydrogels as controls (FIG. 1A). For D-hydrogel network formation, the gelatin (Gtn) was modified with adipic acid dihydrazide (ADH) to obtain the Gtn-ADH (FIG. 1H), and the dextran was oxidized to generate aldehyde-modified dextran (Dex-CHO) (FIG. 1A). The in situ formation of the D-hydrogel was achieved by homogeneously mixing Gtn-ADH and Dex-CHO in phosphate-buffered saline (PBS, pH 7.4) under physiological conditions (37° C.) with a fixed Gtn-ADH concentration of 5.0 wt %. The imine and acylhydrazone bonds were cross-linked by aldehyde groups on Dex-CHO with the original amino groups and modified acylhydrazide groups on Gtn-ADH, respectively. Known as dynamic covalent bonds, both imine and acylhydrazone have been used for the preparation of numerous adaptable and self-healing hydrogels, which can perform an intrinsic dynamic equilibrium of bond association and dissociation in polymer networks under physiological conditions (Wei et al., 2014, 2015). Moreover, the acylhydrazone bonds have been shown to be more stable than imine bonds (Wei et al., 2015), and thus can further accelerate the crosslinking efficiency and increase the stability of the D-hydrogel networks. Rheological analysis found that the crosslinking point of the storage modulus (G′) and loss modulus (G′″), which presents an estimate of gelation time, occurs much faster in Gtn-ADH than Gtn with the higher final G′ (FIG. 1K and 1L). These data were in concordance with the gelation kinetics observed by phase transition (FIG. 1M), demonstrating that the acylhydrazone bonds induce rapid network formation and enhance the stability of the hydrogels. For the non-dynamic hydrogel (N-hydrogel), MA-modified gelatin (Gtn-MA) (FIG. 1I) and glycidyl MA modified dextran (Dex-GMA) (FIG. 1J) were conventionally crosslinked by UV polymerization in PBS (pH 7.4) to form static covalent hydrogel networks (FIG. 1A). The concentrations of the backbones of both hydrogels were kept identical and constant throughout the experiments.

D-hydrogels and N-hydrogels were fabricated with similar initial elastic moduli by adjusting the crosslinker concentration of Dex-CHO (for D-hydrogel) and UV crosslinking time (for N-hydrogel), respectively (stiff or soft; FIG. 1B). By varying the amount of available cross-linkages, the stiffness or softness of the D-hydrogel can be controlled. When preparing a hydrogel with excess Gtn-ADH, the stiffness of the hydrogel can be increased by increasing the concentration of Dex-CHO. Likewise, the softness of the hydrogel can be increased by decreasing the concentration of Dex-CHO. For N-hydrogels, the stiffness or softness is controlled by varying the amount of UV crosslinking time. For stiffer hydrogels the UV crosslinking time is increased, whereas for softer hydrogels the UV crosslinking time is decreased. A stiff hydrogel is firmer, and less susceptible to deformation, whereas soft hydrogels are more malleable and more susceptible to deformation. In the context of the present invention stiff hydrogels have a shear storage moduli of greater than about 400 Pa, for example about 600 Pa, and soft hydrogels have a shear storage moduli of less than about 400 Pa, for example about 200 Pa. To evaluate the rate of the stress relaxation profiles of these hydrogels, a constant initial strain was applied in rheological time sweep tests. The resultant relaxation curves were obtained after normalizing the relaxed stresses to the initial stresses (FIG. 1C). The corresponding relaxation rate was quantified as time of the initial stress to half its original value (T_{1/2}). It was found that the stress relaxation rates decrease with the increasing stiffness of D-hydrogels (FIG. 1D, 1N, and 1O). In N-hydrogels, the relaxation rates are much slower than the D-hydrogels, in both soft and stiff conditions, due to their non-dynamic covalent crosslinks. Finally, the soft D-hydrogels and N-hydrogels immersed in culture media and incubated at 37° C. for 3 days to assess whether their viscoelasticity would be influenced or changed by swelling. As seen in FIG. 1E, there was no significant difference in the G′ of both D- and N-hydrogels after 3 days of incubation in media. However, the stress relaxation rates of D-hydrogels decreased with swelling time (FIG. 1F and 1G). Nonetheless, the T_{1/2} of D-hydrogels were still significantly shorter than the corresponding N-hydrogels on days 2 and 3 (FIG. 1G), thus providing a platform to investigate the role of hydrogel network dynamics during vascular morphogenesis.

Also examined is the impact of stress relaxation time on sarcoma spheroid growth and single-cell matrix interactions to investigate stress relaxation resulting from sarcoma cells. Specifically, the inventors leverage the gelatin-/dextran-based hydrogel system with consistent stiffness and differing stress relaxation times and couple immunofluorescent (IF) and light microscopy to visualize cell-matrix interactions.

### Hydrogels with Dynamic Networks Promote Rapid Vascular Morphogenesis

Vascular morphogenesis in hydrogels initiates when encapsulated ECs engage integrins to interact with the matrix, followed by the formation of void spaces called vacuoles that then coalesce intercellularly and intracellularly to form a lumen (Bayless et al., 2000; Crosby and Zoldan, 2019; Davis and Bayless, 2003; Davis and Camarillo, 1996; Hanjaya-Putra et al., 2011). Sprouting and branching, concurrent with matrix degradation and remodeling, conclude in the formation of a perfusable, nascent vasculature (Davis and Bayless, 2003; Davis and Camarillo, 1996) (FIG. 2A).

Toward the generation of a vascular bed, human endothelial colony-forming cells (ECFCs) were encapsulated in the D-hydrogel and N-hydrogel and to examine the kinetics of morphogenesis. Encapsulating ECFCs in the stiffer hydrogels (˜600 Pa) caused the slow progression of vascular morphogenesis (FIG. 8A), corroborating previous works using a variety of hydrogels (Bordeleau et al., 2017; Brown et al., 2020; Hanjaya-Putra et al., 2011; McCoy et al., 2016; Park and Gerecht, 2014; Zheng et al., 2012). Using softer hydrogels (˜200 Pa), vacuoles could be observed forming in both hydrogels within 4-8 h of encapsulation. Sprouting and branching were observed in the D-hydrogels on day 1, expanded by day 2, and formed a complex vascular bed by day 3 of culture. In contrast, sprouting and branching events were observed in the N-hydrogels only on day 2 following encapsulation and expanded by day 3 (FIG. 2B). Next it was examined whether the difference in phenotype may be due to different diffusion rates of nutrients and cytokines/growth factors in the two hydrogels. Using Rhodamine B dye, it was confirmed that there was no significant difference in diffusion rates between D- and N-hydrogels (FIG. 8B), demonstrating similar transport properties of the hydrogel networks. To further eliminate the possible concerns of diffusion, both hydrogels were directly prepared with the endothelial growth medium-2 (EGM-2) media. Here, too, the ECFCs in D-hydrogels still experienced faster morphogenesis from days 1 to 3 when compared with N-hydrogels (FIG. 8C).

While multiple studies have demonstrated the safety and cell-based formation of vasculature by UV crosslinked hydrogels (Beamish et al., 2019; Chen et al., 2012; Moon et al., 2010), the inventors examined and compared the cytotoxicity of D-hydrogels and N-hydrogels to eliminate the possible impact on ECFCs. No significant change in cell viability was observed in samples treated with various gelation precursors of D- and N-hydrogels and UV irradiation, when compared with PBS controls (FIG. 8D). Moreover, live/dead staining of ECFCs encapsulated in the D-hydrogel and N-hydrogel for 12 h further showed comparable cytocompatibility (78.21% versus 84.83%, respectively; FIG. 8E). Finally, ECFC morphogenesis was found to progress along the 3 days in culture when encapsulated in D-hydrogels with additional Irgacure 2959 and UV irradiation (FIG. 8F).

Examination of vacuole formation in both D- and N-hydrogels found no significant differences (FIG. 9A and 9B). Previous works have demonstrated that lumen formation is mediated through the activation of the Rho guanosine triphosphatase (GTPase) Cdc42 within 4-24 h, which is modulated with matrix stiffness (Hanjaya-Putra et al., 2010; Kim et al., 2014). When examining Cdc42 expression, no significant difference was found between the two hydrogels up to 24 h (FIG. 9C), suggesting that hydrogel network dynamics do not regulate the early stages of vacuole and lumen formation. As vascular morphogenesis progresses with decreasing hydrogel stiffness (Hanjaya-Putra et al., 2011; Shen et al., 2014), next examined were changes in stiffness and stress relaxation along the progression of vasculo-genesis. It was found that the stiffness of both hydrogels decreased along the culture period, with D-hydrogels becoming significantly softer than N-hydrogels on days 2 and 3 of culture (FIG. 2C), suggesting that the dynamic networks are being degraded as vascular morphogenesis is progressing. Stress relaxation behaviors of both the D-hydrogels and N-hydrogels were not influenced by the encapsulated cells and were consistent with the results of acellular hydrogel incubation in media during the duration of the experiment (FIG. 2D). Finally, the characteristics of the vascular bed formed at the end of the morphogenesis were compared. It was found that the dynamic networks enable longer and thicker vessels (tube length and tube volume), as well as the formation of open lumens compared with the non-dynamic matrix (FIG. 2E-2I). In addition, no significant differences were found in vasculature characteristics between days 3 and 5 of culture following encapsulation of the ECFCs in D- and N-hydrogels (FIG. 9D and 9E). Thus, hereafter analysis focuses on culture periods up to 3 days.

### Hydrogels with Dynamic Networks Promote FA Formation and Integrin Clustering

As no difference was found in the kinetics of vacuoles, next explored was the role of hydrogel networks on cell sprouting and branching, a critical step in the formation of cohesive tissue structures. It was hypothesized that the relaxed stress of dynamic networks allows integrins to better engage with hydrogel adhesion motives, such as RGD. The larger network dynamic and stress relaxation of the D-hydrogels further allows the efficient formation of initial Focal Adhesions (FAs) and cell contraction, leading to the formation of larger integrin clusters. This in turn leads to an increase in integrin signaling and cell contraction and allows cells to deform and remodel the hydrogel networks, thereby enabling efficient sprouting and branching.

To investigate the physical interactions between ECFCs and polymer networks of D-hydrogels or N-hydrogels, 3D traction force microscopy was performed (Legant et al., 2010; Yoon et al., 2019). The fluorescent beads were encapsulated with ECFCs in both D-hydrogel and N-hydrogels, and the displacement and speed of the beads with respect to cells were analyzed, thus measuring forces applied by ECFCs to the surrounding hydrogel networks. It was found that the ECFCs encapsulated in the D-hydrogel deformed the polymer networks more than in the N-hydrogels, as indicated by the significantly longer track displacement and higher speed of bead movements during the time lapse (FIG. 3A). To further understand the underlying changes of the differences in traction force, the inventors analyzed phosphorylated myosin light chain (pMLC). It was found that cells embedded in D-hydrogels demonstrate protrusions and filopodia development within 12 h of culture, with pMLC strongly colocalized to actin (FIG. 3B). However, cells embedded in N-hydrogels did not show any formation of filopodia nor the development of protrusions and localization of the cells after 12-24 h of culture, indicating no ability to migrate within the gel (FIG. 3B and 10A). In addition, pMLC is mostly localized to the nucleus of the cells embedded in N-hydrogels after 12 h of culture, with no changes in total pMLC levels in D- and N-hydrogels (FIGS. 3B and 3C). Nuclear localization was not changed after 24 h of culture; however, the overall amount of pMLCs was significantly reduced in cells embedded in N-hydrogels after 24 h of culture (FIG. 3D). Overall, these results indicate a strong contribution of matrix dynamic in the regulation of cell-ECM contacts and cell sprouting.

FAs are complex structures that localize to sites of cell-matrix interaction through which integrins and scaffold proteins link the actin cytoskeleton to the ECM (Humphries et al., 2007; Sun et al., 2016). FAs usually form at filopodia sites and protrusions functioning as adherence points during cell migration. Therefore, the inventors investigated the interaction of integrins with the hydrogel network and whether the decrease in traction force has an effect on integrin clustering and FA formation. A strong increase was found in integrin 31 levels and clustering after 24 h in culture and a significant upregulation of integrin 31 and integrin aV mRNA expression in the D-hydrogels, compared to N-hydrogels (FIG. 3E-3G). These results suggest that the D-hydrogel networks allow for easier deformation and strong traction forces, which pull on the matrix to a greater extent, facilitating the interaction of integrins with the hydrogel networks followed by integrin clustering.

This clustering of integrin 31 ultimately leads to the recruitment of vinculin (del Rio et al., 2009; Ziegler et al., 2008) and the formation of stable FAs (FIG. 3H and 3I). The formation of FA in ECFC-encapsulated D-hydrogels leads to a remodeling of the cytoskeleton and changes in cell morphology, allowing the ECFCs to spread and initiate sprouting. While ECFCs encapsulated in the N-hydrogel are still capable of vacuole formation, their weak interaction with the matrix and the absence of stable FA results in a static state of ECFCs with no spreading and sprouting (FIG. 3J).

Further analysis of the N-hydrogels and whether there is a delay in the timing of vasculogenesis showed that there is a moderate increase in integrin 31 expression as well as an increase in filopodia and micro-spikes after 3 days of culture, indicating a delay in integrin clustering (FIG. 10B-10D), FA formation (FIG. 10E), and, subsequently, vascular bed progression (FIG. 10F). As a result of the initial delay, the cells do not “catch up” with robust vascular bed development even after 5 days of culture (see FIG. 9D and 9E).

### Hydrogel Network Relaxation is Necessary for Contraction-Mediated Integrin Clustering and Subsequent Vessel Formation

Whether integrin clustering triggers outside-in signaling to facilitate integrin activation or whether clustering occurs after initial activation is still debated. However, certain studies point toward initial integrin FAK activation in an early phase, followed by contractility-mediated integrin clustering, resulting in full FA formation and signaling (Yu et al., 2011).

To further analyze the effect of hydrogel network dynamics on the initiation and progression of vasculogenesis via contractility-mediated integrin clustering, the inventors investigated whether the inhibition of cell contractility using blebbistatin has a similar effect on integrin clustering, as observed in N-hydrogels. As described above, N-hydrogels do not allow for cell contractility, therefore inhibiting the aggregation of distant integrin clusters and stable FA formation (see FIG. 4B). To further investigate this phenomenon, ECFCs embedded into D-hydrogels were treated with blebbistatin, a known inhibitor of myosin-II′s ATPase and cell contractility (Kovács et al., 2004). The inhibition of cell contraction did not affect integrin intensity and number, but did lead to a significant decrease in integrin cluster size and area covered (FIG. 4A and 4B). These indicate that cell contractility does not regulate initial integrin-RGD binding rather the aggregation and formation of larger integrin clusters. The inhibition of integrin aggregation also led to a decrease in cellular protrusions and filopodia, which is ultimately needed for active cell sprouting and vessel formation (FIG. 4C). Subsequently, the formation of the vascular bed was inhibited as shown by shorter tube length and reduced vessel volume in cells treated with blebbistatin for 3 days in D-hydrogels (FIG. 4D).

It was concluded that after the initial formation of small FAs, cell contractility via pMLC-mediated actin contraction promotes integrin clustering, the recruitment of vinculin, followed by the activation of downstream signaling events, thus promoting downstream signaling and vascular morphogenesis. The non-dynamic matrix, however, prevents myosin-mediated integrin clustering and therefore does not allow for efficient sprouting and vessel formation. Moreover, matrix rigidity seems to have a significant effect on pMLC localization and translocation to the nucleus, as shown in FIG. 3. This effect cannot be overcome by cells, even after several days in culture.

### Dynamic Networks Lead to the Activation of FA Kinase (FAK), the Upregulation of MMP Expression, and New ECM Deposition

Previous publications suggest that integrin clustering and activation occur in several phases via regulatory signals that originate within the cell cytoplasm, and are then transmitted to the external ligand-binding domain of the receptor (Yu et al., 2011). These “inside-out” signals begin with initial integrin binding to RGD sites, leading to the recruitment of paxillin and FAK in a mechanical force-independent manner (Yu et al., 2011). The recruitment of FAK to the FAs leads to its phosphorylation and its consequent activation (Ren et al., 2000; Shi and Boettiger, 2003; Wang and McNiven, 2012). This process is followed by actin polymerization and myosin activation, leading to the aggregation of distant integrin clusters and outward translocation (Yu et al., 2011). This process then leads to the recruitment of vinculin and the formation of large stable FAs, resulting in higher cell motility, which is necessary for EC migration during angiogenesis (Hosseini et al., 2019; Pedrosa et al., 2019). Due to the observed cell contractility-mediated integrin clustering and subsequent formation of FAs in the D-hydrogels, the inventors sought to investigate whether FAK is activated in this system and whether it directly contributes to integrin clustering. It was found that after 24 h post-encapsulation, ECFCs cultured in D-hydrogels show significantly higher levels of phosphorylated FAK compared with ECFCs embedded in N-hydrogels (FIG. 5A). However, although ECFCs interact more vigorously with the surrounding matrix in the D-hydrogels through a stronger formation of FAs and activation of FAK, the YAP (FIG. 11A) was not localized into the nucleus, as previously shown (Tang et al., 2018). This may be attributed to the relatively low stiffness of D- and N-hydrogels (˜200 Pa) used for ECFC culture. These findings suggest that D-hydrogel networks can support the formation of FAs and FAK phosphorylation in encapsulated ECFCs.

Furthermore, matrix remodeling and degradation is an important step toward the initiation of vasculogenesis, allowing ECs to invade and vascularize tissues (Davis and Senger, 2005; Iruela-Arispe and Davis, 2009). Specifically, membrane type 1 (MT1)-MMP, MMP1, and MMP9 play major roles in the degradation of the ECM during angiogenesis (Hanjaya-Putra et al., 2011; Park and Gerecht, 2014). Therefore, it was analyzed whether dynamic networks can promote the expression of MMPs in ECFCs.

For this, ECFCs were encapsulated in D- and N-hydrogels and cultured for 24 h, before the mRNA expression levels were analyzed by qRT-PCR. A significant increase in the expression of MT1-MMP, MMP1, and MMP9 was found in ECFCs encapsulated in D-hydrogels, compared to cells embedded in N-hydrogels (FIG. 5B and 5C). To further confirm that the ECFC morphogenesis within the D-hydrogels is MMP dependent, MMP activities were inhibited using a broad-spectrum inhibitor of MMPs, GM6001 (Blatchley et al., 2019; Hanjaya-Putra et al., 2011). The tubulogenesis of ECFCs in D-hydrogels was inhibited in the presence of GM6001 (0.1 mM) when compared with the controls (FIG. 5D). It was found that the D-hydrogels do not significantly degrade along the culture period in the absence of cells (see FIG. 1E), and next examined was matrix stiffness following ECFC encapsulation. We found that the D-hydrogels are softer on day 3 of culture (FIG. 5E). When supplementing with MMP inhibitor, GM6001, in the culture media, day 3 hydrogels were found to be significantly stiffer compared with those without MMP inhibitor, further supporting the conclusion that the vascular bed formation in the D-hydrogels is cell-mediated matrix degradation.

As the deposition of ECM may contribute to the progression of vascular morphogenesis (Chen et al., 2019; Marchand et al., 2019) next examined were the expression of collagen IV (ColIV) and laminin, the two major components of the basement membrane deposited by ECs. A significant increase in ColIV and laminin expression was found in both D- and N-hydrogels along with the culture period (FIG. 5F). On day 1 following ECFC encapsulation, the expressions of ColIV and laminin in D-hydrogels were similar to their expression in the N-hydrogels, while significantly higher in D-hydrogels on day 3. Localization of ColIV in the basement membrane of vessels in D-hydrogel in addition to the formation of a large expended inner lumen, both of which could not be observed in the N-hydrogels (FIG. 5G and 11B).

These results indicate that the timeline of ECM deposition is longer than the observed phenotype differences occurring at the onset of vasculogenesis. These results further demonstrate that D-hydrogels promote better cell-material interactions, resulting in further remodeling of the matrix that is less prominent in the N-hydrogels.

Collectively, the findings above suggest that the D-hydrogel environment facilitates matrix remodeling and degradation, allowing the cells to invade their surroundings and build up vascular tissues.

### Activation of FAK Regulates Integrin Clustering, Cell Contractility, and the Subsequent Formation of the Vascular Bed

To further delineate the role of FAK signaling and its contribution to integrin clustering and vasculogenesis in D-hydrogels, an inhibition study was performed, in which FAK phosphorylation was inhibited using the FAK inhibitor FI 14 (Damayanti et al., 2017). ECFCs were embedded in D-hydrogels and subsequently treated with FI 14. The inhibitor-treated constructs showed a significant reduction in sprouting (FIG. 6A, 6B, 11C, and 11D). Furthermore, analysis of the vasculature revealed that the constructs treated with the inhibitor underwent a significant reduction in their ability to form a vascular bed, as shown by a decrease in tube length and volume (FIG. 6C and 6D). These findings demonstrate that FAK is not involved in the formation of vacuoles and lumen; however, its activity is key for the progression of vasculogenesis, promoting cell sprouting and subsequent building of vascular tubes and networks.

To determine the contribution of FAK activity to FA formation and stability, the inventors analyzed the expression levels of integrin 31 and integrin aV mRNA levels in inhibitor-treated cells. It was found that the inhibition of FAK significantly decreases integrin expression, thereby inhibiting the formation of FAs and the progression of vasculogenesis (FIG. 6E). To further analyze the contribution of FAK to integrin clustering, the inventors analyzed the levels and localization of pMLC in D-hydrogels treated with the FI for 24 h. It was found that FAK inhibition does not promote the localization of pMLC to the nucleus, as observed in the N-hydrogel. However, FAK inhibition subsequently reduces the phosphorylation of MLC and therefore negatively influences cell contractility and in-tegrin clustering (FIG. 6F).

To further address the function of FAK on cell sprouting and vasculogenesis, the inventors stained the inhibitor-treated ECFCs for MT1-MMP expression after 24 h in culture. ECFCs treated with FI 14 exhibit significantly lower expression at protein and mRNA levels (FIG. 6G and 6H). Further analysis of MMP expression showed a significant reduction in MMP1 and MMP9 mRNA levels in cells treated with FI 14, as compared to untreated controls (FIG. 6H).

These findings support the hypothesis that FAK is important in maintaining and mediating FA stability. In addition, FAK activation promotes integrin clustering via the promotion of MLC phosphorylation and mediation of integrin expression. Furthermore, FAK participates in integrin-mediated downstream signaling leading to the upregulation of various MMPs, thereby enabling the cell sprouting, branching, and cell migration necessary for vascular morphogenesis.

### In Vivo Vasculogenic and Angiogenic Effects of Dynamic Networks

To determine how dynamic networks affect vascular formation in vivo, the inventors examined both vasculogenesis and angiogenesis. For vasculogenesis, pre-prepared GFP-ECFCs-loaded D-hydrogels and N-hydrogels (100 mL for each) were directly implanted subcutaneously in immunodeficient mice (nu/nu mice). Confocal images of GFP-ECFCs in the extracted hydrogel constructs revealed that on both days 3 and 5, higher cell densities with more sprouting and branching cells are present in D-hydrogels compared with N-hydrogels, which is consistent with in vitro results (FIG. 7A). Low-magnification images of histological sections stained for CD31 show that the volumes of D-hydrogels are smaller than those of the N-hydrogels on days 3 and 5, suggesting that D-hydrogels degraded faster than N-hydrogels (FIG. 12A, 12Bi, and 12Bi′). High-magnification images of those histological sections show numerous CD31⁺ cells distributed throughout the D-hydrogel on both days 3 and 5. In contrast, there were fewer CD31⁺ cells present in the N-hydrogel constructs (FIG. 12A, 12Bii, and 12Bii′). Moreover, the CD31⁺ endothelial lining of micro-vessels can be observed in both D-hydrogel and N-hydrogel constructs (FIG. 12A, 12Biii, and 12Biii′). Quantification of the density of these CD31⁺ micro-vessels revealed more vessels in the D-hydrogels compared with the N-hydrogels (FIG. 12C). On day 7 of transplantation, longer sprouts and tubes could be observed (FIG. 7A). To further confirm the pathways we identified in vitro, the inventors analyzed pFAK expression in implanted ECFC-loaded D- and N-hydrogels. Significant increases in FAK activation was found as indicated by the overall intensity of pFAK (FIG. 7B). Furthermore, the inventors observed enhanced 31-integrin clustering in D-hydrogels in vivo compared to N-hydrogels (FIG. 7C). The quantification of pFAK intensity and β1-integrin cluster size confirmed significant increases in D-hydrogels (FIG. 7D).

Next examined was how hydrogel network dynamics modulate host angiogenesis into the hydrogelin immunocompetent mice (C57BL/6 mice). For this, the inventors transplanted acellular hydrogels with stromal cell-derived factor-1a (SDF-1a). The inventors chose to use acellular gels to be able to isolate the impact of hydrogel network dynamics on angiogenesis. As SDF-1a is secreted following injury to recruit vasculature (Petit et al., 2007; Yamaguchi et al., 2003), the inventors sought to mimic this and examine whether the hydrogel properties, independently of the chemotactic signaling, modulate angiogenic response from the host. After 3 days, the inventors could observe cellular infiltration into the D-hydrogel with perfused vasculature at the edge of the hydrogel, while few cells could be observed penetrating the N-hydrogel (FIG. 7E and 13A). To further demonstrate the functionality of the infiltrating vessels, Evans blue dye was injected intravenously before the explanation of acellular gels on day 3. In situ confocal imaging shows perfused vessels, stained with lectin, within the D-hydrogel (FIG. 7C, 13B, and 13C). Overall, these results suggest that the dynamic networks facilitate rapid vasculogenesis in vivo, as well as host angiogenesis and circulation, further extending the observation to applications of vascular regeneration.

### Defining the Mechanism for Fast Vasculogenesis Driven by D-Hydrogel Networks

Studies have begun to explore the role of the dynamics of hydrogels during cell differentiation and migration. However, assembly of a tissue requires a coordinated sequence of events to occur in a temporal and spatial manner in neighboring cells. Specifically, in vascular morphogenesis, endothelial cells undergo vacuolization followed by synchronized sprouting and branching events, forming a complex bed of tubular structures (Bayless et al., 2000; Crosby and Zoldan, 2019; Davis and Bayless, 2003; Davis and Camarillo, 1996; Davis and Senger, 2005; Iruela-Arispe and Davis, 2009). This process initiates with rapid matrix-integrin binding and proceeds with matrix degradation. To identify and understand the role of stress relaxation in this process, a tailored hydrogel system was required that not only decouples hydrogel network dynamics from other physical properties, mainly stiffness, but also allows the process of tissue assembly to progress. Using the disclosed gelatin/dextran-based D-hydrogel and N-hydrogel systems, shows that the stress relaxation of D-hydrogel networks, independent of stiffness, promote the interaction of cell surface integrin with the RGD sites of the matrix. The initial integrin-RGD interactions then lead to the recruitment of FAK and other adaptor proteins such as talin (del Rio et al., 2009; Tado-koro et al., 2003) and paxillin (Brown et al., 1996; Mofrad et al., 2004) in a non-stiffness and mechanosensitive manner. Following this first step of FA formation, pMLC localization to the actin cytoskeleton and actin contraction then leads to the aggregation of distant integrin clusters and the formation of larger FAs with the recruitment of vinculin and further FAK activation. Activated FAK then further contributes to the phosphorylation of MLC and integrin expression, therefore supporting the formation of larger integrin clusters. Thus, FAK is a key mediator in the integrin clustering, stable FA formation, and downstream signaling necessary for cell migration and vessel formation.

FAK-mediated downstream signaling leads to an increase in MT1-MMP, MMP9, and MMP1 expression, which leads to the degradation and subsequent remodeling of the matrix, allowing the cells to sprout, branch, and ultimately form an expansive vascular bed (FIG. 7G). Matrix network dynamics is hereby a key mediator of vascular tissue assembly. A non-dynamic matrix prevents contraction-mediated integrin clustering, leading to the abrogation of cell signaling, which ultimately leads to the inhibition of vascular morphogenesis.

Vascularization is a key process that enables the engineering of tissues, either healthy or cancerous. The implications of these results serve not only toward our understanding of how hydrogel network dynamics influence vascular morphogenesis but also as a framework for designing hydrogel biomaterials that can be applied to studying complex tissue assembly and morphogenesis and toward a range of therapeutics.

### Investigation of Stress Relaxation Resulting from Sarcoma Cells

### ECM Composition Impacts Bulk Tumor Stress Relaxation Time Independent of Stiffness

This study aimed to characterize biologically relevant ranges of stress relaxation time and focused on assessing the impact of ECM composition on tumor stress relaxation time. As a result, the inventors assessed tumor bulk stress relaxation time of soft tissue sarcoma using immunohistochemistry (IHC) and rheology (FIG. 14A). To minimize the impact of tumor heterogeneity, the inventors used a mUPS cell line with specific biological consequences. mUPS Collagen VI knockdown (ColVI KD) and scrambled (Scr) cell lines have been developed and validated by the University of Pennsylvania. The inventors validated that knockdown was stable using IHC (FIG. 14B). Due to the large tumor volume variability recorded on Day 10, the inventors used tumor volume to size-match tumors, aiming for volumes below 250 mm³ to avoid heterogeneity and hypoxic core formation (FIG. 14C). mUPS ColVIKD and Scr tumors did not differ appreciably in mass on day of analysis (FIG. 14D). Stress relaxation curves indicate that mUPS ColVIKD tumors relaxed significantly slower than mUPS Scr counterparts (FIG. 14E). mUPS ColVIKD stress relaxation quarter-time (T_{1/4}) was over one order of magnitude greater than mUPS Scr T_{1/4} (FIG. 14F). While it was expected that mUPS ColVIKD would be markedly less stiff than the mUPS Scr; there was no statistically significant difference in stiffness (FIG. 14G). Thus, it was evident that specific changes in ECM composition alter bulk tumor stress relaxation time without impacting stiffness.

### Quick- and Slow-Relaxing Hydrogels Exhibit Relaxation Times Differing by One Order of Magnitude

To study the impact of stress relaxation time on cellular behavior, the inventors used a controlled hydrogel model. Quick- and slow-relaxing hydrogels were composed of two biopolymers commonly used for tissue engineering, gelatin and dextran utilizing dynamic covalent bonds, made by imine and arylhydrazone, to synthesize quick-relaxing hydrogels and static covalent bonds, made by methacrylates, to make slow-relaxing hydrogels (FIG. 15, FIG. 16). The quick-relaxing hydrogels were composed of adipic acid dihydrazide-modified gelatin (Gel-ADH) and oxidized dextran (Dex-CHO) while the slow-relaxing hydrogels were composed of methacrylate-modified gelatin (Gel-MA) and glycidyl methacrylate modified dextran (Dex-MA) (FIG. 15A). The quick-and slow-relaxing hydrogels were fabricated to have similar initial elastic moduli ˜175-200 Pa (FIG. 15B). The relaxation rate was then quantified as the time of the initial stress to decrease to half of its original value (T_{1/2}) to confirm that the slow-relaxing hydrogels relaxed one order of magnitude slower than the quick-relaxing hydrogels (FIG. 15C). The T_{1/2} of quick-relaxing gels was ˜10³ seconds, while the T_{1/2} of slow-relaxing gels was ˜10⁴ seconds.

### Individual Cells Encapsulated in Quick-Relaxing Hydrogels Interact with the Matrix Through Filopodia-Like Protrusions

Upon establishing the model, the inventors performed single-cell encapsulations of sarcoma cells in quick- and slow-relaxing hydrogels (FIG. 17A). The inventors encapsulated mUPS dispersed cells in quick-and slow-relaxing hydrogels and observed their interactions with the matrix from post-encapsulation (PE) Day 0 to PE Day 3 (FIG. 17B). It was expected for encapsulated mUPS-dispersed cells to be highly migratory in the quick-relaxing hydrogels and to observe their migration on Day 2; however, there was no significant evidence of the mUPS-dispersed cells migrating in the hydrogels (data not shown). However, it was observed that mUPS cells in quick-relaxing hydrogels begin to form protrusions on PE Day 2 and form larger filopodia and lamellipodia on PE Day 3. mUPS cells in slow-relaxing hydrogels did not form protrusions through PE Day 3. The inventors focused on assessing the structure of the initial cell-matrix interactions and performed IF on mUPS cells encapsulated in the hydrogels on PE Day 1. To examine the formation of FAs and possible differences in generated traction forces, the inventors performed IF for f-actin and phosphorylated myosin light chain (pMLC) (FIG. 17C). The inventors observed the presence of f-actin+filopodia-like protrusions in the quick-relaxing hydrogels, and no such protrusions were present in the slow-relaxing hydrogels. The presence of f-actin+filopodia-like protrusions confirms the recent two-dimensional work by Adebowale et al. showing that HT1080 cells migrating on soft, quick-relaxing substrates for filopodia-like protrusions ²³. However, when total f-actin clusters were quantified, no difference was found in the number of clusters for mUPS cells encapsulated in quick-and slow-relaxing hydrogels (FIG. 17Di). There was also no observed quantitative differences in pMLC signal between the two conditions (FIG. 17Dii).

### Sarcoma Spheroids Encapsulated in Quick-Relaxing Hydrogels Grow; Those in Slow-Relaxing Hydrogels Shrink

The inventors aimed to address how host ECM stress relaxation impacts early stages of primary tumor growth (FIG. 18A). Thus, the inventors encapsulated murine UPS (mUPS) spheroids in quick- and slow-relaxing hydrogels and followed their growth PE using light microscopy (FIG. 18B). As expected, mUPS spheroids encapsulated in quick-relaxing hydrogels grew from PE 0 to PE 3 (FIG. 18B). Interestingly, mUPS spheroids encapsulated in slow-relaxing hydrogels from PE 0 to PE 3 not only failed to grow but also shrank appreciably from PE 0 to PE 3 (FIG. 18B). The most dramatic single-day decrease in normalized spheroid area was from PE Day 0 to PE Day 1. First, the inventors wanted to determine that the results were not due to cell apoptosis. The inventors performed TUNEL staining on mUPS spheroids encapsulated in the two hydrogels (FIG. 19), and did not find any significant differences in apoptosis nor the presence of an apoptotic core in mUPS spheroids encapsulated in slow-relaxing hydrogels. Then, it was verified whether the differences seen were entirely contractility mediated. This was accomplished with co-encapsulated mUPS spheroids in quick-relaxing hydrogels with blebbistatin (FIG. 18C). It was found that while blebbistatin contractility inhibition recapitulated some of the differences between quick- and slow-relaxing hydrogels, it was not sufficient to fully mirror spheroid shrinkage in slow-relaxing hydrogels. F-actin expression was examined to assess mUPS cellular morphology in the hydrogel systems. It was found that mUPS spheroids encapsulated in slow-relaxing hydrogels exhibit a paucity of polymerized actin (FIG. 18D).

### Sarcoma Spheroids Encapsulated in Quick-Relaxing Hydrogels Exhibit Increased Number of Focal Adhesions

To examine the pathways responsible for differences in spheroid growth in quick- and slow-relaxing hydrogels and to address the implication on FA formation, the inventors performed IF staining and imaging of hydrogel-encapsulated mUPS spheroids. Since it was previously recorded that the most dramatic size difference was seen between PE Day 0 and Day 1, this study focused the analysis on PE Day 1 spheroids. First, the study further examined FA formation and confirmed that, consistent with prior studies in this hydrogel model, mUPS spheroids in quick-relaxing hydrogels have increased incidence of integrin clustering (Wei et al., 2020). The inventors utilized IF to evaluate integrin β1 expression and found increased integrin β1 clusters in the mUPS spheroid cores of quick-relaxing gels (FIG. 20A). Then, the study examined IF for vinculin expression to assess the presence of mature FAs (FIG. 20B). mUPS spheroids encapsulated in quick-relaxing hydrogels had a greater number of Vinculin+ cells in both the spheroid core and edge.

### PLOD2 is Not Responsible for Spheroid Growth in Quick-Relaxing Hydrogels

Recent studies have identified that PLOD2 is a key regulator of integrin β1 and that PLOD2-mediated hydroxylation stabilizes integrin β1 in squamous cell carcinoma (Ueki et al., 2020). Prior studies have confirmed that quicker stress relaxation times increase mUPS PLOD2 expression, therefore it was examined whether PLOD2 was responsible for FA formation and increase in quick-relaxing hydrogels (Lewis e al., 2019). First, the inventors performed IF for PLOD2 and found no difference in PLOD2 expression between quick- and slow-relaxing hydrogels in either the mUPS spheroid core or edge (FIG. 21A). Light microscopy was also used to image an mUPS PLOD2 knockdown (PLOD2⁻) and scrambled (Scr) control spheroids to examine whether absence of PLOD2 would be sufficient to reduce spheroid growth or induce spheroid shrinkage in quick-relaxing hydrogels (FIG. 21B). No statistical differences were found between mUPS PLOD2⁻ and mUPS Scr spheroids grown in quick-relaxing hydrogels from PE Day 0 to PE Day 2.

### Sarcoma-Associated Angiogenesis and Intravasation in Quick Relaxing Hydrogels

Cancer-mediated angiogenesis and vascular intravasation are key early steps of the metastatic cascade. Studies from have confirmed that quick-relaxing hydrogels increase angiogenesis (Wei et al., 2020). This study aimed to use the hydrogel model to evaluate the impact of local changes in the ECM to cancer-associated angiogenesis using human endothelial colony-forming cells (hECFCs) and, to match species, human fibrosarcoma cells (HT 1080s). First, the inventors confirmed that HT 1080 spheroids follow the same trends as mUPS cells in quick- and slow-relaxing hydrogels (FIG. 22). Then, the inventors co-encapsulated HT1080 spheroids with hECFCs and tracked vascular network formation over 3 (5?) days.

### Discussion

This study examines the impact of stress relaxation on sarcoma spheroid growth and single-cell matrix interactions. Specifically, the study explores whether quicker stress relaxation times change sarcoma-matrix interactions. The inventors moved to a disperse single-cell encapsulated model to further probe the mechanism. It was found that mUPS-dispersed cells encapsulated in quick-relaxing gels exhibited filopodia-like protrusions while those encapsuled in slow-relaxing gels did not. This result was consistent with published results finding HT1080 cells on two-dimensional soft, quick-relaxing substrates exhibit more filopodia at the leading edge and small nascent adhesions (Adebowale et al., 2021). Additionally, the findings confirm those of Adebowale et al. in a three-dimensional system and suggest that early-stage cancer tumors may use this mode of migration in quick-relaxing host tissue. It was originally hypothesized that, consistent with dispersed single cells in quick-relaxation environments, sarcoma spheroids in quick-relaxation environments would grow and cells would migrate outward. It was found that mUPS spheroids in quick-relaxing hydrogels grow but, surprisingly, the same spheroids in slow-relaxing hydrogels shrink. This behavior was also found to be consistent for HT1080 cells. mUPS spheroids encapsulated in quick-relaxing hydrogels displayed mature f-actin fibers, while those in slow-relaxing hydrogels exhibited a scarcity of polymerized f-actin, suggesting limited ability to deform the surrounding matrix. It was found that cells in mUPS spheroids encapsulated in quick-relaxing hydrogels had more vinculin expression and greater numbers of integrin β1 clusters, suggesting that they formed more FAs. It was examined whether PLOD2 was responsible for mediating integrin β1 expression and found that the phenomenon was PLOD2-independent.

This study utilized a hydrogel model with tunable stress relaxation properties to address the contribution of this mechanical property to early-stage tumor growth and migration. From these studies, in a dispersed cell encapsulation model, sarcoma cells in quick-relaxing hydrogels formed filopodia despite maintaining a rounded morphology, a mode of cell migration only previously reported in on two-dimension substrates. Also observed was increased sarcoma-matrix interactions, in the form of FAs and filopodia, in the quick-relaxing hydrogels. It was deduced that quick-relaxing hydrogels lead to increased spheroid growth via the formation of mature FA's. Slow-relaxing hydrogels led to spheroid shrinkage, f-actin deficiencies, and fewer FA's.

### EXAMPLES

### GFP-ECFCs Cell Lines

Primary human endothelial colony forming cells (ECFCs) were a gift from M. Yoder (Indiana University) and GFP-expressing primary human ECFCs (GFP-ECFCs) were generated and provided by K. Eisinger (University of Pennsylvania), following previously described protocol (Eisinger-Mathason et al., 2013)). Cells were maintained as previously described (Blatchley et al., 2019). In brief, cells were cultured on collagen type I (BD Biosciences) coated dishes in endothelial growth media (EGM 2, Lonza) media containing 10% FBS (Hyclone) and VEGF (R&D Systems) with media changes every other day. When cells reached confluency they were incubated with 0.5% Trypsin/EDTA solution (ThermoFisher Scientific) until fully detached. Cells were collected in culture medium, centrifuged and either embedded into hydrogels for further experiments or expanded on collagen type I culture flasks. For all experiments cells were used between passages 6-9.

### In Vivo Studies

The 7-8 weeks old female nude mice (Charles River) and 6-8 week old C57BL/6 male mice (The Jackson Laboratory) were used for the in vivo studies. Mice were kept under specific pathogen-free conditions in the medical school of Johns Hopkins University, Division of Animal Resources. They were housed with a maximum of 5 mice per cage prior to the experiments. Mice were randomly grouped for subsequent subcutaneous experiments. All animal procedures complied with the NIH Guidelines for the Care and Use of Laboratory Animals and were approved by the Institutional Animal Care and Use Committee.

### Synthesis of Adipic Acid Dihydrazide Modified Gelatin (Gtn-ADH)

Gtn-ADH was synthesized via a previously described protocol (Hozumi et al., 2018) that was further modified.

Gelatin (Type A, Sigma-Aldrich; 1.0 g) was dissolved in a phosphate buffered saline (PBS) (40 mL, pH 5.5), followed by the addition of adipic acid dihydrazide (ADH; >98%; Sigma-Aldrich) (1.74 g, 10 mmol) at 50° C. or 55° C. with stirring. Next, 1-hydroxybenzotriazole hydrate (HOBt; >97%) (0.77 g, 5.7 mmol) was dissolved in DMSO (5 mL) and added dropwise to the mixture. Then, N-(3-dimethylaminopropyl)-N′-ethylcarbodiimide hydrochloride (EDC; crystalline; Sigma-Aldrich) (0.77 g, 4.0 mmol) was directly added to the mixture and the pH was adjusted to 5.0-5.3 with NaOH (1M) and/or HCl (1M). The mixture was stirred overnight at 50° C. or 55° C. and the solution became bright canary yellow. The pH was re-confirmed and adjusted to 5.1 by NaOH (1M) and HCl (1M). If the pH was outside the bounds of 5.0-5.3 the following day, the batch was discarded. The product was purified by dialysis (MWCO 8000) against distilled water (DW) or reverses osmosis waster (RO) for a week with the water changed twice every day, followed by lyophilization to obtain Gtn-ADH.

New proton peaks at 1.7 ppm and 2.5 ppm indicated the coupling of ADH on the gelatin chains from the ¹H NMR spectra (FIG. 1H), which was recorded in D₂O on a 400 MHz spectrometer (Bruker Avance). The degree of modification was evaluated by trinitrobenzene sulfonic acid assay (TNBS, 5.0% w/v) described previously (Freedman and Radda, 1968). This assay indicated that 32.7% of the carboxy groups were substituted with hydrazide groups under the reacted conditions in this study.

### Synthesis of Multi-Aldehyde Modified Dextran (Dex-CHO)

Dex-CHO was synthesized via a previously described protocol (Wei and Gerecht, 2018) that was further modified.

Dextran (Mn=110,000, Sigma-Aldrich; 1.0 g, 6.2 mmol) was dissolved in DW (100 mL), and then sodium periodate (>99.0%, Sigma-Aldrich; 1.0 g, 4.6 mmol) in distilled water (1 mL) was added dropwise. The solution was stirred in the dark, using aluminum foil, for 20 or 30 min. The oxidation reaction was terminated by adding ethylene glycol (1.0 mL) and stirring for an additional 30 min. The mixture was then dialyzed (MWCO 3500) against DW or RO water for a week with the water changed twice every day, followed by lyophilization to obtain Dex-CHO.

The oxidation percentage of Dex-CHO was about 22.8%, and determined by quantifying the number of aldehyde groups in the polymer using tert-butylcarbazate (t-BC, >98.0%, Sigma-Aldrich) as described previously (Maia et al., 2005). The aldehyde groups of Dex-CHO reacted with carbazates of t-BC (excess amount) to form carbazones, and the unreacted t-BC was quantified by adding trinitrobenzene sulfonic acid (TNBS, 5.0 wt. % w/v, Sigma Aldrich). The resulting colored trinitrophenyl-derivative was measured at 334 nm using a spectrophotometer (SpectraMax M3 Platereader).

### Synthesis of Methacrylate Modified Gelatin (Gtn-MA)

Gtn-MA was synthesized via a previously described protocol (Wei et al., 2016) that was further modified.

Gelatin (1.0 g) was dissolved in PBS (100 mL, pH 7.4) at 60° C., and methacrylic anhydride (94.0%, Sigma-Aldrich; 8.0 or 12.0 mL) was added into the solution, stirring for 3 hr or 8 hr at 50° C. The product was dialyzed at 40° C. (MWCO 8000) against DW or RO water for a week with the water changed twice every day, followed by lyophilization to obtain Gtn-MA.

The degree of methacrylation was determined as 78.1% from ¹H NMR spectra by integrating peaks at 7.3 ppm, and peaks at 5.4 ppm and 5.7 ppm, which corresponded to the aromatic residues of gelatin and methacrylamides, respectively (FIG. 1J). ¹H NMR spectra were recorded in D₂O on a 400 MHz spectrometer. A minimum degree of methacrylation of 70% was necessary for the hydrogel to crosslink.

### Synthesis of Glycidyl Methacrylate Modified Dextran (Dex-GMA)

Dex-GMA was synthesized via a previously described protocol (Liu et al., 2015).

Dextran (1.0 g, 6.2 mmol) was dissolved in DMSO (40 mL) and then 4-dimethylaminopyridine (DMAP, ≥99.0%, Sigma-Aldrich; 0.19 g, 1.55 mmol) and glycidyl methacrylate (GMA, 97.0%, Sigma-Aldrich; 0.44 g, 3.1 mmol) were added to the solution. The mixture was stirred at 50° C. overnight and an equimolar amount of HCl (1.55 mmol) was added to neutralize the DMAP. The product was purifier by dialysis (MWCO 8000) against DW for a week with the water changed twice every day, followed by lyophilization to obtain Dex-GMA.

The degree of substitution was calculated as 34.0% from ¹H NMR spectra by comparing the ratio of the areas under the proton peaks at 6.2 ppm and 5.3 ppm to the peak at 4.8 ppm (FIG. 1J). ¹H NMR spectra were recorded in D₂O on a 400 MHZ spectrometer.

### Synthesis of Dynamic Hydrogel (D-Hydrogel/Quick-Relaxing Hydrogel)

Gtn-ADH and Dex-CHO were dissolved into PBS (pH 7.4) at 60° C. for 24 hours, respectively. The solutions were mixed uniformly by pipette and then placed in 37° C. water bath or incubator. The transparent D-hydrogel was formed after several minutes. The total weight concentration of Gtn-ADH was kept as a constant of 5 wt % and the final weight concentration of Dex-CHO was kept as 0.5 wt % (stiff gel) or 0.25 wt % (soft gel).

### Synthesis of Non-Dynamic Hydrogel (N-Hydrogel/IQuick-Relaxing Hydrogel)

Gtn-MA, Dex-MA, and the photo-initiator 2-hydroxy-4′-(2-hydroxyethoxy)-2-methylpropiophenone (Irgacure 2959; Sigma-Aldrich) in PBS (pH 7.4) were mixed uniformly by pipette. The N-hydrogel was obtained by UV polymerization of 50 s (stiff gel) or 20 s (soft gel). The concentration of initiator 2959 was kept at a constant of 0.5 wt %. The weight concentrations of the Gtn-MA and Dex-MA were kept identical with the corresponding D-hydrogels.

In an alternative procedure, Gtn-MA and Dex-MA were dissolved in PBS (pH 7.4) at 60° C. for 24 hours. The photoinitiator, 2-hydroxy-4′-(2-hydroxyethoxy)-2-methylpropiophenone (Irgacure; Irgacure 2959; Sigma-Aldrich) was also dissolved in PBS (pH 7.4) at 60° C. for 30 minutes with vertexing every 5 minutes. The photoinitiator was filtered through a 0.22 μm filter prior to being mixed with slow-relaxing hydrogel precursors via pipetting. The concentration of Irgacure was kept at 0.5 wt %. The final weight concentration of Gtn-MA was kept at 5 wt % and the final weight concentration of Dex-MA was kept at 0.5 wt %. The slow-relaxing hydrogel was polymerized for 50 s under UV light.

### Rheological Measurements

The mechanical properties of the hydrogels were tested by a rheometer (AR-G2, TA instruments) equipped with 25 mm or 8 mm parallel plate at 37° C. For all rheology experiments, the hydrogels were first polymerized using sterile culture conditions in standard tissue-culture treated 96-well plates, placed in the incubator for 30 minutes, hydrated with PBS for an additional 30 minutes at 37° C., and then measured. The dynamic time sweeps were performed on the D-hydrogel samples in soft and stiff conditions respectively by using 25 mm plate. The G′ and G″ were monitored at a fixed strain of 0.1% and a fixed frequency of 1 Hz. The G′ of the well-formed D-hydrogels and N-hydrogels were also tested on this rheometer, equipped with 8 mm parallel plates at 37° C. All the D-hydrogels and N-hydrogels with various conditions were prepared as discs measuring 8 mm in diameter. At a fixed strain of 0.1%, the frequency sweep was performed on the samples and the data was collected from the platform region. A solvent trap wetted with PBS was used to prevent sample dehydration during the measurements. Moreover, the G′ of D-hydrogel and N-hydrogel encapsulated with ECFCs were also tested, at different time points along with the increased culture time, by the same method. In addition, stress relaxation measurements of both D-hydrogel and N-hydrogel were performed by time sweep tests at a constant initial strain of 10% and a fixed frequency of 1 Hz. The corresponding stress relaxation curves were normalized to their initial value and fitted to a stretched exponential function, σ/σ₀=e^{-(t/T)∧β} as previously reported (FIG. 9) (Brown et al., 2018), σ/σ₀ is the normalized stress, t is the experimental time, T is the time constant, and β(0<β<1) is the stretching exponent. The t is the experiment time and the fitting were performed in MATLAB. The half-time, T_{1/2}, was quantified as the time for the initial stress to decrease to half of its original value.

### Diffusion Tests of D-Hydrogels and N-Hydrogels

The softer D-hydrogels and N-hydrogels were immersed in the Rhodamine B (1.0 mg L⁻¹ in PBS; Sigma-Aldrich) at 37° C., then taken out at selected time points and inserted into the vials. 1 mL collagenase IV (0.05%) was added to each hydrogel for proteolytic degradation. After 30 min by which time point the hydrogels were fully degraded, the absorbance of each sample was measure using a microplate reader at a wavelength of 554 nm. D- and N-hydrogels in collagenase IV solution were set as the controls/blanks.

### Encapsulation of ECFCs in D-Hydrogels and N-Hydrogels

To encapsulate cells in D-hydrogel, the Gtn-ADH and Dex-CHO were dissolved into PBS (pH 7.4) respectively. The cell pellet was mixed with the Dex-CHO solution and VEGF (R&D Systems; 50 ng mL⁻¹) and bFGF (R&D Systems; 50 ng mL⁻¹) to obtain a cell suspension at a concentration of 4×10⁶ cell mL⁻¹. The Gtn-ADH solution was then added to this cell mixture and pipetted homogeneously. The mixture of 90 μL totally was transferred into a PDMS mold with 8 mm diameters and placed into the incubator for 30 min of gelation, before adding 1 mL of EGM-2 (Lonza) with 10% FBS (Hyclone), VEGF (R&D Systems; 50 ng mL⁻¹) and bFGF (R&D Systems; 50 ng mL⁻¹) (Stratman et al., 2011). The media was replaced every 24 hr. To encapsulate cells in N-hydrogel control, the cell pellet was mixed with the Gtn-MA, Dex-MA and Irgacure 2959 uniformly mixed by pipette with PBS (pH 7.4) with VEGF (R&D Systems; 50 ng mL⁻¹) and bFGF (R&D Systems; 50 ng mL⁻¹) to obtain a cell suspension at a concentration of 4×10⁶ cell mL⁻¹, which was then transferred into the PDMS mold with 8 mm diameters. The hydrogels were obtained by UV polymerization, then adding 1 mL of EGM-2 (Lonza) with 10% FBS (Hyclone) with VEGF (R&D Systems; 50 ng mL⁻¹) and bFGF (R&D Systems; 50 ng mL⁻¹) with replacement of every 24 hr. The morphologies of the encapsulated ECFCs were observed and tracked by optical microscopy (phase-contrast) and confocal microscopy (LSM 780, Zeiss).

### Cytocompatibility of D-Hydrogel and N-Hydrogel Components

The cytotoxicity was investigated by WST-1 assay (Roche) according to the manufacturer's instructions. In brief, 2×10⁴ cells were cultured in 100 mL EGM-2 media (Lonza) with 10% FBS (Hyclone) in each well of 96-well plate and treated by Gtn-MA (5 wt %), Gtn-ADH (5 wt %), Dex-MA (0.5 wt %), Dex-CHO (0.5 wt %), Irgacure 2959 (0.5 wt %) and 50 s UV, respectively. PBS only, Gtn and Dex were set as controls. After 24 hr incubation, 10 μL of WST-1 mixture was added to each well. After placing in an incubator for another 2 hr, the absorbance of each sample was measured using a microplate reader at a wavelength of 450 nm. Cell viability was determined as the percentage of PBS controls.

### Traction Force Microscopy

D-hydrogel and N-hydrogel precursor solutions were prepared as stated above. 1 μm FluoSpheres (ThermoFisher Scientific) that are carboxylated were rinsed with PBS 2 times and then mixed with the GFP-ECFC pellet prior to hydrogel formation. The inventors have chosen 1 μm beads as they are large enough to neglect Brownian displacements (Bloom et al., 2008). Using a confocal microscope (LSM 780, Zeiss) z stacks of beads were taken in 1 min intervals for 20 min to observe bead movement, according to a previously established method (Bloom et al., 2008).

### FAK-Specific Inhibition (FI 14), MMP Inhibition (GM6001), and Blebbistatin Inhibition Studies

For 2D screening of the FI 14 (FI-14, R 95.0%; Sigma-Aldrich), the ECFCs were seeded into a 96-well plate at a cell density of 10000 cells/well in EGM-2 media (Lonza) with 10% FBS (Hyclone). After 1 day of culture, 100 μL of EGM-2 media containing increased concentrations of FI 14 ranging from 0 to 10 μM were added per well and culture for additional 24 hr following by staining for phospho-FAK (Tyr397;ThermoFisher Scientific) as described below. Biological triplicates were tested for each FI 14 concentrations. For inhibition studies, ECFCs were suspended in the hydrogel precursor as above with the addition of 10 mM of FI-14 (Sigma-Aldrich) or GM6001 (1 mg mL⁻¹; Sigma-Aldrich), 60 mM Blebbistain (Sigma-Aldrich) or vehicle control (DMSO; Sigma-Aldrich). The corresponding culture media also supplied with the corresponding inhibitor/vehicle control at the same concentration. Media was replenish every 24 h till analysis.

### Vacuole Visualization and Quantification

Quantification of vacuoles and lumen formation in 4-8 hr was performed using light microscopy as previously reported (Bayless et al., 2000). For each D-hydrogel and N-hydrogel condition, the inventors analyzed biological triplicates with 2-3 images per replicate for vacuole and lumen formation. A cell was considered to be vacuolating if >30% of the cell's area contained a vacuole or lumen.

### Immunofluorescence (IF) of D-Hydrogel or N-Hydrogel

The D-hydrogel or N-hydrogel constructs were fixed with 2% paraformaldehyde (PFA; Sigma-Aldrich) for 20 min at room temperature, then washed three times with PBS with 10 min in between each wash. For staining, the encapsulated cells were permeabilized with a solution of 0.5% Triton-X (Sigma-Aldrich) for 20 min, followed by staining with primary antibody in antibody diluent solution (Life Technologies; 1:100) overnight at 4° C., then washed with PBS three times with 10 min in between each wash. Hydrogels were then incubated in a secondary antibody in antibody diluent solution (Life Technologies; 1:250) for 4 hours at room temperature and then washed with PBS three times with 10 min between each wash. Finally, the hydrogels were counterstained with DAPI (ThermoFisher Scientific; 1:1000) for 15 min at room temperature and then washed with PBS with 10 min in between each wash before analyzing using confocal microscopy. Primary antibodies of anti-Cdc42 (clone 11A1; Cell Signaling), anti-Integrin β1 (clone 4Br7; Santa Cruz Biotechnology, anti-Vinculin (Sigma-Aldrich), anti-phospho-FAK (Tyr397; ThermoFisher Scientific), anti-MMP14 (clone EP1264Y; abcam), anti-YAP (clone H-125; Santa Cruz Biotechnology), anti ColIV (abcam), anti-pMLC (Cell Signaling) were diluted in antibody diluent as 1:100, according to the manufacturer's protocol. Secondary antibodies of goat 488/546 (Invitrogen) or mouse 488/546 (Invitrogen) with 635 phalloidin (Invitrogen) were used at 1:250 in antibody diluent. Images were taken using fluorescence (AxioObserver Zeiss) or confocal (LSM 780 or LSM 800, Zeiss) microscopes. To image lumen, orthogonal views (z stacks) where taken and merged to the corresponding plain views (x,y, stacks) in the FIG. For IF-based quantification, the intensity of Cdc42, integrin β1 and p-FAK were normalized to the intensity of corresponding DAPI using ImageJ. Biological triplicates (10 cells per replicate, 30 cells total) in each condition were used for the analysis. The aspect ratio was also obtained by ImageJ from IF images of actin with biological triplicates (20 cells per replicate, 60 cells total). The analysis of FA and integrin cluster size, number and areas were performed by ImageJ as shown in previous publication (Humphries et al., 2007). Biological triplicates (10 cells per replicate, 30 cells totally) in each condition were used for this analysis. The quantification of vascular tube length and tube volume in D-hydrogel and N-hydrogel were analyzed using Imaris. Biological triplicates with 5-6 images per replicate were used for the analysis.

For nuclear protein quantification the images were analyzed using ImageJ software. The z stacks were analyzed using average intensity z-projections. DAPI staining was used to outline the nucleus and the nuclear pMLC signal intensity was normalized to overall pMLC intensity.

### Real-Time RT-PCR

Quantitative real time RT-PCR was performed as described previously (Hanjaya-Putra et al., 2011; Park and Gerecht, 2014). In brief, total RNA was isolated from ECFCs encapsulated in D-hydrogels or N-hydrogels using TRIzol according to the manufacturer's instructions. Two-step RT-PCR was performed using TaqMan Gene Expression Master Mix (ThermoFisher Scientific) according to the manufacturer's instructions: for MT1-MMP ((hs01037003_g1), Integrin β1 (hs00559595_m1), Integrin aV (hs00233808_m1), MMP1 (hs00233958_m1), MMP9 (hs00957562_m1), Collagen IV (hs00266237_m1) and laminin (hs00267056_m; all from ThermoFisher Scientific). All samples were examined in triplicate, analyzed, and graphed as previously described (Hanjaya-Putra et al., 2011; Park and Gerecht, 2014).

### In Vivo Subcutaneous Implantation of GFP-ECFC-Loaded D-Hydrogels and N-Hydrogels

To analyze in vivo vasculogenesis, the ECFCs or GFP-ECFC-loaded D- and N-hydrogels (pre-prepared as detailed above) were subcutaneously implanted into nude mice (7-8 week-old females; N=3) as in previous studies (Ghajar et al., 2006; Wei and Gerecht, 2018). To analyze vascularization of acellular hydrogel constructs (i.e. angiogenesis), gels were subcutaneously implanted in C57BL/6J mice (6-8 week-old males; N=3 per group). In both cases, the constructs were subcutaneously implanted and sutured into both flanks of the mice (100 ml of each side). To assess perfused vessels in the gel, Evans blue dye (Sigma-Aldrich) at 30 mg/kg; 150 mL in PBS was injected into the lateral tail vein prior to euthanasia. At each time point indicated, mice were euthanized and the constructs were removed with surrounding tissue, fixed in 3.7% PFA (Sigma-Aldrich). The explants were immediately analyzed using in situ confocal imaging and then proceeded to histological analysis. The animal studies were performed using a protocol #MO19E328 approved by The Johns Hopkins University Institutional Animal Care and Use Committee.

### Immunohistochemistry

For immunohistochemical analysis, paraffin embedded tissue sections (5 mm) were dehydrated through graded ethanol followed by heat mediated antigen retrieval and incubation of anti-CD31 (abcam; 1:500), anti-phospho-FAK (ThermoFisher Scientific; 1:200), and anti-integrin-β1 (Santa Cruz Biotechnology; 1:200) diluted in antibody diluent overnight at 4° C. For CD31 staining the ImmPRESS HRP anti-rabbit IgG polymer detection kit (Vector Laboratories) and DAB was used for detection followed by counterstaining with hematoxylin. Quantification was performed by ImageJ to count CD31⁺ positive cells and microvessels. For immunofluorescent detection of phospho-FAK and Integrin-β1 anti-rabbit IgG secondary antibody Alexa Fluor 546 conjugate (Invitrogen; 1:500) and anti-mouse IgG secondary antibody Alexa Fluor 546 conjugate (Invitrogen;1:500) were used. Slides were counterstained with DAPI (ThermoFisher Scientific; 1:1000). Quantification of overall expression was performed using ImageJ by calculating the integrated density of the signal for each cell.

### Quantification and Statistical Analysis

Characterization of hydrogels, including stiffness, stress relaxation, cytocompatibility, and diffusion rates, were performed in n=3 (with biological replicates) with technical duplicates; image based quantifications were performed in at least biological triplicates in technical duplicates and detailed throughout the methods and FIG. legends; qRT-PCR was performed in biological triplicates with technical triplicate. Analysis of stress relaxation of the hydrogel was performed using a custom MATLAB script. Analysis of cell aspects, fluorescence intensity and FA measurements were performed using ImageJ. The filaments of vascular networks in the hydrogels were quantified using Imaris. The statistical analysis was performed by GraphPad Prism 6. We also used this software to perform t tests to determine significance. All graphical data are reported as means+SD. Significance levels were set at *p<0.05, **p<0.01, ***p<0.001, and ****p<0.0001. All graphical data were reported.

### Cell Culture

mUPS was derived from a murine model of sarcoma, LSL-Kras^{G12D/+}, Ink4a/Arf^{fl/fl} as previously published (Lewis et al., 2019; Lewis et al., 2018; Eisinger et al., 2013). mUPS cells were authenticated previously (Lewis et al., 2019; Eisinger et al., 2013). mUPS scrambled control line (mUPS Scr) and PLOD2 knockdown line (PLOD2⁻) were generated previously (Lewis et al., 2019; Eisinger et al., 2013). Cells were grown in UPS media, which consisted of high-glucose DMEM with 10% FPS and 1% penicillin/streptomycin and passaged with 0.25% Trypsin-EDTA. All cells were used before passage 20 and were tested for Mycoplasma every quarter.

### Spheroid Formation

Spheroids were formed using hanging droplet culture. Briefly, cell monolayers were grown until ˜75% confluent, rinsed with PBS, and passaged with 0.25% Trypsin-EDTA. Hanging droplets of 10,000 cells/20 μL droplet were formed on the inside of the cover of a 60 mm pipette dish. 10 mL of PBS were placed in the bottom of the dish to act as a hydration chamber. Spheroids were cultured as hanging droplets for three days prior to encapsulation in hydrogels. 10 μl of media was added to each droplet on the second day of droplet culture.

### Encapsulation of Cells in Quick- and Slow-Relaxing Hydrogels

For single-cell encapsulation, we followed many previously published protocols in this discipline (Lewis et al., 2019; Wei et al., 2020; Lewis et al., 2018). We first prepared cell pellets of mUPS cells (7.5×10⁵ cells) in a 1.5 mL Eppendorf tube. We then mixed the pellet with hydrogel solution by gentle pipetting to give a homogenous cell suspension. After mixing, the solution was pipetted into the 96-well plate (BD Bioscience) used for hydrogel experiments.

For spheroid encapsulation, a similar protocol was followed with the adjustments for spheroids. We first collected 12 spheroids in a 1.5 mL Eppendorf tube, centrifuged for 3 minutes, and then pipetted off the media. All subsequent steps were identical to single-cell encapsulation. Cells in hydrogels were cultured under standard conditions and daily media changes of 200 μL.

### Normalized Spheroid Area Quantification

Light microscopy images of encapsulated cells were recorded daily at 4×, 10×, and 20× magnifications starting at post-encapsulation (PE) Day 0. For spheroid area quantification, 10× images were grouped by individual spheroid and the area of the spheroid was manually traced in FIJI (NIH) for each day. Days 1-3 were normalized to the size of the spheroid on Day 0.

### Viability and Cytocompatibility of Quick- and Slow-Relaxing Hydrogel Components

Cytotoxicity was measured via WST-1 assay (Roche) according to the manufacturer's instructions. Briefly, 20,000 cells were cultured in 100 mL of DMEM media (Corning) with 10% FBS (HI-FBS) in each well of a 96-well plate and treated with Gtn-MA (5 wt %), Gtn-ADH (5 wt %), Dex-MA (0.5 wt %), Dex-CHO (0.5 wt %), Irgacure (0.5 wt %) and 50 s UV. PBS only, Gtn, and Dex were set as respective controls. After 24 hours of incubation, 10 uL of WST-1 mixture was added to each well and the plate was placed in the incubator for 2 hours. Then the absorbance of each sample was measured using a microplate reader at a wavelength of 450 nm. Cell viability was determined with respect to PBS controls.

For live/dead assays, the Live/Dead Viability/Cytotoxicity Kit (Thermo Fischer Scientific, L3444) was used. Briefly, 20,000 cells were cultured in 100 ml of DMEM media (Corning) with 10% FBS (HI-FBS) in each well of a 96-well plate and treated with Gtn-MA (5 wt %), Gtn-ADH (5 wt %), Dex-MA (0.5 wt %), Dex-CHO (0.5 wt %), Irgacure (0.5 wt %) and 50 s UV. PBS only, Gtn, and Dex were set as respective controls. 2 mL of DMEM media with 8×10⁻⁶ M Calcein Am and 2×10⁻⁶ M ethidium homodimer-1 was added to the cells and incubated for 30 minutes in the dark at room temperature. The cells were washed twice with DPBS and imaged using a Zeiss (AxioObserver) epifluorecent microscope equipped with a controlled incubator (37° C. and 5% CO₂).

### Immunofluorescence (IF) of Hydrogels with Spheroids and Single-Cell Suspensions Encapsulated in Hydrogels

Depending on the sample, a different protocol for immunofluorescence (IF) was used. The 2D immunofluorescence protocol was used for hydrogels with spheroids, the 3D immunofluorescence protocol was used for single-cell suspensions encapsulated in hydrogels.

### 2D Immunofluorescence Sample Preparation and Staining

### Gelatin-Coating of Slides for Sectioning

500 mL of RO water was heated to 60° C., 2.5 g of gelatin (type A, 220 Bloom) was added and dissolved completely using a magnetic stirrer. 0.25 g of chromium potassium sulfate was added and dissolved completely using a magnetic stirrer. The solution was then filtered through a 0.22 um filter. Histology slides were placed on racks and dipped into the solution 5 times for ˜5 seconds each. The racks were removed and the slides were allowed to dry at room temperature for 48 hours. Slides were then stored at −20° C.

### Sample Preparation for Immunofluorescence

The quick- or slow-relaxing hydrogel constructs were fixed with 4% paraformaldehyde (PFA; Sigma-Aldrich) for 30 minutes at room temperature, then washed three times with PBS (pH 7.4) for 10 minutes between each wash. The gels were then placed in 15% sucrose a minimum of 24 hours or until the hydrogels sank. This was repeated with 30% sucrose, 50/50 volume 30% sucrose/OCT compound solution (Tissue-Tek), and 100% OCT. The hydrogels were then embedded in OCT, frozen, and sectioned to 10 μm thickness using a cryostat (Leica CCM1950). Gelatin-coated, +charged slides were used for sectioning. The sectioned samples were then used for immunofluorescence staining.

### 2-D Immunofluorescence Staining

The sectioned samples were fixed in 4% paraformaldehyde for 15 minutes, washed 3 times in DPBS for 5 minutes, permeabilized in 0.5% Triton X-100 (Sigma-Aldrich) in DPBS for 10 minutes, stained with DAPI (Roche Diagnostics) for 5 minutes, washed with PBS 1 time for 5 minutes, and then screened for spheroids. At this point, any slides containing no sections with spheroids were discarded. The remainder were permeabilized with 0.05% TritonX-100 for 10 minutes, blocked in 3% GSA for 1 hour, and incubated overnight at 4° C. with the primary antibody in antibody diluent (DAKO). After washing 3 times for 5 minutes with PBS, they were incubated for 1 hour with the secondary antibody in antibody diluent (DAKO) at room temperature, washed 3 times for 5 minutes with PBS, stained with DAPI(Roche Diagnostics) for 2 minutes, washed 2 times for 5 minutes in PBS, and mounted using fluorescent mounting media (DAKO) and dried for 24 hours at 4° C. For samples that were stained with phalloidin, the primary antibody step and the washing directly following was skipped. Primary antibodies used included: anti-Vinculin (Sigma-Aldrich V9131), anti-PLOD2 (Proteintech 21214), anti-Integrin β1 (Santa Cruz sc-9970), anti-phosphorylated Myosin Light Chain (Cell Signaling 3674). Phalloidin used was: Alexa Fluor 488 Phalloidin, Alexa Fluor 635 Phalloidin. Secondary antibodies included: Goat anti-Rabbit Secondary Antibody, Alexa Fluor 488, Donkey anti-Mouse Secondary Antibody, Alexa Fluor 546 and various other combinations. Images were acquired using a Zeiss LSM 780 confocal microscope.

### Apoptosis Staining

For apoptosis detection, the TdT Apoptosis Detection Kit—Fluorescein (Biotechne, 4812-30-K) was used. Briefly, cryosectioned hydrogels with spheroids were dried overnight at room temperature. Then, samples were rehydrated by immersing for 5 minutes each in 100%, 95%, and 70% ethanol in sequence. Samples were then washed once in PBS for 5 minutes, fixed with 4% paraformaldehyde for 10 minutes at room temperature, washed once in PBS for 5 minutes. Then, samples were stained with Hoechst (Thermo Fischer 33342) for 5 minutes, washed with PBS one time for 5 minutes, and then screened for spheroids. Slides with hydrogels with spheroids were selected to continue the staining process. Said slides were washed once in PBS for 10 minutes at room temperature, covered with 50 μL of Proteinase K solution (1:50 dilution) and incubated for 30 minutes at Room temperature, washed twice for two minutes in RO water. Then, slides were immersed in 1× TdT labeling buffer for 5 minutes, covered with 50 μL of labeling reaction mixture (Mn²⁺) and incubated in a cell culture incubator (37° C. and 5% CO₂) for one hour. Samples were then immersed in 1× TdT stop buffer for 5 minutes at room temperature, washed twice for 5 minutes in PBS, covered with 50 μL of Strep-Fluorescein solution and incubated for 20 minutes at room temperature in the dark. Finally, samples were washed twice for two minutes in PBS. Samples were then mounted using fluorescent mounting media (DAKO) and dried for 24 hours at 4° C. Images were acquired using a Zeiss LSM 780 confocal microscope.

### 3-D Immunofluorescence Sample Preparation and Staining

The quick- or slow-relaxing hydrogel constructs were fixed on ice with methanol, pre-chilled to 4° C., for 5 minutes, then washed twice with DPBS, pre-chilled to 4° C., for 5 minutes. Hydrogels were then washed one time in room-temperature DPBS, for 5 minutes.

Hydrogels were then permeabilized with 1% Triton-X-100 (Sigma-Aldrich) in DPBS for 15 minutes, washed twice with DPBS for 5 minutes, blocked in 10% BSA for 1 hour, washed once with 0.05% Tween (in DPBS) for 5 minutes, and incubated with primary antibody in antibody diluent (DAKO) overnight at 4° C. On the second day, hydrogels were washed thrice with 0.05% Tween (in DPBS) for 10 minutes, incubated with secondary antibody in antibody diluent (DAKO) for 2 hours, washed thrice with 0.05% Tween (in DPBS) for 10 minutes, stained with DAPI (Roche Diagnostics) for 10 minutes, washed with PBS thrice for 5 minutes, and imaged. Primary antibodies, secondary antibodies, and phalloidin used are detailed above. Then, a short clearing protocol was performed, as published previously³³. Briefly, 330 ml of glycerol, 70 ml of RO water, and 297.2 grams of fructose were mixed into a uniform solution. Hydrogels were incubated in said solution overnight at 4° C. to allow for hydrogel clearing and clear images. Images were acquired using a Zeiss LSM 800 confocal microscope.

### Quantification and Statistical Analysis

For all experiments, “n” denotes technical replicates, whereas “N” represents biological replicates. Unless otherwise noted, all analyses were performed in triplicate; N is indicated for each experiment throughout the figure descriptions. Analysis of stress relaxation of the hydrogel was performed using a custom MATLAB script. Analysis of cell aspects (f-actin, pMLCK, integrin β1) were performed using FIJI. Two-tailed t-sets were performed to determine significance. All graphs were drawn using GraphPad Prism 9. Significance levels were set at *p<0.05, **p<0.01, ***p<0.001, and ***p<0.0001. All graphical data were reported.

### References

Adebowale, K. et al. (2021). Enhanced substrate stress relaxation promotes filopodia-mediated cell migration. Nature Materials 20, 1290-1299.

Bayless, K. J., Salazar, R., and Davis, G. E. (2000). RGD-dependent vacuolation and lumen formation observed during endothelial cell morphogenesis in three-dimensional fibrin matrices involves the (v)13(3) and (5) 13(1) integrins. Am. J. Pathol. 156, 1673-1683.

Beamish, J. A., Juliar, B. A., Cleveland, D. S., Busch, M. E., Nimmagadda, L., and Putnam, A. J. (2019). Deciphering the relative roles of matrix metalloprotei-nase- and plasmin-mediated matrix degradation during capillary morphogen-esis using engineered hydrogels. J. Biomed. Mater. Res. B Appl. Biomater. 107, 2507-2516.

Blatchley, M., Park, K. M., and Gerecht, S. (2015). Designer hydrogels for precision control of oxygen tension and mechanical properties. J. Mater. Chem. B Mater. Biol. Med. 3, 7939-7949.

Blatchley, M. R., Hall, F., Wang, S., Pruitt, H. C., and Gerecht, S. (2019). Hypoxia and matrix viscoelasticity sequentially regulate endothelial progenitor cluster-based vasculogenesis. Sci. Adv. 5, eaau7518.

Bloom, R. J., George, J. P., Celedon, A., Sun, S. X., and Wirtz, D. (2008). Mapping local matrix remodeling induced by a migrating tumor cell using three-dimensional multiple-particle tracking. Biophys. J. 95, 4077-4088.

Bordeleau, F., Mason, B. N., Lollis, E. M., Mazzola, M., Zanotelli, M. R., Somasegar, S., Califano, J. P., Montague, C., LaValley, D. J., Huynh, J., et al. (2017). Matrix stiffening promotes a tumor vasculature phenotype. Proc. Natl. Acad. Sci. USA 114, 492-497.

Boussommier-Calleja, A., Atiyas, Y., Haase, K., Headley, M., Lewis, C., and Kamm, R.D. (2019). The effects of monocytes on tumor cell extravasation in a 3D vascularized microfluidic model. Biomaterials 198, 180-193.

Bramwell, V., Anderson, D., Charette, M. & Sarcoma Disease Site (2003). G. Doxorubicin-based chemotherapy for the palliative treatment of adult patients with locally advanced or metastatic soft tissue sarcoma. Cochrane Database of Systematic Reviews.

Brown, M. C., Perrotta, J. A., and Turner, C. E. (1996). Identification of LIM3 as the principal determinant of paxillin focal adhesion localization and characterization of a novel motif on paxillin directing vinculin and focal adhesion kinase binding. J. Cell Biol. 135, 1109-1123.

Brown, T. E., Carberry, B. J., Worrell, B. T., Dudaryeva, O. Y., McBride, M. K., Bowman, C. N., and Anseth, K. S. (2018). Photopolymerized dynamic hydrogels with tunable viscoelastic properties through thioester exchange. Biomaterials 178, 496-503.

Brown, A., He, H., Trumper, E., Valdez, J., Hammond, P., and Griffith, L. G. (2020). Engineering PEG-based hydrogels to foster efficient endothelial network formation in free-swelling and confined microenvironments. Biomaterials 243, 119921.

Carreau, P. J. (1972). Rheological equations from molecular network theories. Trans. Soc. Rheol. 16, 99-127.

Chang, J., and Chaudhuri, O. (2019). Beyond proteases: basement membrane mechanics and cancer invasion. J. Cell Biol. 218, 2456-2469.

Chaudhuri, O., Gu, L., Darnell, M., Klumpers, D., Bencherif, S. A., Weaver, J. C., Huebsch, N., and Mooney, D. J. (2015). Substrate stress relaxation regulates cell spreading. Nat. Commun. 6, 6364.

Chaudhuri, O., Gu, L., Klumpers, D., Darnell, M., Bencherif, S. A., Weaver, J. C., Huebsch, N., Lee, H. P., Lippens, E., Duda, G. N., and Mooney, D. J. (2016). Hydrogels with tunable stress relaxation regulate stem cell fate and activity. Nat. Mater. 15, 326-334.

Chen, Y. C., Lin, R. Z., Qi, H., Yang, Y., Bae, H., Melero-Martin, J. M., and Khademhosseini, A. (2012). Functional human vascular network generated in photocrosslinkable gelatin methacrylate hydrogels. Adv. Funct. Mater. 22, 2027-2039.

Chen, D., Teng, J. M., North, P. E., Lapinski, P. E., and King, P. D. (2019). RASA1-dependent cellular export of collagen IV controls blood and lymphatic vascular development. J. Clin. Invest. 129, 3545-3561.

Chun, T. -H., Sabeh, F., Ota, I., Murphy, H., McDonagh, K. T., Holmbeck, K., Birkedal-Hansen, H., Allen, E. D., and Weiss, S. J. (2004). MT1-MMP-depen-dent neovessel formation within the confines of the three-dimensional extra-cellular matrix. J. Cell Biol. 167, 757-767.

Crosby, C. O., and Zoldan, J. (2019). Mimicking the physical cues of the ECM in angiogenic biomaterials. Regen. Biomater. 6, 61-73.

Damayanti, N. P., Buno, K., Narayanan, N., Voytik Harbin, S. L., Deng, M., and Irudayaraj, J. M. K. (2017). Monitoring focal adhesion kinase phosphorylation dynamics in live cells. Analyst (Lond.) 142, 2713-2716.

Davis, G. E., and Bayless, K. J. (2003). An integrin and Rho GTPase-dependent pinocytic vacuole mechanism controls capillary lumen formation in collagen and fibrin matrices. Microcirculation 10, 27-44.

Davis, G. E., and Camarillo, C. W. (1996). An 2 13 1 integrin-dependent pino-cytic mechanism involving intracellular vacuole formation and coalescence regulates capillary lumen and tube formation in three-dimensional collagen matrix. Exp. Cell Res. 224, 39-51.

Davis, G. E., and Senger, D. R. (2005). Endothelial extracellular matrix: biosynthesis, remodeling, and functions during vascular morphogenesis and neoves-sel stabilization. Circ. Res. 97, 1093-1107.

de Greef, T. F., and Meijer, E. W. (2008). Materials science: supramolecular polymers. Nature 453, 171-173.

Dekkers, J. F. et al. (2019). High-resolution 3D imaging of fixed and cleared organoids. Nature Protocols 14, 1756-1771.

del Rio, A., Perez-Jimenez, R., Liu, R., Roca-Cusachs, P., Fernandez, J. M., and Sheetz, M. P. (2009). Stretching single talin rod molecules activates vincu-lin binding. Science 323, 638-641.

Demetri, G. D. A., Scott; Benjamin, Robert S; Bui, Marilyn M; Casper, Ephraim S. (2010). Soft Tissue Sarcoma: Clinical Practice Guidelines in Oncology. National Comprehensive Cancer Network 8, 630-674.

Discher, D. E., Janmey, P., and Wang, Y. L. (2005). Tissue cells feel and respond to the stiffness of their substrate. Science 310, 1139-1143.

Dupont, S., Morsut, L., Aragona, M., Enzo, E., Giulitti, S., Cordenonsi, M., Zanconato, F., Le Digabel, J., Forcato, M., Bicciato, S., et al. (2011). Role of YAP/TAZ in mechanotransduction. Nature 474, 179-183.

Eisinger-Mathason, T. S., Zhang, M., Qiu, Q., Skuli, N., Nakazawa, M. S., Karakasheva, T., Mucaj, V., Shay, J. E., Stangenberg, L., Sadri, N., et al. (2013). Hypoxia-dependent modification of collagen networks promotes sarcoma metastasis. Cancer Discov. 3, 1190-1205.

Engler, A. J., Griffin, M. A., Sen, S., Bonnemann, C. G., Sweeney, H. L., and Discher, D. E. (2004). Myotubes differentiate optimally on substrates with tissue-like stiffness: pathological implications for soft or stiff microenvironments. J. Cell Biol. 166, 877-887.

Fraley, S. I. et al. (2015). Three-dimensional matrix fiber alignment modulates cell migration and MT1-MMP utility by spatially and temporally directing protrusions. Scientific reports 5, 14580.

Freedman, R. B., and Radda, G. K. (1968). The reaction of 2,4,6-trinitrobenze-nesulphonic acid with amino acids, peptides and proteins. Biochem. J. 108, 383-391.

Fukumura, D. & Jain, R. K. (2007). Tumor microenvironment abnormalities: causes, consequences, and strategies to normalize. Journal of cellular biochemistry 101, 937-949.

Fung, Y. C. (2013). Biomechanics: Mechanical Properties of Living Tissues. (Springer New York).

Geerligs, M., Peters, G. W., Ackermans, P. A., Oomens, C. W., and Baaijens, F. P. (2008). Linear viscoelastic behavior of subcutaneous adipose tissue. Biorheology 45, 677-688.

Ghajar, C. M., Blevins, K. S., Hughes, C. C., George, S. C., and Putnam, A. J. (2006). Mesenchymal stem cells enhance angiogenesis in mechanically viable prevascularized tissues via early matrix metalloproteinase upregulation. Tissue Eng. 12, 2875-2888.

Global Health Estimates 2016: Deaths by Cause, Age, Sex, by Country and by Region, 2000-2016. (World Health Organization, Geneva).

Gong, Z. et al. (2018). Matching material and cellular timescales maximizes cell spreading on viscoelastic substrates. Proceedings of the National Academy of Sciences of the United States of America.

Hanjaya-Putra, D., Yee, J., Ceci, D., Truitt, R., Yee, D., and Gerecht, S. (2010). Vascular endothelial growth factor and substrate mechanics regulate in vitro tubulogenesis of endothelial progenitor cells. J. Cell. Mol. Med. 14, 2436-2447.

Hanjaya-Putra, D., Bose, V., Shen, Y. -I., Yee, J., Khetan, S., Fox-Talbot, K., Steenbergen, C., Burdick, J. A., and Gerecht, S. (2011). Controlled activation of morphogenesis to generate a functional human microvasculature in a synthetic matrix. Blood 118, 804-815.

Hoang, N. T., Acevedo, L. A., Mann, M. J. & Tolani, B. (2018). A review of soft-tissue sarcomas: translation of biological advances into treatment measures. Cancer management and research 10.

Hosseini, A., Rasmi, Y., Rahbarghazi, R., Aramwit, P., Daeihassani, B., and Saboory, E. (2019). Curcumin modulates the angiogenic potential of human endothelial cells via FAK/P-38 MAPK signaling pathway. Gene 688, 7-12.

Hozumi, T., Kageyama, T., Ohta, S., Fukuda, J., and Ito, T. (2018). Injectable hydrogel with slow degradability composed of gelatin and hyaluronic acid cross-linked by Schiff's base formation. Biomacromolecules 19, 288-297.

Huebsch, N., Arany, P. R., Mao, A. S., Shvartsman, D., Ali, O. A., Bencherif, S. A., Rivera-Feliciano, J., and Mooney, D. J. (2010). Hamessing traction-mediated manipulation of the cell/matrix interface to control stem-cell fate. Nat. Mater. 9, 518-526.

Humphries, J. D., Wang, P., Streuli, C., Geiger, B., Humphries, M. J., and Ballestrem, C. (2007). Vinculin controls focal adhesion formation by direct interactions with talin and actin. J. Cell Biol. 179, 1043-1057.

Iruela-Arispe, M. L., and Davis, G. E. (2009). Cellular and molecular mechanisms of vascular lumen formation. Dev. Cell 16, 222-231.

Italiano, A. M. -P., Simone, Cesne, Axel Le; Terrier, Philippe; Bonvalot, Sylvie; Collin, Francoise; Michels, Jean-Jacques; Blay, Jean-Yves; Coindre, Jean-Michel; Bui, Binh. (2010). Trends in survival for patients with metastatic soft-tissue sarcoma. Cancer 117, 1049-1054.

Jemal, A. S., R; Xu J., Ward, E. (2010). Cancer statistics. CA Cancer J Clin 60, 277-300.

Jeon, J. S., Bersini, S., Gilardi, M., Dubini, G., Charest, J. L., Moretti, M., and Kamm, R. D. (2015). Human 3D vascularized organotypic microfluidic assays to study breast cancer cell extravasation. Proc. Natl. Acad. Sci. USA 112, 214-219.

Junttila, M. R. & de Sauvage, F. J. (2013). Influence of tumour micro-environment heterogeneity on therapeutic response. Nature 501, 346-354.

Kang, H. -W., Tabata, Y., and Ikada, Y. (1999). Fabrication of porous gelatin scaffolds for tissue engineering. Biomaterials 20, 1339-1344.

Karavasilis, V. et al. (2008). Significant clinical benefit of first-line palliative chemotherapy in advanced soft-tissue sarcoma. Cancer 112, 1585-1591.

Kim, S. H., Kang, J. G., Kim, C. S., Ihm, S. -H., Choi, M. G., Yoo, H. J., and Lee, S. J. (2014). The hsp70 inhibitor VER155008 induces paraptosis requiring de novo protein synthesis in anaplastic thyroid carcinoma cells. Biochem. Biophys. Res. Commun. 454, 36-41.

Kovács, M., Tóth, J., Hetényi, C., Málnási-Csizmadia, A., and Sellers, J. R. (2004). Mechanism of blebbistatin inhibition of myosin II. J. Biol. Chem. 279, 35557-35563.

Legant, W. R., Miller, J. S., Blakely, B. L., Cohen, D. M., Genin, G. M., and Chen, C. S. (2010). Measurement of mechanical tractions exerted by cells in three-dimensional matrices. Nat. Methods 7, 969-971.

Levental, I., Georges, P. C., and Janmey, P. A. (2007). Soft biological materials and their impact on cell function. Soft Matter 3, 299-306.

Lewis, D. M., Tang, V., Jain, N., Isser, A., Xia, Z., and Gerecht, S. (2018). Collagen fiber architecture regulates hypoxic sarcoma cell migration. ACS Biomater. Sci. Eng. 4, 400-409.

Lewis, D. M., Pruitt, H., Jain, N., Ciccaglione, M., McCaffery, J. M., Xia, Z., Weber, K., Eisinger-Mathason, T. S. K., and Gerecht, S. (2019). A feedback loop between hypoxia and matrix stress relaxation increases oxygen-Axis migration and metastasis in sarcoma. Cancer Res. 79, 1981-1995.

Li, S., Nih, L. R., Bachman, H., Fei, P., Li, Y., Nam, E., Dimatteo, R., Carmichael, S. T., Barker, T. H., and Segura, T. (2017). Hydrogels with precisely controlled integrin activation dictate vascular patterning and permeability. Nat. Mater. 16, 953-961.

Linch, M., Miah, A. B., Thway, K., Judson, I. R. & Benson, C. (2014). Systemic treatment of soft-tissue sarcoma-gold standard and novel therapies. Nature Reviews Clinical Oncology 11, 187.

Liu, Z., and Bilston, L. (2000). On the viscoelastic character of liver tissue: experiments and modelling of the linear behaviour. Biorheology 37, 191-201.

Liu, Z. Q., Wei, Z., Zhu, X. L., Huang, G. Y., Xu, F., Yang, J. H., Osada, Y., Zri'nyi, M., Li, J. H., and Chen, Y. M. (2015). Dextran-based hydrogel formed by thiol-Michael addition reaction for 3D cell encapsulation. Colloids Surf. B Biointerfaces 128, 140-148.

Loebel, C., Mauck, R. L., and Burdick, J. A. (2019). Local nascent protein deposition and remodelling guide mesenchymal stromal cell mechanosensing and fate in three-dimensional hydrogels. Nat. Mater. 18, 883-891.

Lou, J., Stowers, R., Nam, S., Xia, Y., and Chaudhuri, O. (2018). Stress relaxing hyaluronic acid-collagen hydrogels promote cell spreading, fiber remodeling, and focal adhesion formation in 3D cell culture. Biomaterials 154, 213-222.

Madl, C. M., LeSavage, B. L., Dewi, R. E., Dinh, C. B., Stowers, R. S., Khariton, M., Lampe, K. J., Nguyen, D., Chaudhuri, O., Enejder, A., and Heilshorn, S. C. (2017). Maintenance of neural progenitor cell stemness in 3D hydrogels requires matrix remodelling. Nat. Mater. 16, 1233-1242.

Maia, J., Ferreira, L., Carvalho, R., Ramos, M. A., and Gil, M. H. (2005). Synthesis and characterization of new injectable and degradable dextran-based hydrogels. Polymer (Guildf.) 46, 9604-9614.

Marchand, M., Monnot, C., Muller, L., and Germain, S. (2019). Extracellular matrix scaffolding in angiogenesis and capillary homeostasis. Semin. Cell Dev. Biol. 89, 147-156

Matsuoka, S. (1992). Relaxation Phenomena in Polymers (Hanser Publishers).

McCoy, M. G., Seo, B. R., Choi, S., and Fischbach, C. (2016). Collagen I hydro-gel microstructure and composition conjointly regulate vascular network formation. Acta Biomater. 44, 200-208.

McCoy, M. G., Nyanyo, D., Hung, C. K., Goerger, J. P., R Zipfel, W., Williams, R. M., Nishimura, N., and Fischbach, C. (2019). Endothelial cells promote 3D invasion of GBM by IL-8-dependent induction of cancer stem cell properties. Sci. Rep. 9, 9069.

McDonald, S. J., Dooley, P. C., McDonald, A. C., Schuijers, J. A., Ward, A. R., and Grills, B. L. (2009). Early fracture callus displays smooth muscle-like visco-elastic properties ex vivo: implications for fracture healing. J. Orthop. Res. 27, 1508-1513.

Mckinnon, D. D., Domaille, D. W., Cha, J. N., and Anseth, K. S. (2014). Biophysically defined and cytocompatible covalently adaptable networks as viscoelastic 3D cell culture systems. Adv. Mater. 26, 865-872.

Mofrad, M. R., Golji, J., Abdul Rahim, N. A., and Kamm, R. D. (2004). Force-induced unfolding of the focal adhesion targeting domain and the influence of paxillin binding. Mech. Chem. Biosyst. 1, 253-265.

Moon, J. J., Saik, J. E., Poche, R. A., Leslie-Barbick, J. E., Lee, S. -H., Smith, A. A., Dickinson, M. E., and West, J. L. (2010). Biomimetic hydrogels with pro-angiogenic properties. Biomaterials 31, 3840-3847.

Nam, S. et al. (2019). Cell cycle progression in confining microenvironments is regulated by a growth-responsive TRPV4-PI3K/Akt-p27<sup>Kip1</sup> signaling axis. Science Advances 5, eaaw6171.

Nam, S., Stowers, R., Lou, J., Xia, Y., and Chaudhuri, O. (2019). Varying PEG density to control stress relaxation in alginate-PEG hydrogels for 3D cell culture studies. Biomaterials 200, 15-24.

Park, K. M., and Gerecht, S. (2014). Hypoxia-inducible hydrogels. Nat. Commun. 5, 4075.

Pedrosa, A. -R., Bodrug, N., Gomez-Escudero, J., Carter, E. P., Reynolds, L. E., Georgiou, P. N., Fernandez, I., Lees, D. M., Kostourou, V., and Alexopoulou, A. N. (2019). Tumor angiogenesis is differentially regulated by phosphorylation of endothelial cell focal adhesion kinase tyrosines-397 and -861. Cancer Res. https://doi.org/10.1158/0008-5472.CAN-18-3934.

Petit, I., Jin, D., and Rafii, S. (2007). The SDF-1-CXCR4 signaling pathway: a molecular hub modulating neo-angiogenesis. Trends Immunol. 28, 299-307.

Quail, D. F. & Joyce, J. A. (2013). Microenvironmental regulation of tumor progression and metastasis. Nature medicine 19, 1423-1437.

Ren, X. -D., Kiosses, W. B., Sieg, D. J., Otey, C. A., Schlaepfer, D. D., and Schwartz, M. A. (2000). Focal adhesion kinase suppresses Rho activity to promote focal adhesion turnover. J. Cell Sci. 113, 3673-3678.

Ronaldson-Bouchard, K., and Vunjak-Novakovic, G. (2018). Organs-on-a-chip: a fast track for engineered human tissues in drug development. Cell Stem Cell 22, 310-324.

Sacharidou, A., Koh, W., Stratman, A. N., Mayo, A. M., Fisher, K. E., and Davis, G. E. (2010). Endothelial lumen signaling complexes control 3D matrix-specific tubulogenesis through interdependent Cdc42- and MT1-MMP-mediated events. Blood 115, 5259-5269.

Schneider, C. A., Rasband, W. S., and Eliceiri, K. W. (2012). NIH Image to ImageJ: 25 years of image analysis. Nat Methods 9, 671-675.

Shen, Y. -I., Abaci, H. E., Krupsi, Y., Weng, L. -C., Burdick, J. A., and Gerecht, S. (2014). Hyaluronic acid hydrogel stiffness and oxygen tension affect cancer cell fate and endothelial sprouting. Biomater. Sci. 2, 655-665.

Shi, Q., and Boettiger, D. (2003). A novel mode for integrin-mediated signaling: tethering is required for phosphorylation of FAK Y397. Mol. Biol. Cell 14, 4306-4315.

Singer, S., Demetri, G. D., Baldini, E. H. & Fletcher, C. D. M. (2000). Management of soft-tissue sarcomas: an overview and update. The Lancet Oncology 1, 75-85.

Stratman, A. N., Saunders, W. B., Sacharidou, A., Koh, W., Fisher, K. E., Zawieja, D. C., Davis, M. J., and Davis, G. E. (2009). Endothelial cell lumen and vascular guidance tunnel formation requires MT1-MMP-dependent proteolysis in 3-dimensional collagen matrices. Blood 114, 237-247.

Stratman, A. N., Davis, M. J., and Davis, G. E. (2011). VEGF and FGF prime vascular tube morphogenesis and sprouting directed by hematopoietic stem cell cytokines. Blood 117, 3709-3719.

Sun, Z., Guo, S. S., and Fassler, R. (2016). Integrin-mediated mechanotransduction. J Cell Biol. 215, 445-456.

Sun, G., Shen, Y. I., Ho, C. C., Kusuma, S., and Gerecht, S. (2010). Functional groups affect physical and biological properties of dextran-based hydrogels. J. Biomed. Mater. Res. A 93, 1080-1090.

Swift, J., Ivanovska, I. L., Buxboim, A., Harada, T., Dingal, P. C., Pinter, J., Pajerowski, J. D., Spinler, K. R., Shin, J. -W., Tewari, M., et al. (2013). Nuclear lamin-A scales with tissue stiffness and enhances matrix-directed differentiation. Science 341, 1240104.

Tadokoro, S., Shattil, S. J., Eto, K., Tai, V., Liddington, R. C., de Pereda, J. M., Ginsberg, M. H., and Calderwood, D. A. (2003). Talin binding to integrin b tails: a final common step in integrin activation. Science 302, 103-106.

Tang, S., Ma, H., Tu, H. C., Wang, H. R., Lin, P. C., and Anseth, K. S. (2018). Adaptable Fast Relaxing Boronate-Based Hydrogels for Probing Cell-Matrix Interactions. Adv. Sci. (Weinh.) 5, 1800638.

Trappmann, B., Gautrot, J. E., Connelly, J. T., Strange, D. G., Li, Y., Oyen, M. L., Cohen Stuart, M. A., Boehm, H., Li, B., Vogel, V., et al. (2012). Extracellular-matrix tethering regulates stem-cell fate. Nat. Mater. 11, 642-649.

Turturro, M. V., Christenson, M. C., Larson, J. C., Young, D. A., Brey, E. M., and Papavasiliou, G. (2013). MMP-sensitive PEG diacrylate hydrogels with spatial variations in matrix properties stimulate directional vascular sprout formation. PLOS ONE 8, e58897.

Ueki, Y. et al. (2020). PLOD2 Is Essential to Functional Activation of Integrin β1 for Invasion/Metastasis in Head and Neck Squamous Cell Carcinomas. iScience 23, 100850-100850.

Wang, H., and Heilshom, S. C. (2015). Adaptable hydrogel networks with reversible linkages for tissue engineering. Adv. Mater. 27, 3717-3736.

Wang, Y., and McNiven, M. A. (2012). Invasive matrix degradation at focal adhesions occurs via protease recruitment by a FAK-p130Cas complex. J. Cell Biol. 196, 375-385.

Wasif, N. T., Robert M; Christensen, Scott; Do, Ly; Martinez, Steve R; Chen, Steven L; Canter, Robert J. (2011). Influence of Specialty and Clinical Experience on Treatment Sequencing in the Multimodal Management of Soft Tissue Extremity Sarcoma. Annals of Surgical Oncology 19, 504-510.

Wei, Z., and Gerecht, S. (2018). A self-healing hydrogel as an injectable instructive carrier for cellular morphogenesis. Biomaterials 185, 86-96.

Wei, Z., Yang, J. H., Zhou, J., Xu, F., Zri'nyi, M., Dussault, P. H., Osada, Y., and Chen, Y. M. (2014). Self-healing gels based on constitutional dynamic chemistry and their potential applications. Chem. Soc. Rev. 43, 8114-8131.

Wei, Z., Yang, J. H., Liu, Z. Q., Xu, F., Zhou, J. X., Zri'nyi, M., Osada, Y., and Chen, Y. M. (2015). Novel biocompatible polysaccharide based self-healing hydrogel. Adv. Funct. Mater. 25, 1352-1359.

Wei, Z., Lewis, D. M., Xu, Y., and Gerecht, S. (2017). Dual Cross-Linked Biofunctional and Self-Healing Networks to Generate User-Defined Modular Gradient Hydrogel Constructs. Adv. Healthc. Mater. 6, 1700523.

Wei, Z., Schnellmann, R., Pruitt, H. C. & Gerecht, S. (2020). Hydrogel Network Dynamics Regulate Vascular Morphogenesis. Cell Stem Cell 27, 798-812.e796.

Wojtecki, R. J., Meador, M. A., and Rowan, S. J. (2011). Using the dynamic bond to access macroscopically responsive structurally dynamic polymers. Nat. Mater. 10, 14-27.

Yamaguchi, J., Kusano, K. F., Masuo, O., Kawamoto, A., Silver, M., Murasawa, S., Bosch-Marce, M., Masuda, H., Losordo, D. W., Isner, J. M., and Asahara, T. (2003). Stromal cell-derived factor-1 effects on ex vivo expanded endothelial progenitor cell recruitment for ischemic neovascularization. Circulation 107, 1322-1328.

Yoder, M. C., Mead, L. E., Prater, D., Krier, T. R., Mroueh, K. N., Li, F., Krasich, R., Temm, C. J., Prchal, J. T., and Ingram, D. A. (2007). Redefining endothelial progenitor cells via clonal analysis and hematopoietic stem/progenitor cell principals. Blood 109, 1801-1809.

Yoon, C., Choi, C., Stapleton, S., Mirabella, T., Howes, C., Dong, L., King, J., Yang, J., Oberai, A., Eyckmans, J., and Chen, C. S. (2019). Myosin IIA-mediated forces regulate multicellular integrity during vascular sprouting. Mol. Biol. Cell 30, 1974-1984.

Yoon, S. S. et al. (2006). Angiogenic Profile of Soft Tissue Sarcomas Based on Analysis of Circulating Factors and Microarray Gene Expression. Journal of Surgical Research 135, 282-290.

Yu, C. H., Law, J. B., Suryana, M., Low, H. Y., and Sheetz, M. P. (2011). Early integrin binding to Arg-Gly-Asp peptide activates actin polymerization and contractile movement that stimulates outward translocation. Proc. Natl. Acad. Sci. USA 108, 20585-20590.

Zhang, B., Montgomery, M., Chamberlain, M. D., Ogawa, S., Korolj, A., Pahnke, A., Wells, L. A., Masse', S., Kim, J., Reis, L., et al. (2016). Biodegradable scaffold with built-in vasculature for organ-on-a-chip engineering and direct surgical anastomosis. Nat. Mater. 15, 669-678.

Zhao, X., Liu, S., Yildirimer, L., Zhao, H., Ding, R., Wang, H., Cui, W., and Weitz, D. (2016). Injectable stem cell-laden photocrosslinkable microspheres fabricated using microfluidics for rapid generation of osteogenic tissue constructs. Adv. Funct. Mater. 26, 2809-2819.

Zheng, Y., Chen, J., Craven, M., Choi, N. W., Totorica, S., Diaz-Santana, A., Kermani, P., Hempstead, B., Fischbach-Teschi, C., López, J. A., and Stroock, A. D. (2012). In vitro microvessels for the study of angiogenesis and thrombosis. Proc. Natl. Acad. Sci. USA 109, 9342-9347.

Ziegler, W. H., Gingras, A. R., Critchley, D. R., and Emsley, J. (2008). Integrin connections to the cytoskeleton through talin and vinculin. Biochem. Soc. Trans. 36 (Part 2), 235-239.

1. A hydrogel prepared from:
i) a polypeptide with one or more of a primary cross-linkable group; and
ii) a polysaccharide with one or more of a secondary cross-linkable group;
wherein said hydrogel is formed by reacting said primary cross-linkable group with said secondary cross-linkable group.

2. The hydrogel of claim 1, wherein the polypeptide is selected from the group consisting of gelatin, soy protein, albumin, and collagen

3. (canceled)

4. The hydrogel of claim 1, wherein the polysaccharide is selected from the group consisting of hyaluronic acid, agarose, alginate, cellulose and derivatives thereof, methylcellulose, chitosan, and dextran.

5. (canceled)

6. The hydrogel of claim 1, wherein:
i) said primary cross-linkable group is an acylhydrazine; and
ii) said secondary cross-linkable group is an aldehyde.

7. The hydrogel of claim 1, wherein:
said first and second cross-linkable groups are (meth)acrylates;

8. The hydrogel of claim 7, wherein:
said secondary cross-linkable group is a glycidyl (meth)acrylate.

9. The hydrogel of claim 1, wherein:
said polypeptide is gelatin and said primary cross-linkable group is an acylhydrazine; and
said polysaccharide is dextran and said secondary cross-linkable group is an aldehyde.

10. The hydrogel of claim 1, wherein:
said polypeptide is gelatin and said primary cross-linkable group is a methacrylate; and
said polysaccharide is dextran and said secondary cross-linkable group is a glycidyl (meth)acrylate.

11. The hydrogel of claim 1, further comprising a cell or a therapeutic agent.

12. The hydrogel of claim 1, wherein the hydrogel comprises up to 5 wt. % of the primary cross-linkable group and up to 0.5 wt. % of the secondary cross-linkable group.

13. The hydrogel of claim 12, wherein the hydrogel comprises 0.5 wt. % of the secondary cross-linkable group.

14. (canceled)

15. A method of making the hydrogel of claim 9, comprising:
i) combining said gelatin and said dextran in saline by
a) dissolving said gelatin in a saline to form a first solution;
dissolving said dextran in a saline to form a second solution; and mixing together said first solution with said second solution and optionally adding a photoinitiator to form a mixture; or b) mixing together in saline the gelatin, and the dextran, and optionally a photo-initiator to form a mixture; and
ii) crosslinking said mixture to form the hydrogel.

16. The method of claim 10, wherein crosslinking comprises thermally crosslinking, chemically crosslinking, or, when a photoinitiator is present, exposing said mixture light.

17. A method of growing vasculature or other tissue, healing wounds, delivering cells, or delivering a therapeutic agent comprising administering to a subject in need thereof the hydrogel of claim 1.

18. A method comprising
investigating tissue assembly and morphogenesis by measuring a parameter of one or more of vascularization, angiogenesis, cellular migration, stress relaxation, interaction of cellular peptides, and cellular responses to a stimulus in a first hydrogel and a second hydrogel, or
investigating tumor growth, single-cell matrix interactions stress relaxation time changes in tumor-matrix interactions by measuring filopodia-like protrusions in a first hydrogel and a second hydrogel;
wherein the first hydrogel is a dynamic hydrogel and the second hydrogel is a non-dynamic hydrogel; and the first hydrogel and the second hydrogel have similar stiffness;
comparing the parameter or filopodia-like protrusions as measured in the first hydrogel to the parameter as measured in the second hydrogel; and
determining a property of tissue assembly and morphogenesis or tumor growth by the comparison.

19. (canceled)

20. The method of claim 18, wherein the dynamic hydrogel comprises a
i) a polypeptide with a primary cross-linkable group comprising an acylhydrazine; and
ii) a polysaccharide with a secondary cross-linkable group comprising an aldehyde;
wherein said hydrogel is formed by reacting said primary cross-linkable group with said secondary cross-linkable group; and the non-dynamic hydrogel comprises
i) the polypeptide with a primary cross-linkable (meth)acrylate group; and
ii) the polysaccharide with a secondary cross-linkable (meth)acrylate group;
wherein said hydrogel is formed by reacting said primary cross-linkable (meth)acrylate group with said secondary cross-linkable (meth)acrylate group.

21. The method of claim 20, wherein a weight ratio of polypeptide to polysaccharide in the dynamic hydrogel and a weight ratio of polypeptide to polysaccharide in the non-dynamic hydrogel are about the same.

22. The method of claim 20, wherein the weight ratio of polypeptide to polysaccharide in the dynamic hydrogel and the weight ratio of polypeptide to polysaccharide in the non-dynamic hydrogel are each about a 10:1.

23. (canceled)

24. The method of claim 18, wherein the polypeptide is gelatin, and the polysaccharide is dextran.

24. A method of promoting angiogenesis, tissue regeneration, reperfusion or perfusion in a subject or promoting spheroid growth, single-cell matrix interactions, focal adhesions, or filopodia-like protrusions in need thereof comprising administering the hydrogel of claim 1.

25. The method of claim 23, wherein the hydrogel further comprises a cell or a therapeutic agent.

26. (canceled)
